(19) United States
(12) Patent Application Publication
(10) Pub. No.: US 2026/0347953 A1
(43) Pub. Date: Nov. 5, 2026

(54) MODIFIED MOLECULAR BEACONS FOR IMPROVED DETECTION SPECIFICITY

(71) Applicant: Becton, Dickinson and Company, Franklin Lakes, NJ (US)

(72) Inventors: Gregory A. Richart, San Diego, CA (US); Sydney Bryce Tola, San Diego, CA (US)

(21) Appl. No.: 19/106,160

(22) PCT Filed: Sep. 6, 2023
(86) PCT No.: PCT/US2023/073576

§ 371 Date: Feb. 24, 2025

Related U.S. Application Data
(60) Provisional application No. 63/374,772, filed on Sep. 7, 2022.
(60) Provisional application No. 63/374,774, filed on Sep. 7, 2022.

(51) Int. Cl. C12Q 1/70; C12Q 1/689

(52) U.S. Cl. CPC C12Q 1/701; C12Q 1/689

(57) ABSTRACT

Disclosed herein include methods, compositions, and kits for use in detecting a target nucleic acid sequence in a sample. The method can comprise the use of a signal-generating oligonucleotide capable of hybridizing to a nucleic acid amplification product and comprising one or more polymerase stoppers. In some embodiments, assays employing the protected signal-generating oligonucleotides provided herein exhibit reduced non-specific product formation and fewer false positives.

### RELATED APPLICATIONS

The present application a National Stage application of PCT Application PCT/US2023/073576, filed on Sep. 6, 2023, which claims priority to U.S. Provisional Application No. 63/374,772, filed Sep. 7, 2022; and U.S. Provisional Application No. 63/374,774, filed Sep. 7, 2022. The entire contents of these applications are hereby expressly incorporated by reference in their entireties.

### REFERENCE TO SEQUENCE LISTING

The present application is being filed along with a Sequence Listing in electronic format. The Sequence Listing is provided as a file entitled 68EB-317332-WO, created Sep. 6, 2023, which is 115,267 bytes in size. The information in the electronic format of the Sequence Listing is incorporated herein by reference in its entirety.

### BACKGROUND

### Field

The present disclosure relates generally to methods and compositions for amplification (e.g., isothermal amplification) of nucleic acids.

### Description of the Related Art

Nucleic acid-based diagnostics can be useful for rapid detection of infection, disease and/or genetic variations. For example, identification of bacterial or viral nucleic acid in a sample can be useful for diagnosing a particular type of infection. Other examples include identification of single nucleotide polymorphisms for disease management or forensics, and identification of genetic variations indicative of genetically modified food products. Often, nucleic acid-based diagnostic assays require amplification of a specific portion of nucleic acid in a sample. A common technique for nucleic acid amplification is the polymerase chain reaction (PCR). This technique typically requires a cycling of temperatures (i.e., thermocycling) to proceed through the steps of denaturation (e.g., separation of the strands in the double-stranded DNA (dsDNA) complex), annealing of oligonucleotide primers (short strands of complementary DNA sequences), and extension of the primer along a complementary target by a polymerase. Such thermocycling can be a time consuming process that generally requires specialized machinery. Thus, a need exists for quicker nucleic acid amplification methods that can be performed without thermocycling. Additionally, in some embodiments, non-specific product formation can be caused by the unintended interaction of the probe with an amplification primer (followed by extension of said amplification primer), which can lead to false positives. There is a need for compositions and methods of nucleic acid detection wherein unintended extension product formation and false positives are reduced.

### SUMMARY

Disclosed herein include methods for detecting a target nucleic acid sequence in a sample. In some embodiments, the method comprises: amplifying a target nucleic acid sequence in an amplification reaction mixture under an isothermal amplification condition, thereby generating a nucleic acid amplification product; and detecting the nucleic acid amplification product with a signal-generating oligonucleotide, wherein the signal-generating oligonucleotide is capable of hybridizing to the nucleic acid amplification product and comprises one or more polymerase stoppers. The method can comprise: contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence. The method can comprise: contacting a reagent composition with the treated sample to generate the amplification reaction mixture, wherein the reagent composition comprises one or more amplification reagents.

Disclosed herein include signal-generating oligonucleotides. In some embodiments, the signal-generating oligonucleotide is capable of hybridizing to a nucleic acid amplification product. In some embodiments, the signal-generating oligonucleotide comprises a 5′ subdomain and a 3′ subdomain. In some embodiments, the signal-generating oligonucleotide comprises a loop domain situated between the 5′ subdomain and the 3′ subdomain. In some embodiments, intramolecular nucleotide base pairing between the 5′ subdomain and the 3′ subdomain are capable of forming a paired stem domain. In some embodiments, the loop domain comprises one or more polymerase stoppers. In some embodiments, the 5′ subdomain, the paired stem domain, and/or the 3′ subdomain does not comprise the one or more polymerase stoppers. In some embodiments, the nucleic acid amplification product is generated by amplifying a target nucleic acid sequence comprising a first strand and a second strand complementary to each other. In some embodiments, the nucleic acid amplification product is generated by amplifying the target nucleic acid sequence with the forward primer and the reverse primer.

The signal-generating oligonucleotide can comprise a 5′ subdomain and a 3′ subdomain. In some embodiments, the signal-generating oligonucleotide comprises a loop domain situated between the 5′ subdomain and the 3′ subdomain. In some embodiments, intramolecular nucleotide base pairing between the 5′ subdomain and the 3′ subdomain are capable of forming a paired stem domain. In some embodiments, the one or more polymerase stoppers are situated in the loop domain. In some embodiments, the 5′ subdomain, the paired stem domain, and/or the 3′ subdomain does not comprise the one or more polymerase stoppers. In some embodiments, the nucleic acid amplification product comprises: (1) the sequence of a forward primer, and the reverse complement thereof, (2) the sequence of a reverse primer, and the reverse complement thereof, and (3) a spacer sequence flanked by (1) the sequence of the forward primer and the reverse complement thereof and (2) the sequence of the reverse primer and the reverse complement thereof. In some embodiments, the spacer sequence is 1 to 10 bases long. In some embodiments, the signal-generating oligonucleotide comprises a first region comprising the sequence of at least a portion of the reverse primer. In some embodiments, the signal-generating oligonucleotide comprises a second region comprising a sequence complementary to at least a portion of the forward primer. In some embodiments, the signal-generating oligonucleotide comprises a spacer region comprising the sequence of at least a portion of the spacer sequence. One or more of the first region, the second region, and/or the spacer region can comprise one or more polymerase stoppers. The first region can comprise a sequence complementary to at least two 3′ terminal nucleotides of the forward primer and/or the reverse primer. The second region can comprise a sequence complementary to at least two 3′ terminal nucleotides of the forward primer and/or the reverse primer. In some embodiments, the spacer region comprises a sequence complementary to at least two 3′ terminal nucleotides of the forward primer and/or the reverse primer.

The signal-generating oligonucleotide can be about 10 nucleotides to about 100 nucleotides in length. In some embodiments, the forward primer and/or the reverse primer is about 5 nucleotides to about 25 nucleotides in length. In some embodiments, the second region, the spacer region, and/or the first region is about 1 nucleotide to about 25 nucleotides in length. In some embodiments, the 5′ subdomain, the 3′ subdomain, the loop domain, the 5′ terminal domain, and/or the 3′ terminal domain is about 1 nucleotide to about 25 nucleotides in length. In some embodiments, the signal-generating oligonucleotide comprises a 5′ terminal domain situated 5′ of the 5′ subdomain. In some embodiments, the signal-generating oligonucleotide comprises a 3′ terminal domain situated 3′ of the 5′ subdomain. In some embodiments, the 5′ terminal domain and/or the 3′ terminal domain does not comprise the one or more polymerase stoppers.

In some embodiments, the first region comprises at least a portion of the 5′ subdomain and/or loop domain. In some embodiments, the spacer region comprises at least a portion of the loop domain. In some embodiments, the second region comprises at least a portion of the loop domain and/or 3′ subdomain. In some embodiments, the 5′ subdomain comprises at least a portion of the first region and/or spacer region. In some embodiments, the loop domain comprises at least a portion of the spacer region, the first region, and/or the second region. In some embodiments, the 3′ subdomain comprises at least a portion of the second region and/or spacer region.

The one or more polymerase stoppers can comprise one or more 2′-O-methyl (2′OM) RNA nucleotides. In some embodiments, the one or more polymerase stoppers comprise one or more of an abasic site, a stable abasic site, a chemically trapped abasic site, or any combination thereof. In some embodiments, the stable abasic site comprises 1′,2′-dideoxy. In some embodiments, the chemically trapped abasic site comprises an abasic site reacted with alkoxy amine or sodium borohydride. In some embodiments, the abasic site comprises an apurinic site, an apyrimidinic site, or both. In some embodiments, the abasic site is generated by an alkylating agent or an oxidizing agent. In some embodiments, the one or polymerase stoppers comprise: one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methyl cytidines, one or more 5-hydroxymethylcytidines, one or more 2′-O-Methyl RNA bases, one or more unmethylated RNA bases, one or more Iso-deoxycytidines (Iso-dCs), one or more Iso-deoxyguanosines (Iso-dGs), one or more C3(OC₃H₆OPO₃) groups, one or more photo-cleavable (PC) [OC₃H₆—C(o)NHCH₂—C₆H₃NO₂—CH(CH₃)OPO₃] groups, one or more hexandiol groups, one or more spacer 9 (iSp9) [(OCH₂CH₂)₃OPO₃] groups, one or more spacer 18 (iSp18) [(OCH₂CH₂₆OPO₃] groups, or any combination thereof.

In some embodiments, the signal-generating oligonucleotide comprises one or more phosphorothioate linkages and/or one or more locked nucleic acids. In some embodiments, the signal-generating oligonucleotide is a TaqMan detection probe oligonucleotide, a molecular beacon detection probe oligonucleotide, or a molecular torch detection probe oligonucleotide. In some embodiments, the signal-generating oligonucleotide comprises a label, for example a quenchable label (e.g., a fluorophore). In some embodiments, the signal-generating oligonucleotide comprises a quencher. In some embodiments, the label is situated in the 3′ terminal domain and the quencher is situated in the 5′ terminal domain, and/or the label is situated in the 5′ terminal domain and the quencher is situated in the 3′ terminal domain.

In some embodiments, upon the forward primer binding the signal-generating oligonucleotide to form a first undesirable duplex, the one or more polymerase stoppers are capable of stopping polymerase extension of the forward primer of the first undesirable duplex to the 5′ end of the signal-generating oligonucleotide. In some embodiments, the one or more polymerase stoppers are capable of stopping polymerase extension of the forward primer of the first undesirable duplex beyond the one or more polymerase stoppers of the signal-generating oligonucleotide. In some embodiments, upon the reverse primer binding the signal-generating oligonucleotide to form a second undesirable duplex, the one or more polymerase stoppers are capable of stopping polymerase extension of the reverse primer of the second undesirable duplex to the 5′ end of the signal-generating oligonucleotide. In some embodiments, the one or more polymerase stoppers are capable of stopping polymerase extension of the reverse primer of the second undesirable duplex beyond the one or more polymerase stoppers of the signal-generating oligonucleotide. In some embodiments, upon an extraneous nucleic acid binding the signal-generating oligonucleotide to form a third undesirable duplex, the one or more polymerase stoppers are capable of stopping polymerase extension of the extraneous nucleic acid of the third undesirable duplex to the 5′ end of the signal-generating oligonucleotide. In some embodiments, the one or more polymerase stoppers are capable of stopping polymerase extension of the extraneous nucleic acid of the third undesirable duplex beyond the one or more polymerase stoppers of the signal-generating oligonucleotide. In some embodiments, the extraneous nucleic acid is selected from the group comprising a sample nucleic acid, a primer configured to hybridize a second target nucleic acid sequence, a primer configured to hybridize an internal control, or any combination thereof.

In some embodiments, the sample nucleic acids comprise a nucleic acid comprising the target nucleic acid sequence. In some embodiments, amplifying the target nucleic acid sequence comprises: amplifying a target nucleic acid sequence comprising a first strand and a second strand complementary to each other in an isothermal amplification condition, wherein the amplifying comprises contacting a nucleic acid comprising the target nucleic acid sequence with: i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and ii) an enzyme having a hyperthermophile polymerase activity, thereby generating the nucleic acid amplification product. In some embodiments, the nucleic acid is a double-stranded DNA. In some embodiments, the nucleic acid is a product of reverse transcription reaction. In some embodiments, the nucleic acid is a product of reverse transcription reaction generated from sample ribonucleic acids. In some embodiments, the amplifying comprises generating the nucleic acid by a reverse transcription reaction. In some embodiments, the sample nucleic acids comprise sample ribonucleic acids, and wherein the method comprises contacting sample ribonucleic acids with a reverse transcriptase and/or a reverse transcription primer to generate a cDNA. In some embodiments, amplifying the target nucleic acid sequence comprises: (c1) contacting sample ribonucleic acids with a reverse transcriptase and/or a reverse transcription primer to generate a cDNA; (c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other; (c3) amplifying the target nucleic acid sequence under an isothermal amplification condition, wherein the amplifying comprises contacting the dsDNA with: (i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and (ii) the enzyme having a hyperthermophile polymerase activity, thereby generating the nucleic acid amplification product.

In some embodiments, if the forward primer binds the signal-generating oligonucleotide to form a first undesirable duplex, extension of the forward primer of the first undesirable duplex to the 5′ end of the signal-generating oligonucleotide by an enzyme having a hyperthermophile polymerase activity generates a first undesirable extension product. In some embodiments, the first undesirable extension product is capable of being amplified by the enzyme having a hyperthermophile polymerase activity in the presence of the forward primer and the reverse primer to form a first undesirable amplification product. In some embodiments, the one or more polymerase stoppers are capable of stopping polymerase extension of the forward primer of the first undesirable duplex to generate a first stalled extension product. In some embodiments, the first stalled extension product is not capable of being amplified by the enzyme having a hyperthermophile polymerase activity in the presence of the forward primer and reverse primer to generate the first undesirable amplification product. In some embodiments, the one or more polymerase stoppers are capable of stopping polymerase extension of the forward primer of the first undesirable duplex beyond the one or more polymerase stoppers of the signal-generating oligonucleotide. In some embodiments, if the reverse primer binds the signal-generating oligonucleotide to form a second undesirable duplex, extension of the reverse primer of the second undesirable duplex to the 5′ end of the signal-generating oligonucleotide by an enzyme having a hyperthermophile polymerase activity generates a second undesirable extension product. In some embodiments, the second undesirable extension product is capable of being amplified by the enzyme having a hyperthermophile polymerase activity in the presence of the reverse primer to form a second undesirable amplification product. In some embodiments, the one or more polymerase stoppers are capable of stopping polymerase extension of the reverse primer of the second undesirable duplex to generate a second stalled extension product. In some embodiments, the second stalled extension product is not capable of being amplified by the enzyme having a hyperthermophile polymerase activity in the presence of the reverse primer to generate the second undesirable amplification product. In some embodiments, the one or more polymerase stoppers are capable of stopping polymerase extension of the reverse primer of the second undesirable duplex beyond the one or more polymerase stoppers of the signal-generating oligonucleotide. In some embodiments, if an extraneous nucleic acid binds the signal-generating oligonucleotide to form a third undesirable duplex, extension of the extraneous nucleic acid of the third undesirable duplex to the 5′ end of the signal-generating oligonucleotide by an enzyme having a hyperthermophile polymerase activity generates a third undesirable extension product. In some embodiments, the third undesirable extension product is capable of being amplified by the enzyme having a hyperthermophile polymerase activity in the presence of the reverse primer to form a third undesirable amplification product. In some embodiments, the one or more polymerase stoppers are capable of stopping polymerase extension of the extraneous nucleic acid of the third undesirable duplex to generate a third stalled extension product. In some embodiments, the third stalled extension product is not capable of being amplified by the enzyme having a hyperthermophile polymerase activity in the presence of the reverse primer to generate the third undesirable amplification product. In some embodiments, the one or more polymerase stoppers are capable of stopping polymerase extension of the extraneous nucleic acid of the third undesirable duplex beyond the one or more polymerase stoppers of the signal-generating oligonucleotide.

In some embodiments, the detecting step comprises contacting the nucleic acid amplification product with the signal-generating oligonucleotide for hybridization. In some embodiments, detecting the nucleic acid amplification product comprises use of a real-time detection method. In some embodiments, the label is capable of generating a signal upon the signal-generating oligonucleotide hybridizing the nucleic acid amplification product. In some embodiments, upon the signal-generating oligonucleotide hybridizing the nucleic acid amplification product, the label generates a signal (e.g., fluorescence). In some embodiments, the detecting step comprises detecting the signal of the label before the amplification reaction, after the amplification reaction, or both. In some embodiments, detecting the nucleic acid amplification product comprises detecting a signal generated by the label of the signal-generating oligonucleotide. In some embodiments, the label is a fluorophore and the signal is fluorescence. In some embodiments, detecting a signal comprises detecting fluorescence emitted by the label. In some embodiments, the method comprises determining the presence, absence and/or amount of the target nucleic acid sequence in the sample. In some embodiments, determining the presence, absence and/or amount of the target nucleic acid sequence in the sample comprises determining the presence, absence and/or amount of the dsDNA and/or nucleic acid that comprises the target nucleic acid sequence in the sample. The presence, absence and/or amount of the signal can indicate the presence, absence and/or amount of the target nucleic acid sequence in the sample. In some embodiments, the presence, absence and/or amount of the signal indicates the presence, absence and/or amount of the dsDNA and/or nucleic acid that comprises the target nucleic acid sequence in the sample.

In some embodiments, the label is capable of generating a false positive signal upon the signal-generating oligonucleotide hybridizing the first undesirable amplification product, the second undesirable amplification product, and/or the third undesirable amplification product. In some embodiments, upon the signal-generating oligonucleotide hybridizing the first undesirable amplification product, the second undesirable amplification product, and/or the third undesirable amplification product, the label generates a false positive signal. In some embodiments, the signal and the false positive signal are indistinguishable. In some embodiments, the generation of the first undesirable amplification product, the second undesirable amplification product, and/or the third undesirable amplification product reduces the likelihood of an accurate determination of the presence, absence and/or amount of the target nucleic acid sequence in the sample. In some embodiments, the detection of the false positive signal reduces the likelihood of an accurate determination of the presence, absence and/or amount of the target nucleic acid sequence in the sample.

The presence of the one or more polymerase stoppers in the signal-generating oligonucleotide can increase the likelihood of an accurate determination of the presence, absence and/or amount of the target nucleic acid sequence in the sample by at least about 1.1-fold as compared to a signal-generating oligonucleotide which does not comprise the one or more polymerase stoppers. In some embodiments, the generation of the first stalled extension product, the second stalled extension product, and/or third stalled extension product does not yield a false positive signal. In some embodiments, the signal-generating oligonucleotide hybridizing the first stalled extension product, the second stalled extension product, and/or the third stalled extension product does not generate a false positive signal. In some embodiments, the nucleic acid amplification product reaches detectable levels at, or at least about, 1, 2, 5, 10, 15, or 20 minutes, before the first undesirable amplification product, the second undesirable amplification product, and/or the third undesirable amplification product reaches detectable levels. In some embodiments, the signal reaches detectable levels at, or at least about, 1, 2, 5, 10, 15, or 20 minutes, before the false positive signal reaches detectable levels. In some embodiments, the appearance of detectable levels of the false positive signal, the first undesirable amplification product, the second undesirable amplification product, and/or the third undesirable amplification product is delayed by, or by at least about, 1, 2, 5, 10, 15, or 20 minutes, as compared to a comparable method wherein the signal-generating oligonucleotide which does not comprise the one or more polymerase stoppers. In some embodiments, the false positive signal, the first undesirable amplification product, the second undesirable amplification product, and/or the third undesirable amplification product does not reach detectable levels for, or for at least about, 5, 10, 15, or 20 minutes, after the amplifying step begins. In some embodiments, the generation of the false positive signal, the first undesirable amplification product, the second undesirable amplification product, and/or the third undesirable amplification product is reduced by at least about 1.1-fold as compared to a comparable method wherein the signal-generating oligonucleotide which does not comprise the one or more polymerase stoppers.

In some embodiments, amplifying the target nucleic acid sequence comprises generating the nucleic acid amplification product at detectable levels within, or within about, 20, 15, or 10 minutes. In some embodiments, the detecting is performed in less than about 20 minutes, less than about 15 minutes, less than about 10 minutes, or less than about 5 minutes, from the time the reagent composition is contacted with the treated sample.

In some embodiments, the lysis buffer comprises one or more of magnesium sulfate, ammonium sulfate, EDTA, and EGTA. In some embodiments, the pH of the lysis buffer is about 1.0 to about 10.0 (e.g., about 2.2). In some embodiments, the sample nucleic acids comprise sample ribonucleic acids and/or sample deoxyribonucleic acids. In some embodiments, the sample nucleic acids comprise cellular RNA, mRNA, microRNA, bacterial RNA, viral RNA, or a combination thereof. In some embodiments, the one or more amplification reagents comprise: a reverse transcriptase; an enzyme having a hyperthermophile polymerase activity; and/or dNTPS. In some embodiments, the enzyme having a hyperthermophile polymerase activity has a reverse transcriptase activity a forward primer; a reverse primer; a reverse transcription primer.

The reagent composition can be lyophilized, heat-dried, and/or comprises one or more additives. In some embodiments, the one or more additives comprise: Tween 20, Triton X-100, and/or tween 80; an amino acid; a sugar or sugar alcohol; and/or a polymer. The sugar or sugar alcohol can comprise sucrose, lactose, trehalose, dextran, erythritol, arabitol, xylitol, sorbitol, mannitol, or any combination thereof. In some embodiments, the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof. In some embodiments, contacting the reagent composition with the treated sample comprises dissolving the reagent composition in the treated sample. In some embodiments, the one or more lytic reagents comprise: about 0.001% (w/v) to about 1.0 (w/v) of the treated sample (e.g., about 0.2% (w/v) of the treated sample); and/or a detergent (e.g., one or more of a cationic surfactant, an anionic surfactant, a non-ionic surfactant, and an amphoteric surfactant). In some embodiments, the method: is performed in a single reaction vessel; does not comprise using any enzymes other than the reverse transcriptase and the enzyme having a hyperthermophile polymerase activity; does not comprise using any enzyme other than the enzyme having a hyperthermophile polymerase activity; does not comprise heat denaturing and/or enzymatic denaturing the nucleic acid during the amplification step; and/or does not comprise contacting the nucleic acid with a single-stranded DNA binding protein.

The target nucleic acid sequence can comprise a length of no longer than about 20 nucleotides to no longer than about 90 nucleotides (e.g., about 30 nucleotides). In some embodiments, the forward primer, the reverse primer, and/or the reverse transcription primer is about 8 to 16 bases long. In some embodiments, the nucleic acid amplification product is about 20 to 40 bases long. In some embodiments, the spacer sequence comprises a portion of the target nucleic acid sequence. In some embodiments, the spacer sequence is 1 to 10 bases long. In some embodiments, the isothermal amplification condition comprises a constant temperature of about 30 °C to about 72 °C, for example about 55 °C to about 75 °C, or about 56 °C to about 67 °C. In some embodiments, the amplifying is performed: for a period of about 5 minutes to about 60 minutes (e.g., a period of about 15 minutes). In some embodiments, the amplifying is performed: in helicase-free, single-stranded binding protein-free, cleavage agent-free, and recombinase-free, isothermal amplification conditions. In some embodiments, the amplifying is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA). In some embodiments, the PCR is real-time PCR and/or quantitative real-time PCR (QRT-PCR).

In some embodiments, the enzyme having a hyperthermophile polymerase activity has an amino acid sequence that is at least about 90% or at least about 95% identical to the amino acid sequence of SEQ ID NO: 7 or a functional fragment thereof. In some embodiments, the enzyme having a hyperthermophile polymerase activity is a polymerase comprising the amino acid sequence of SEQ ID NO: 7. In some embodiments, the enzyme having a hyperthermophile polymerase activity has low or no exonuclease activity. In some embodiments, the sample ribonucleic acids are contacted with the reverse transcriptase and the enzyme having a hyperthermophile polymerase activity simultaneously. In some embodiments, the sample ribonucleic acids are contacted with the reverse transcriptase, the enzyme having a hyperthermophile polymerase activity, and the forward and reverse primers simultaneously. In some embodiments, the sample ribonucleic acids are contacted with the reverse transcriptase, the enzyme having a hyperthermophile polymerase activity, the forward primer, the reverse primer, and the reverse transcription primer simultaneously.

The biological entities can comprise one or more of prokaryotic cells, eukaryotic cells, viral particles, exosomes, protoplasts, and microvesicles. In some embodiments, the biological entities comprise a virus, a bacteria, a fungi, a protozoa, portions thereof, or any combination thereof. In some embodiments, the target nucleic acid sequence is a nucleic acid sequence of a virus, bacteria, fungi, or protozoa. In some embodiments, the sample nucleic acids are derived from a virus, bacteria, fungi, or protozoa. The virus can be SARS-CoV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), or Hantavirus. In some embodiments, the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes. In some embodiments, the fungi comprises one or more of Cryptococcus neoformans, Pneumocystis carinii, Histoplasma capsulatum, Blastomyces dermatitidis, Coccidioides immitis, and Trichophyton rubrum. In some embodiments, the protozoa comprises one or more of Trypanosoma cruzi, Leishmania sp., Plasmodium, Entamoeba histolytica, Babesia microti, Giardia lamblia, Cyclospora sp., and Eimeria sp. The sample can be a biological sample or an environmental sample. In some embodiments, the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. In some embodiments, the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

The amplifying step can comprise multiplex amplification of two or more target nucleic acid sequences, and wherein the detecting step comprises multiplex detection of two or more nucleic acid amplification products derived from said two or more target nucleic acid sequences, optionally the two or more target nucleic acid sequences are specific to two or more different organisms, further optionally the two or more different organisms comprise one or more of SARS-CoV-2, Influenza A, Influenza B, and/or Influenza C. In some embodiments, the amplifying comprises and/or does not comprise one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA). In some embodiments, the amplifying does not comprise LAMP. In some embodiments, the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

Disclosed herein include kits for detecting a target nucleic acid sequence in a sample. In some embodiments, the kit comprises: a signal-generating oligonucleotide disclosed herein. The kit can comprise: a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent, and wherein the detergent comprises one or more of a cationic surfactant, an anionic surfactant, a non-ionic surfactant, and an amphoteric surfactant. The kit can comprise: a reagent composition comprising one or more amplification reagents comprising one or more components for amplifying the target nucleic acid sequence under isothermal amplification conditions, wherein said one or more components for amplifying comprise: (i) a forward primer and a reverse primer, wherein the forward primer is capable of hybridizing to a sequence of a first strand of the target nucleic acid sequence, and the reverse primer is capable of hybridizing to a sequence of a second strand of the target nucleic acid sequence; and/or (ii) an enzyme having a hyperthermophile polymerase activity capable of generating a nucleic acid amplification product, optionally the enzyme having a hyperthermophile polymerase activity has an amino acid sequence that is at least about 90% identical to the amino acid sequence of SEQ ID NO: 7 or a functional fragment thereof, optionally the enzyme having a hyperthermophile polymerase activity has an amino acid sequence that is at least about 95% identical to the amino acid sequence of SEQ ID NO: 7, further optionally the enzyme having a hyperthermophile polymerase activity is a polymerase comprising the amino acid sequence of SEQ ID NO: 7. In some embodiments, the reagent composition comprises a reverse transcriptase and/or a reverse transcription primer.

Disclosed herein include methods for detecting N. Gonorrhea in a sample. In some embodiments, the method comprises: contacting said sample with at least one pair of primers, wherein said at least one pair of primers is capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity to any one of the sequences of SEQ ID NOs: 2-3 and 16-17. The method can comprise: generating amplicons (e.g., nucleic acid amplification products) of the target nucleic acid sequence, if said sample comprises N. Gonorrhea. The method can comprise: determining the presence or amount of said amplicons as an indication of the presence of N. Gonorrhea in said sample.

In some embodiments, the at least one pair of primers comprises a first primer comprising the sequence of SEQ ID NOs: 2 or 17, and a second primer comprising the sequence of SEQ ID NOs: 3 or 16. In some embodiments, the at least one pair of primers capable of hybridizing to the target nucleic acid sequence of N. Gonorrhea is SEQ ID NOs: 2 and 3, SEQ ID NOs: 2 and 16, SEQ ID NOs: 17 and 3, or SEQ ID NOs: 17 and 16. In some embodiments, determining the presence or amount of amplicons of the target nucleic acid sequence comprises contacting the amplicons with one or more the signal-generating oligonucleotides, wherein each of the one or more the signal-generating oligonucleotides comprises a sequence selected from the group consisting of SEQ ID NOs: 1 and 12-14, or a sequence that exhibits at least about 85% identity to a sequence selected from the group consisting of SEQ ID NOs: 1 and 12-14. In some embodiments, each of the one or more the signal-generating oligonucleotides comprises a sequence selected from the group consisting of SEQ ID NOs: 1 and 12-14. In some embodiments, each of the one or more the signal-generating oligonucleotides consists of a sequence selected from the group consisting of SEQ ID NOs: 1 and 12-14.

Disclosed herein include methods for detecting C. trachomatis in a sample. In some embodiments, the method comprises: contacting said sample with at least one pair of primers, wherein said at least one pair of primers is capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity to any one of the sequences of SEQ ID NOs: 20-23, 25-26, 28-29, and 31. The method can comprise: generating amplicons (e.g., nucleic acid amplification products) of the target nucleic acid sequence, if said sample comprises C. trachomatis. The method can comprise: determining the presence or amount of said amplicons as an indication of the presence of C. trachomatis in said sample.

In some embodiments, the at least one pair of primers comprises a first primer comprising the sequence of SEQ ID NOs: 20, 22, 25, or 28, and a second primer comprising the sequence of SEQ ID NOs: 21, 23, 26, 29, or 31. In some embodiments, the at least one pair of primers capable of hybridizing to the target nucleic acid sequence of C. trachomatis is SEQ ID NOs: 20 and 21, SEQ ID NOs: 20 and 23, SEQ ID NOs: 20 and 26, SEQ ID NOs: 20 and 29, SEQ ID NOs: 20 and 31, SEQ ID NOs: 22 and 21, SEQ ID NOs: 22 and 23, SEQ ID NOs: 22 and 26, SEQ ID NOs: 22 and 29, SEQ ID NOs: 22 and 31, SEQ ID NOs: 25 and 21, SEQ ID NOs: 25 and 23, SEQ ID NOs: 25 and 26, SEQ ID NOs: 25 and 29, SEQ ID NOs: 25 and 31, SEQ ID NOs: 28 and 21, SEQ ID NOs: 28 and 23, SEQ ID NOs: 28 and 26, SEQ ID NOs: 28 and 29, or SEQ ID NOs: 28 and 31. In some embodiments, determining the presence or amount of amplicons of the target nucleic acid sequence comprises contacting the amplicons with one or more the signal-generating oligonucleotides, wherein each of the one or more the signal-generating oligonucleotides comprises a sequence selected from the group consisting of SEQ ID NOs: 15, 18-19, 24, 27, 30, and 32, or a sequence that exhibits at least about 85% identity to a sequence selected from the group consisting of SEQ ID NOs: 15, 18-19, 24, 27, 30, and 32. In some embodiments, each of the one or more the signal-generating oligonucleotides comprises a sequence selected from the group consisting of SEQ ID NOs: 15, 18-19, 24, 27, 30, and 32. In some embodiments, each of the one or more the signal-generating oligonucleotides consists of a sequence selected from the group consisting of SEQ ID NOs: 15, 18-19, 24, 27, 30, and 32.

Disclosed herein include methods for detecting an Influenza B virus in a sample. In some embodiments, the method comprises: contacting said sample with at least one pair of primers, wherein said at least one pair of primers is capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity to any one of the sequences of SEQ ID NOs: 36-37 and 41. The method can comprise: generating amplicons (e.g., nucleic acid amplification products) of the target nucleic acid sequence, if said sample comprises an Influenza B virus. The method can comprise: determining the presence or amount of said amplicons as an indication of the presence of an Influenza B virus in said sample.

In some embodiments, the at least one pair of primers comprises a first primer comprising the sequence of SEQ ID NO: 36, and a second primer comprising the sequence of SEQ ID NOs: 37 or 41. In some embodiments, the at least one pair of primers capable of hybridizing to the target nucleic acid sequence of an Influenza B virus is SEQ ID NOs: 36 and 37, or SEQ ID NOs: 36 and 41. In some embodiments, determining the presence or amount of amplicons of the target nucleic acid sequence comprises contacting the amplicons with one or more the signal-generating oligonucleotides, wherein each of the one or more the signal-generating oligonucleotides comprises a sequence selected from the group consisting of SEQ ID NOs: 39-40 and 42-45, or a sequence that exhibits at least about 85% identity to a sequence selected from the group consisting of SEQ ID NOs: 39-40 and 42-45. In some embodiments, each of the one or more the signal-generating oligonucleotides comprises a sequence selected from the group consisting of SEQ ID NOs: 39-40 and 42-45. In some embodiments, each of the one or more the signal-generating oligonucleotides consists of a sequence selected from the group consisting of SEQ ID NOs: 39-40 and 42-45. The method can comprise: contacting sample ribonucleic acids of the sample with a reverse transcriptase and a primer exhibiting at least about 85% identity to SEQ ID NO: 38.

In some embodiments, the sample is a biological sample or an environmental sample. In some embodiments, the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. In some embodiments, the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof. In some embodiments, the sample is contacted with a reagent composition comprising the at least one pair of primers to generate the amplification reaction mixture. The method can comprise: contacting the sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence; and contacting a reagent composition comprising the at least one pair of primers with the treated sample to generate the amplification reaction mixture, wherein the reagent composition comprises one or more amplification reagents. In some embodiments, the one or more amplification reagents comprise: a reverse transcriptase; an enzyme having a hyperthermophile polymerase activity, optionally the enzyme having a hyperthermophile polymerase activity has a reverse transcriptase activity; a reverse transcription primer; and/or dNTPs.

In some embodiments, generating amplicons of the target nucleic acid sequence comprises: amplifying the target nucleic acid sequence in the amplification reaction mixture under an amplification condition, thereby generating amplicons of the target nucleic acid sequence. In some embodiments, the amplifying is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA), optionally the PCR is real-time PCR and/or quantitative real-time PCR (QRT-PCR). In some embodiments, the amplifying does not comprise one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA), optionally the amplifying does not comprise LAMP. In some embodiments, the amplifying comprises one or more of the following: APA, LAMP, HDA, RPA, SDA, NASBA, TMA, NEAR, RCA, MDA, RAM, cHDA, SPIA, SMART, 3SR, GEAR and IMDA, optionally the amplifying does not comprise LAMP.

In some embodiments, the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

The determining step can comprise contacting the amplicons of the target nucleic acid sequence with the signal-generating oligonucleotide for hybridization, optionally the determining comprises use of a real-time detection method. In some embodiments, a label of the signal-generating oligonucleotide is capable of generating a signal upon the signal-generating oligonucleotide hybridizing the amplicons of the target nucleic acid sequence. In some embodiments, upon the signal-generating oligonucleotide hybridizing the amplicons of the target nucleic acid sequence, the label generates a signal, optionally the signal is fluorescence.

Disclosed herein include compositions for the detection of N. Gonorrhea in a sample. In some embodiments, the composition comprises: at least one pair of primers capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity to any one of the sequences of SEQ ID NOs: 2-3 and 16-17. In some embodiments, the at least one pair of primers capable of hybridizing to the target nucleic acid sequence of N. Gonorrhea comprises a primer comprising the sequence of SEQ ID NOs: 2 or 17 and a primer comprising the sequence of SEQ ID NOs: 3 or 16. The composition can comprise: one or more the signal-generating oligonucleotides, wherein each of the one or more the signal-generating oligonucleotides comprises a sequence selected from the group consisting of SEQ ID NOs: 1 and 12-14, or a sequence that exhibits at least about 85% identity to a sequence selected from the group consisting of SEQ ID NOs: 1 and 12-14. In some embodiments, each of the one or more the signal-generating oligonucleotides comprises a sequence selected from the group consisting of SEQ ID NOs: 1 and 12-14. In some embodiments, each of the one or more the signal-generating oligonucleotides consists of a sequence selected from the group consisting of SEQ ID NOs: 1 and 12-14.

Disclosed herein include compositions for the detection of C. trachomatis in a sample. In some embodiments, the composition comprises: at least one pair of primers capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity to any one of the sequences of SEQ ID NOs: 20-23, 25-26, 28-29, and 31. In some embodiments, the at least one pair of primers capable of hybridizing to the target nucleic acid sequence of C. trachomatis comprises a primer comprising the sequence of SEQ ID NOs: 20, 22, 25, or 28 and a primer comprising the sequence of SEQ ID NOs: 21, 23, 26, 29, or 31. The composition can comprise: one or more the signal-generating oligonucleotides, wherein each of the one or more the signal-generating oligonucleotides comprises a sequence selected from the group consisting of SEQ ID NOs: 15, 18-19, 24, 27, 30, and 32, or a sequence that exhibits at least about 85% identity to a sequence selected from the group consisting of SEQ ID NOs: 15, 18-19, 24, 27, 30, and 32. In some embodiments, each of the one or more the signal-generating oligonucleotides comprises a sequence selected from the group consisting of SEQ ID NOs: 15, 18-19, 24, 27, 30, and 32. In some embodiments, each of the one or more the signal-generating oligonucleotides consists of a sequence selected from the group consisting of SEQ ID NOs: 15, 18-19, 24, 27, 30, and 32.

Disclosed herein include compositions for the detection of an Influenza B virus in a sample. In some embodiments, the composition comprises: at least one pair of primers capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity to any one of the sequences of SEQ ID NOs: 36-37 and 41. In some embodiments, the at least one pair of primers capable of hybridizing to the target nucleic acid sequence of an Influenza B virus comprises a primer comprising the sequence of SEQ ID NO: 36 and a primer comprising the sequence of SEQ ID NOs: 37 or 41. The composition can comprise: a primer exhibiting at least about 85% identity to SEQ ID NO: 38. The composition can comprise: one or more the signal-generating oligonucleotides, wherein each of the one or more the signal-generating oligonucleotides comprises a sequence selected from the group consisting of SEQ ID NOs: 39-40 and 42-45, or a sequence that exhibits at least about 85% identity to a sequence selected from the group consisting of SEQ ID NOs: 39-40 and 42-45. In some embodiments, each of the one or more the signal-generating oligonucleotides comprises a sequence selected from the group consisting of SEQ ID NOs: 39-40 and 42-45. In some embodiments, each of the one or more the signal-generating oligonucleotides consists of a sequence selected from the group consisting of SEQ ID NOs: 39-40 and 42-45.

In some embodiments, the signal-generating oligonucleotide comprises a 5′ subdomain and a 3′ subdomain. In some embodiments, the signal-generating oligonucleotide comprises a loop domain situated between the 5′ subdomain and the 3′ subdomain. In some embodiments, intramolecular nucleotide base pairing between the 5′ subdomain and the 3′ subdomain are capable of forming a paired stem domain. In some embodiments, the loop domain comprises one or more polymerase stoppers. In some embodiments, the 5′ subdomain, the paired stem domain, and/or the 3′ subdomain does not comprise the one or more polymerase stoppers. In some embodiments, the signal-generating oligonucleotide comprises a 5′ terminal domain situated 5′ of the 5′ subdomain. In some embodiments, the signal-generating oligonucleotide comprises a 3′ terminal domain situated 3′ of the 5′ subdomain. In some embodiments, the 5′ terminal domain and/or the 3′ terminal domain does not comprise the one or more polymerase stoppers.

The one or more polymerase stoppers can comprise one or more 2′-O-methyl (2′OM) RNA nucleotides. In some embodiments, the one or more polymerase stoppers comprise one or more of an abasic site, a stable abasic site, a chemically trapped abasic site, or any combination thereof. In some embodiments, the stable abasic site comprises 1′,2′-dideoxy. In some embodiments, the chemically trapped abasic site comprises an abasic site reacted with alkoxy amine or sodium borohydride. In some embodiments, the abasic site comprises an apurinic site, an apyrimidinic site, or both. In some embodiments, the abasic site is generated by an alkylating agent or an oxidizing agent. In some embodiments, the one or polymerase stoppers comprise: one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methyl cytidines, one or more 5-hydroxymethylcytidines, one or more 2′-O-Methyl RNA bases, one or more unmethylated RNA bases, one or more Iso-deoxycytidines (Iso-dCs), one or more Iso-deoxyguanosines (Iso-dGs), one or more C3(OC₃H₆OPO₃) groups, one or more photo-cleavable (PC) [OC₃H₆—C(o)NHCH₂—C₆H₃NO₂—CH(CH₃)OPO₃] groups, one or more hexandiol groups, one or more spacer 9 (iSp9) [(OCH₂CH₂)₃OPO₃] groups, one or more spacer 18 (iSp18) [(OCH₂CH₂₆OPO₃] groups, or any combination thereof.

In some embodiments, the signal-generating oligonucleotide comprises one or more phosphorothioate linkages and/or one or more locked nucleic acids. In some embodiments, the signal-generating oligonucleotide is a TaqMan detection probe oligonucleotide, a molecular beacon detection probe oligonucleotide, or a molecular torch detection probe oligonucleotide. In some embodiments, the signal-generating oligonucleotide comprises a label, for example a quenchable label (e.g., a fluorophore). In some embodiments, the signal-generating oligonucleotide comprises a quencher. In some embodiments, the label is situated in the 3′ terminal domain and the quencher is situated in the 5′ terminal domain, and/or the label is situated in the 5′ terminal domain and the quencher is situated in the 3′ terminal domain.

There is provided, in some embodiments, a signal-generating oligonucleotide or primer up to about 100 nucleotides in length which is capable of hybridizing to a target nucleic acid sequence of N. Gonorrhea, wherein said signal-generating oligonucleotide or primer comprises a sequence selected from the group consisting of SEQ ID NOs: 1-3, 12-14, and 16-17, or sequence that exhibits at least about 85% identity to a sequence selected from the group consisting of SEQ ID NOs: 1-3, 12-14, and 16-17. In some embodiments, said signal-generating oligonucleotide or primer consists of a sequence selected from the group consisting of SEQ ID NOs: 1-3, 12-14, and 16-17, or sequence that exhibits at least about 85% identity to a sequence selected from the group consisting of SEQ ID NOs: 1-3, 12-14, and 16-17. In some embodiments, said signal-generating oligonucleotide or primer comprises a sequence selected from the group consisting of SEQ ID NOs: 1-3, 12-14, and 16-17. In some embodiments, said signal-generating oligonucleotide or primer consists of a sequence selected from the group consisting of SEQ ID NOs: 1-3, 12-14, and 16-17.

There is provided, in some embodiments, a signal-generating oligonucleotide or primer up to about 100 nucleotides in length which is capable of hybridizing to a target nucleic acid sequence of C. trachomatis, wherein said signal-generating oligonucleotide or primer comprises a sequence selected from the group consisting of SEQ ID NOs: 15 and 18-32, or sequence that exhibits at least about 85% identity to a sequence selected from the group consisting of SEQ ID NOs: 15 and 18-32. In some embodiments, said signal-generating oligonucleotide or primer consists of a sequence selected from the group consisting of SEQ ID NOs: 15 and 18-32, or sequence that exhibits at least about 85% identity to a sequence selected from the group consisting of SEQ ID NOs: 15 and 18-32. In some embodiments, said signal-generating oligonucleotide or primer comprises a sequence selected from the group consisting of SEQ ID NOs: 15 and 18-32. In some embodiments, said signal-generating oligonucleotide or primer consists of a sequence selected from the group consisting of SEQ ID NOs: 15 and 18-32.

There is provided, in some embodiments, a signal-generating oligonucleotide or primer up to about 100 nucleotides in length which is capable of hybridizing to a target nucleic acid sequence of an Influenza B virus, wherein said signal-generating oligonucleotide or primer comprises a sequence selected from the group consisting of SEQ ID NOs: 36-45, or sequence that exhibits at least about 85% identity to a sequence selected from the group consisting of SEQ ID NOs: 36-45. In some embodiments, said signal-generating oligonucleotide or primer consists of a sequence selected from the group consisting of SEQ ID NOs: 36-45, or sequence that exhibits at least about 85% identity to a sequence selected from the group consisting of SEQ ID NOs: 36-45. In some embodiments, said signal-generating oligonucleotide or primer comprises a sequence selected from the group consisting of SEQ ID NOs: 36-45. In some embodiments, said signal-generating oligonucleotide or primer consists of a sequence selected from the group consisting of SEQ ID NOs: 36-45.

There is provided, in some embodiments, a signal-generating oligonucleotide or primer up to about 100 nucleotides in length which is capable of hybridizing to a target nucleic acid sequence of an Influenza A virus, wherein said signal-generating oligonucleotide or primer comprises a sequence selected from the group consisting of SEQ ID NOs: 33-35, or sequence that exhibits at least about 85% identity to a sequence selected from the group consisting of SEQ ID NOs: 33-35. In some embodiments, said signal-generating oligonucleotide or primer consists of a sequence selected from the group consisting of SEQ ID NOs: 33-35, or sequence that exhibits at least about 85% identity to a sequence selected from the group consisting of SEQ ID NOs: 33-35. In some embodiments, said signal-generating oligonucleotide or primer comprises a sequence selected from the group consisting of SEQ ID NOs: 33-35. In some embodiments, said signal-generating oligonucleotide or primer consists of a sequence selected from the group consisting of SEQ ID NOs: 33-35.

There is provided, in some embodiments, compositions comprising two or more of the signal-generating oligonucleotides and/or primers provided herein. The composition can comprise: a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent, and wherein the detergent comprises one or more of a cationic surfactant, an anionic surfactant, a non-ionic surfactant, and an amphoteric surfactant; and/or a reagent composition comprising one or more amplification reagents comprising one or more components for amplifying the target nucleic acid sequence under isothermal amplification conditions. In some embodiments, said one or more components for amplifying comprise: an enzyme having a hyperthermophile polymerase activity capable of generating a nucleic acid amplification product, optionally the enzyme having a hyperthermophile polymerase activity has an amino acid sequence that is at least about 90% identical to the amino acid sequence of SEQ ID NO: 7 or a functional fragment thereof, optionally the enzyme having a hyperthermophile polymerase activity has an amino acid sequence that is at least about 95% identical to the amino acid sequence of SEQ ID NO: 7, further optionally the enzyme having a hyperthermophile polymerase activity is a polymerase comprising the amino acid sequence of SEQ ID NO: 7.

### BRIEF DESCRIPTION OF THE DRAWINGS

FIGS. 1A-1B show a non-limiting exemplary schematic of an isothermal amplification reaction provided herein.

FIGS. 2A-2C depict non-limiting exemplary primers and probes (Molecular Beacons) for an Archaeal Polymerase Amplification (APA)-based Neisseria Gonorrhea assay. FIG. 2A depicts the location of primers and a molecular beacon relative to template. FIG. 2B depicts the molecular beacon of FIG. 2A in an un-bound, hairpin formation. FIG. 2C depicts a non-limiting exemplary protected molecular beacon (wherein the “M” indicates a polymerase stopper, e.g., 2′OM-modified base) in an un-bound, hairpin formation.

FIG. 3 depicts non-limiting exemplary non-specific product formation that can arise with the APA-based Neisseria Gonorrhea assay depicted in FIGS. 2A-2B.

FIG. 4 depicts non-limiting exemplary prevention of non-specific product formation in the APA-based Neisseria Gonorrhea assay depicted in FIGS. 2A-2B with the use of a protected probe (e.g., the protected probe shown in FIG. 2C).

FIGS. 5A-5E depict data showing FluB PB2 Assay non-specific product formation (without 2′OM protection). The FluB PB2 reverse primer was titrated to 600 nM (FIG. 5A), 500 nM (FIG. 5B), 400 nM (FIG. 5C), 300 nM (FIG. 5D), and 200 nM (FIG. 5E).

FIGS. 6A-6D depict data related to prevention of FluB PB2 Assay non-specific product formation via 2′OM-modified beacons. Results generated using non-modified control molecular beacon (FIG. 6A; LNA3.13), 2′OM-modified version 1 (FIG. 6B; LNA3.13 ml), 2′OM-modified version 2 (FIG. 6C; LNA3.13m2), and 2′OM-modified version 3 (FIG. 6D; LNA3.13m3) are depicted.

FIGS. 7A-7D depict data related to false positive assessment in Neisseria Gonorrhea and Chlamydia trachomatis assays conducted with NTC 10% urine samples (FIGS. 7A-7B) and NTC vaginal swab samples (FIGS. 7C-7D) using 8U 9dN polymerase (FIG. 7A, FIG. 7C) and 12U 9dN polymerase (FIG. 7B, FIG. 7D).

FIGS. 8A-8D depict data related to false positive assessment in Chlamydia trachomatis assays (FIGS. 8A-8B) and Neisseria Gonorrhea assays (FIGS. 8C-8D) conducted with NTC 15% urine samples using nominal probes (FIG. 8A, FIG. 8C) and protected probes (FIG. 8B, FIG. 8D). FIG. 8B contains 1 errant amplification associated with a suspected Chlamydia trachomatis contamination event.

FIGS. 9A-9D depict data related to false positive assessment in Chlamydia trachomatis assays (FIGS. 9A-9B) and Neisseria Gonorrhea assays (FIGS. 9C-9D) conducted with vaginal swab samples using nominal probes (FIG. 9A, FIG. 9C) and protected probes (FIG. 9B, FIG. 9D).

FIGS. 10A-10D depict data related to false positive assessment in Chlamydia trachomatis assays (FIGS. 10A-10B) and Neisseria Gonorrhea assays (FIGS. 10C-10D) conducted with NTC urine samples and using protected probes. FIG. 10D contains 1 errant amplification associated with a suspected Neisseria Gonorrhea contamination event.

FIGS. 11A-11B depict data related to false positive assessment in Chlamydia trachomatis assays (FIG. 11A) and Neisseria Gonorrhea assays (FIG. 11B) conducted with NTC vaginal swab samples and using protected probes.

FIG. 12 depicts a non-limiting exemplary protected molecular beacon (wherein the “M” indicates a polymerase stopper, e.g., 2′OM-modified base) in an un-bound, hairpin formation for use in a Chlamydia trachomatis assay.

FIGS. 13A-13C depict data related to RNA base incorporation for protection of molecular beacons in FluA assays. FluA assays were performed with NTC only-Primer Drop (FIGS. 13A-13B) or probes were screened with target (FIG. 13C).

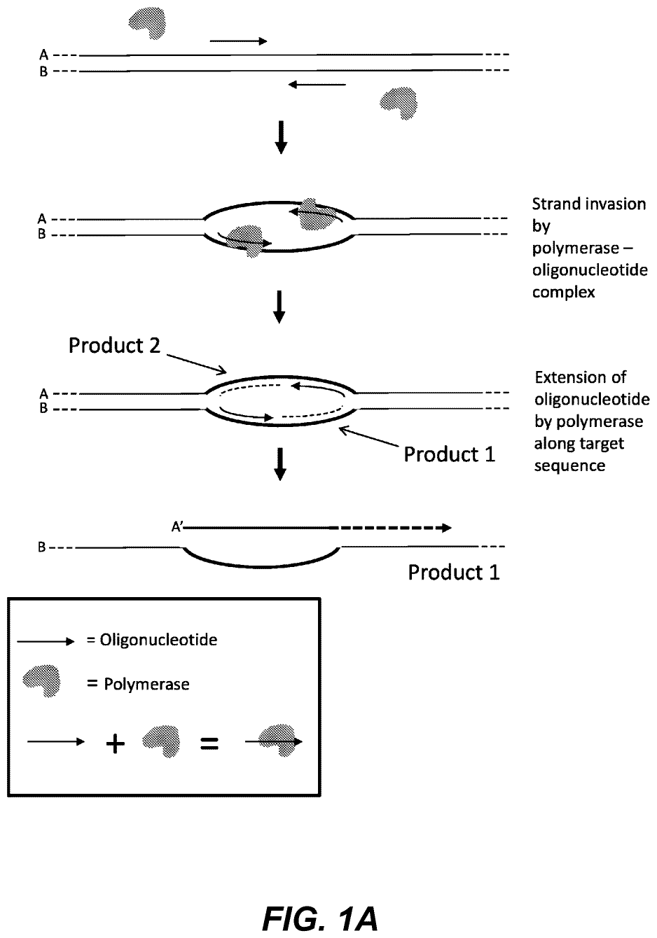

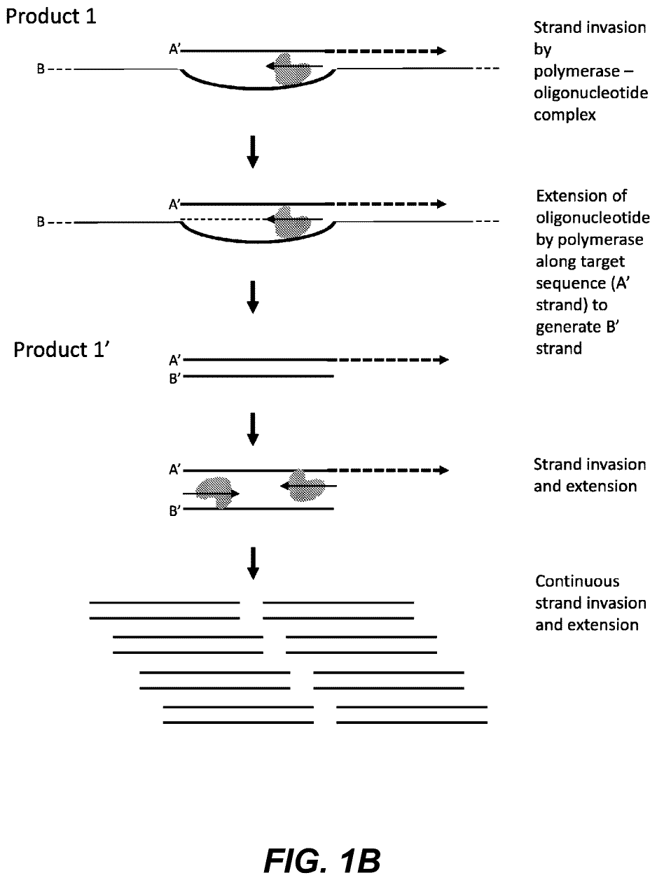

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

Disclosed herein include methods for detecting a target nucleic acid sequence in a sample. In some embodiments, the method comprises: amplifying a target nucleic acid sequence in an amplification reaction mixture under an isothermal amplification condition, thereby generating a nucleic acid amplification product; and detecting the nucleic acid amplification product with a signal-generating oligonucleotide, wherein the signal-generating oligonucleotide is capable of hybridizing to the nucleic acid amplification product and comprises one or more polymerase stoppers. The method can comprise: contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence. The method can comprise: contacting a reagent composition with the treated sample to generate the amplification reaction mixture, wherein the reagent composition comprises one or more amplification reagents.

Disclosed herein include signal-generating oligonucleotides, for example signal-generating oligonucleotides capable of hybridizing to a nucleic acid amplification product. In some embodiments, the signal-generating oligonucleotide comprises a 5′ subdomain and a 3′ subdomain, and optionally a loop domain situated between the 5′ subdomain and the 3′ subdomain. In some embodiments, intramolecular nucleotide base pairing between the 5′ subdomain and the 3′ subdomain are capable of forming a paired stem domain. In some embodiments, the loop domain comprises one or more polymerase stoppers. In some embodiments, the 5′ subdomain, the paired stem domain, and/or the 3′ subdomain does not comprise the one or more polymerase stoppers. In some embodiments, the nucleic acid amplification product is generated by amplifying a target nucleic acid sequence comprising a first strand and a second strand complementary to each other. In some embodiments, the nucleic acid amplification product is generated by amplifying the target nucleic acid sequence with the forward primer and the reverse primer.

Disclosed herein include kits for detecting a target nucleic acid sequence in a sample. In some embodiments, the kit comprises: a signal-generating oligonucleotide disclosed herein. The kit can comprise: a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent. The kit can comprise: a reagent composition comprising one or more amplification reagents comprising one or more components for amplifying the target nucleic acid sequence under isothermal amplification conditions.

Disclosed herein include methods for detecting N. Gonorrhea in a sample. In some embodiments, the method comprises: contacting said sample with at least one pair of primers, wherein said at least one pair of primers is capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity to any one of the sequences of SEQ ID NOs: 2-3 and 16-17. The method can comprise: generating amplicons (e.g., nucleic acid amplification products) of the target nucleic acid sequence, if said sample comprises N. Gonorrhea. The method can comprise: determining the presence or amount of said amplicons as an indication of the presence of N. Gonorrhea in said sample.

Disclosed herein include methods for detecting C. trachomatis in a sample. In some embodiments, the method comprises: contacting said sample with at least one pair of primers, wherein said at least one pair of primers is capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity to any one of the sequences of SEQ ID NOs: 20-23, 25-26, 28-29, and 31. The method can comprise: generating amplicons (e.g., nucleic acid amplification products) of the target nucleic acid sequence, if said sample comprises C. trachomatis. The method can comprise: determining the presence or amount of said amplicons as an indication of the presence of C. trachomatis in said sample.

Disclosed herein include methods for detecting an Influenza B virus in a sample. In some embodiments, the method comprises: contacting said sample with at least one pair of primers, wherein said at least one pair of primers is capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity to any one of the sequences of SEQ ID NOs: 36-37 and 41. The method can comprise: generating amplicons (e.g., nucleic acid amplification products) of the target nucleic acid sequence, if said sample comprises an Influenza B virus. The method can comprise: determining the presence or amount of said amplicons as an indication of the presence of an Influenza B virus in said sample.

Disclosed herein include compositions for the detection of N. Gonorrhea in a sample. In some embodiments, the composition comprises: at least one pair of primers capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity to any one of the sequences of SEQ ID NOs: 2-3 and 16-17.

Disclosed herein include compositions for the detection of C. trachomatis in a sample. In some embodiments, the composition comprises: at least one pair of primers capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity to any one of the sequences of SEQ ID NOs: 20-23, 25-26, 28-29, and 31.

Disclosed herein include compositions for the detection of an Influenza B virus in a sample. In some embodiments, the composition comprises: at least one pair of primers capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity to any one of the sequences of SEQ ID NOs: 36-37 and 41.

There is provided, in some embodiments, a signal-generating oligonucleotide or primer up to about 100 nucleotides in length which is capable of hybridizing to a target nucleic acid sequence of N. Gonorrhea, wherein said signal-generating oligonucleotide or primer comprises a sequence selected from the group consisting of SEQ ID NOs: 1-3, 12-14, and 16-17, or sequence that exhibits at least about 85% identity to a sequence selected from the group consisting of SEQ ID NOs: 1-3, 12-14, and 16-17.

There is provided, in some embodiments, a signal-generating oligonucleotide or primer up to about 100 nucleotides in length which is capable of hybridizing to a target nucleic acid sequence of C. trachomatis, wherein said signal-generating oligonucleotide or primer comprises a sequence selected from the group consisting of SEQ ID NOs: 15 and 18-32, or sequence that exhibits at least about 85% identity to a sequence selected from the group consisting of SEQ ID NOs: 15 and 18-32.

There is provided, in some embodiments, a signal-generating oligonucleotide or primer up to about 100 nucleotides in length which is capable of hybridizing to a target nucleic acid sequence of an Influenza B virus, wherein said signal-generating oligonucleotide or primer comprises a sequence selected from the group consisting of SEQ ID NOs: 36-45, or sequence that exhibits at least about 85% identity to a sequence selected from the group consisting of SEQ ID NOs: 36-45.

There is provided, in some embodiments, a signal-generating oligonucleotide or primer up to about 100 nucleotides in length which is capable of hybridizing to a target nucleic acid sequence of an Influenza A virus, wherein said signal-generating oligonucleotide or primer comprises a sequence selected from the group consisting of SEQ ID NOs: 33-35, or sequence that exhibits at least about 85% identity to a sequence selected from the group consisting of SEQ ID NOs: 33-35. There is provided, in some embodiments, compositions comprising two or more of the signal-generating oligonucleotides and/or primers provided herein.

### Methods and Compositions for Pathogen Detection

As described herein, nucleic acid amplification and detection reactions can be performed to determine the presence, absence, type, and/or level of pathogens in a sample, such as, for example, N. Gonorrhea, C. trachomatis, and/or an Influenza virus (e.g., an Influenza B virus). In some embodiments, the presence, absence and/or level of one or more of N. Gonorrhea, C. trachomatis, and an Influenza virus is determined by detecting one or more target sequences of said target organisms using methods known in the art, such as DNA amplifications. In some embodiments, multiplex reactions can be performed to detect the presence, absence or level for two or more of N. Gonorrhea, C. trachomatis, and an Influenza virus.

There are provided, in some embodiments, methods for detecting N. Gonorrhea in a sample. In some embodiments, the method comprises: contacting said sample with at least one pair of primers, wherein said at least one pair of primers is capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 2-3 and 16-17. The method can comprise: generating amplicons (e.g., nucleic acid amplification products) of the target nucleic acid sequence, if said sample comprises N. Gonorrhea. The method can comprise: determining the presence or amount of said amplicons as an indication of the presence of N. Gonorrhea in said sample. The at least one pair of primers can comprise a first primer comprising the sequence of SEQ ID NOs: 2 or 17, and a second primer comprising the sequence of SEQ ID NOs: 3 or 16. In some embodiments, the at least one pair of primers capable of hybridizing to the target nucleic acid sequence of N. Gonorrhea is SEQ ID NOs: 2 and 3, SEQ ID NOs: 2 and 16, SEQ ID NOs: 17 and 3, or SEQ ID NOs: 17 and 16. Determining the presence or amount of amplicons of the target nucleic acid sequence can comprise contacting the amplicons with one or more the signal-generating oligonucleotides, wherein each of the one or more the signal-generating oligonucleotides comprises a sequence selected from the group consisting of SEQ ID NOs: 1 and 12-14, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 1 and 12-14. Each of the one or more the signal-generating oligonucleotides can comprise, or consist of, a sequence selected from the group consisting of SEQ ID NOs: 1 and 12-14.

There are provided, in some embodiments, methods for detecting C. trachomatis in a sample. In some embodiments, the method comprises: contacting said sample with at least one pair of primers, wherein said at least one pair of primers is capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 20-23, 25-26, 28-29, and 31. The method can comprise: generating amplicons (e.g., nucleic acid amplification products) of the target nucleic acid sequence, if said sample comprises C. trachomatis. The method can comprise: determining the presence or amount of said amplicons as an indication of the presence of C. trachomatis in said sample. The at least one pair of primers can comprise a first primer comprising the sequence of SEQ ID NOs: 20, 22, 25, or 28, and a second primer comprising the sequence of SEQ ID NOs: 21, 23, 26, 29, or 31. In some embodiments, the at least one pair of primers capable of hybridizing to the target nucleic acid sequence of C. trachomatis is SEQ ID NOs: 20 and 21, SEQ ID NOs: 20 and 23, SEQ ID NOs: 20 and 26, SEQ ID NOs: 20 and 29, SEQ ID NOs: 20 and 31, SEQ ID NOs: 22 and 21, SEQ ID NOs: 22 and 23, SEQ ID NOs: 22 and 26, SEQ ID NOs: 22 and 29, SEQ ID NOs: 22 and 31, SEQ ID NOs: 25 and 21, SEQ ID NOs: 25 and 23, SEQ ID NOs: 25 and 26, SEQ ID NOs: 25 and 29, SEQ ID NOs: 25 and 31, SEQ ID NOs: 28 and 21, SEQ ID NOs: 28 and 23, SEQ ID NOs: 28 and 26, SEQ ID NOs: 28 and 29, or SEQ ID NOs: 28 and 31. Determining the presence or amount of amplicons of the target nucleic acid sequence can comprise contacting the amplicons with one or more the signal-generating oligonucleotides, wherein each of the one or more the signal-generating oligonucleotides comprises a sequence selected from the group consisting of SEQ ID NOs: 15, 18-19, 24, 27, 30, and 32, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 15, 18-19, 24, 27, 30, and 32. Each of the one or more the signal-generating oligonucleotides can comprise, or consist of, a sequence selected from the group consisting of SEQ ID NOs: 15, 18-19, 24, 27, 30, and 32.

There are provided, in some embodiments, methods for detecting an Influenza B virus in a sample. In some embodiments, the method comprises: contacting said sample with at least one pair of primers, wherein said at least one pair of primers is capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 36-37 and 41. The method can comprise: generating amplicons (e.g., nucleic acid amplification products) of the target nucleic acid sequence, if said sample comprises an Influenza B virus. The method can comprise: determining the presence or amount of said amplicons as an indication of the presence of an Influenza B virus in said sample. The at least one pair of primers can comprise a first primer comprising the sequence of SEQ ID NO: 36, and a second primer comprising the sequence of SEQ ID NOs: 37 or 41. In some embodiments, the at least one pair of primers capable of hybridizing to the target nucleic acid sequence of an Influenza B virus is SEQ ID NOs: 36 and 37, or SEQ ID NOs: 36 and 41. Determining the presence or amount of amplicons of the target nucleic acid sequence can comprise contacting the amplicons with one or more the signal-generating oligonucleotides, wherein each of the one or more the signal-generating oligonucleotides comprises a sequence selected from the group consisting of SEQ ID NOs: 39-40 and 42-45, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 39-40 and 42-45. Each of the one or more the signal-generating oligonucleotides can comprise, or consist of, a sequence selected from the group consisting of SEQ ID NOs: 39-40 and 42-45. The method can comprise: contacting sample ribonucleic acids of the sample with a reverse transcriptase and a primer exhibiting at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to SEQ ID NO: 38.

In some embodiments, the sample is contacted with a reagent composition comprising the at least one pair of primers to generate the amplification reaction mixture. The method can comprise: contacting the sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence; and contacting a reagent composition comprising the at least one pair of primers with the treated sample to generate the amplification reaction mixture, wherein the reagent composition comprises one or more amplification reagents. Generating amplicons of the target nucleic acid sequence can comprise amplifying the target nucleic acid sequence in the amplification reaction mixture under an amplification condition, thereby generating amplicons of the target nucleic acid sequence. The determining step can comprise contacting the amplicons of the target nucleic acid sequence with the signal-generating oligonucleotide for hybridization, optionally the determining comprises use of a real-time detection method. A label of the signal-generating oligonucleotide can be capable of generating a signal upon the signal-generating oligonucleotide hybridizing the amplicons of the target nucleic acid sequence. In some embodiments, upon the signal-generating oligonucleotide hybridizing the amplicons of the target nucleic acid sequence, the label generates a signal, optionally the signal is fluorescence.

There are provided, in some embodiments, compositions for the detection of N. Gonorrhea in a sample. In some embodiments, the composition comprises: at least one pair of primers capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 2-3 and 16-17. In some embodiments, the at least one pair of primers capable of hybridizing to the target nucleic acid sequence of N. Gonorrhea comprises a primer comprising the sequence of SEQ ID NOs: 2 or 17 and a primer comprising the sequence of SEQ ID NOs: 3 or 16. The composition can comprise: one or more the signal-generating oligonucleotides, wherein each of the one or more the signal-generating oligonucleotides comprises a sequence selected from the group consisting of SEQ ID NOs: 1 and 12-14, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 1 and 12-14. Each of the one or more the signal-generating oligonucleotides can comprise, or consist of, a sequence selected from the group consisting of SEQ ID NOs: 1 and 12-14.

There are provided, in some embodiments, compositions for the detection of C. trachomatis in a sample. In some embodiments, the composition comprises: at least one pair of primers capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 20-23, 25-26, 28-29, and 31. In some embodiments, the at least one pair of primers capable of hybridizing to the target nucleic acid sequence of C. trachomatis comprises a primer comprising the sequence of SEQ ID NOs: 20, 22, 25, or 28 and a primer comprising the sequence of SEQ ID NOs: 21, 23, 26, 29, or 31. The composition can comprise: one or more the signal-generating oligonucleotides, wherein each of the one or more the signal-generating oligonucleotides comprises a sequence selected from the group consisting of SEQ ID NOs: 15, 18-19, 24, 27, 30, and 32, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 15, 18-19, 24, 27, 30, and 32. Each of the one or more the signal-generating oligonucleotides can comprise, or consist of, a sequence selected from the group consisting of SEQ ID NOs: 15, 18-19, 24, 27, 30, and 32.

There are provided, in some embodiments, compositions for the detection of an Influenza B virus in a sample. In some embodiments, the composition comprises: at least one pair of primers capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to any one of the sequences of SEQ ID NOs: 36-37 and 41. In some embodiments, the at least one pair of primers capable of hybridizing to the target nucleic acid sequence of an Influenza B virus comprises a primer comprising the sequence of SEQ ID NO: 36 and a primer comprising the sequence of SEQ ID NOs: 37 or 41. The composition can comprise: a primer exhibiting at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to SEQ ID NO: 38. The composition can comprise: one or more the signal-generating oligonucleotides, wherein each of the one or more the signal-generating oligonucleotides comprises a sequence selected from the group consisting of SEQ ID NOs: 39-40 and 42-45, or a sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 39-40 and 42-45. Each of the one or more the signal-generating oligonucleotides can comprise, or consist of, a sequence selected from the group consisting of SEQ ID NOs: 39-40 and 42-45.

There is provided, in some embodiments, a signal-generating oligonucleotide or primer up to about 100 nucleotides in length which is capable of hybridizing to a target nucleic acid sequence of N. Gonorrhea, wherein said signal-generating oligonucleotide or primer comprises a sequence selected from the group consisting of SEQ ID NOs: 1-3, 12-14, and 16-17, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 1-3, 12-14, and 16-17. In some embodiments, said signal-generating oligonucleotide or primer consists of a sequence selected from the group consisting of SEQ ID NOs: 1-3, 12-14, and 16-17, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 1-3, 12-14, and 16-17. Said signal-generating oligonucleotide or primer can comprise, or consist of, a sequence selected from the group consisting of SEQ ID NOs: 1-3, 12-14, and 16-17.

There is provided, in some embodiments, a signal-generating oligonucleotide or primer up to about 100 nucleotides in length which is capable of hybridizing to a target nucleic acid sequence of C. trachomatis, wherein said signal-generating oligonucleotide or primer comprises a sequence selected from the group consisting of SEQ ID NOs: 15 and 18-32, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 15 and 18-32. In some embodiments, said signal-generating oligonucleotide or primer consists of a sequence selected from the group consisting of SEQ ID NOs: 15 and 18-32, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 15 and 18-32. Said signal-generating oligonucleotide or primer can comprise, or consist of, a sequence selected from the group consisting of SEQ ID NOs: 15 and 18-32.

There is provided, in some embodiments, a signal-generating oligonucleotide or primer up to about 100 nucleotides in length which is capable of hybridizing to a target nucleic acid sequence of an Influenza B virus, wherein said signal-generating oligonucleotide or primer comprises a sequence selected from the group consisting of SEQ ID NOs: 36-45, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 36-45. In some embodiments, said signal-generating oligonucleotide or primer consists of a sequence selected from the group consisting of SEQ ID NOs: 36-45, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 36-45. Said signal-generating oligonucleotide or primer can comprise, or consist of, a sequence selected from the group consisting of SEQ ID NOs: 36-45.

There is provided, in some embodiments, a signal-generating oligonucleotide or primer up to about 100 nucleotides in length which is capable of hybridizing to a target nucleic acid sequence of an Influenza A virus, wherein said signal-generating oligonucleotide or primer comprises a sequence selected from the group consisting of SEQ ID NOs: 33-35, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 33-35. In some embodiments, said signal-generating oligonucleotide or primer consists of a sequence selected from the group consisting of SEQ ID NOs: 33-35, or sequence that exhibits at least about 85% identity (e.g., 85%, 86%, 87%, 88%, 89%, 90%, 91%, 92%, 93%, 94%, 95%, 96%, 97%, 98%, 99%, 100%, or a number or a range between any two of these values) to a sequence selected from the group consisting of SEQ ID NOs: 33-35. Said signal-generating oligonucleotide or primer can comprise, or consist of, a sequence selected from the group consisting of SEQ ID NOs: 33-35.

There is provided, in some embodiments, compositions comprising two or more of the signal-generating oligonucleotides and/or primers provided herein. Also provided herein are oligonucleotides (e.g., amplification primers or signal-generating oligonucleotides) containing 1, 2, 3, 4 or more mismatches or universal nucleotides relative to SEQ ID NOs: 1-45 or the complement thereof, including oligonucleotides that are at least 80% identical (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) to SEQ ID NOs: 1-45 or the complement thereof.

### Protected Probes

Disclosed herein include methods and compositions comprising modified Molecular Beacons (e.g., “protected probes”) that can, in some embodiments, improve assay specificity. The protected probes disclosed herein can be employed in assays employing Archaeal Polymerase Amplification (“APA”) to isothermally amplify a region of interest within a target DNA (or cDNA) template for the purpose of real-time analyte detection. In some embodiments, the protected probes provided herein comprise polymerase stoppers (e.g., one or more 2′-O′Methyl RNA Bases (“2′OM”)) within a Molecular Beacon probe to reduce non-specific product formation (and subsequent false positive signal). Without being bound by any particular theory, in some embodiments, modifying a specific base within the Molecular Beacon construct can prevent unwanted “read through” of the probe molecule. Currently available methods have not leveraged 2′OM bases specifically for this purpose. Spacer modifications (such as C3 spacers) may be used for this same effect, but it has been shown that C3 spacers are not compatible with the Molecular Beacons employed within the APA assays described herein. Without being bound by any particular theory, in some embodiments, the compositions and methods provided herein employ the inability of 9dN (a DNA-dependent, DNA Polymerase) to read RNA template. Specifically, when a 2′OM base (i.e. a methylated RNA base) is encountered within a given template, it is theorized to arrest enzymatic processivity (e.g., the enzyme is unable to successfully “read” this position within the DNA template). 2′-O-Methyl RNA can be found in small RNAs (e.g., tRNA) and is a post-transcriptional modification that is a naturally occurring modification of RNA. Oligonucleotides that contain 2′-O-Methyl RNA can be directly synthesized. This modification can increase the melting temperature of RNA:RNA duplexes while also causing only modest changes in RNA:DNA stability. Additionally, this modification can demonstrate stability with regards to single-stranded ribonuclease attack and susceptibility to DNases is generally 5 to 10-fold less than DNA. The 2′ OM modification can be employed in antisense oligonucleotides for the purpose of improving stability and binding affinity to targets. In addition to the 2′OM modifications described herein, the following alternative base modifications within the context of Molecular Beacons used for APA were also tested: C3 spacer modifications; abasic site modifications; and un-methylated RNA bases. Based on the data generated, 2′OM modifications provide the greatest protection (without rendering the Molecular Beacon incompatible with APA).

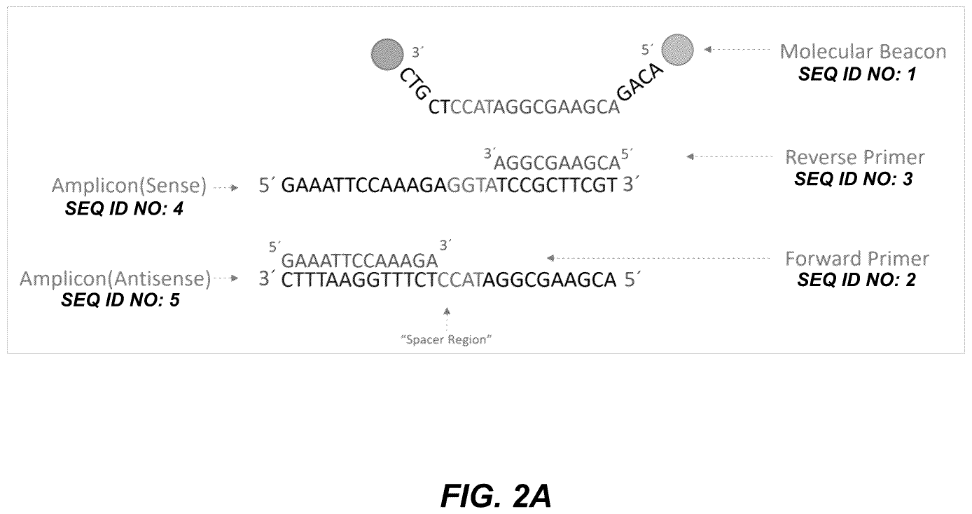

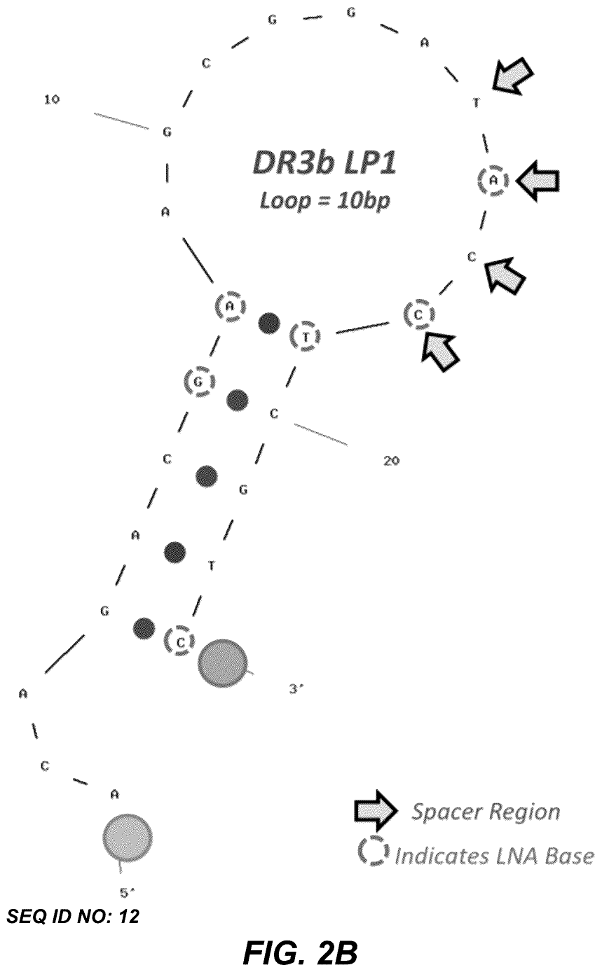

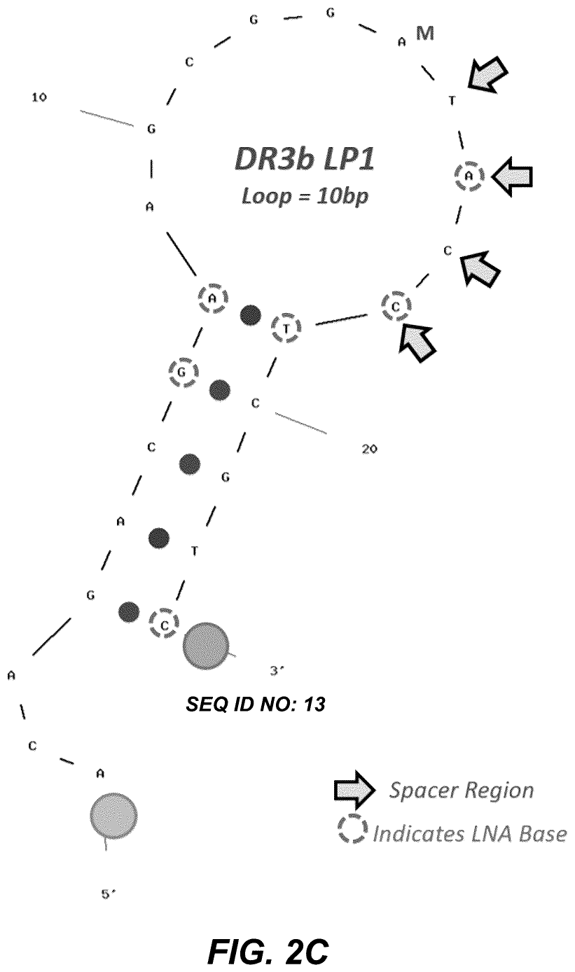

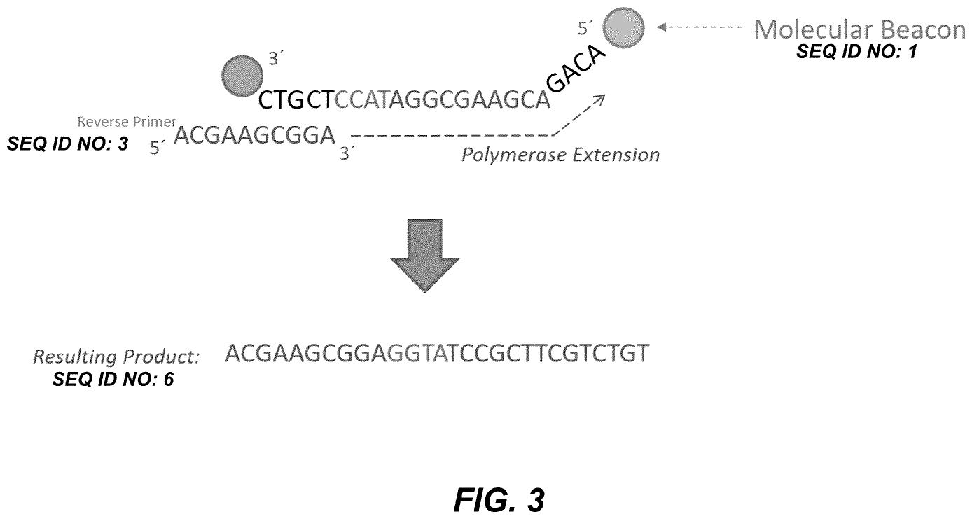

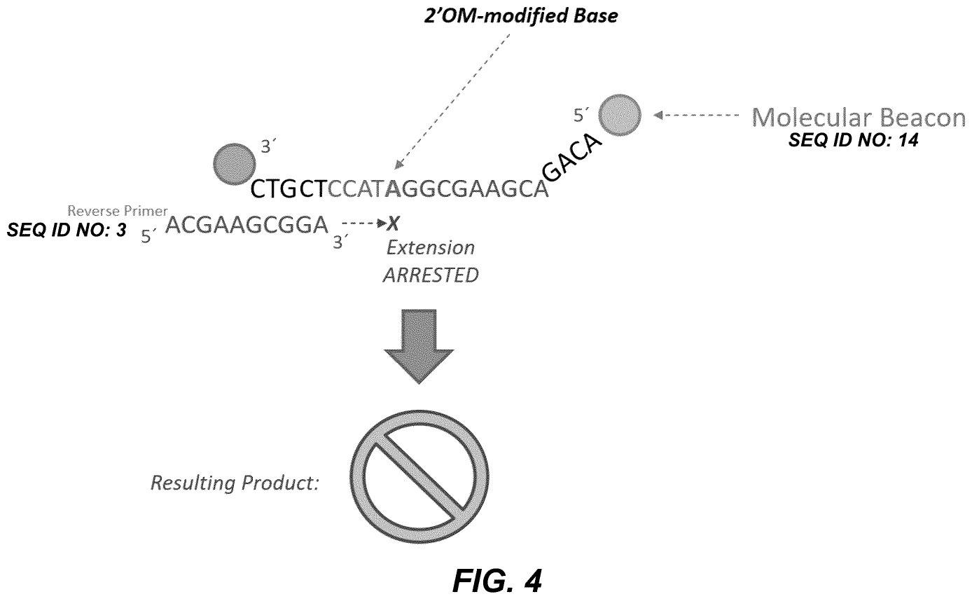

FIGS. 2A-2C depict non-limiting exemplary primers and probes (Molecular Beacon) for the rapid nucleic acid amplification and detection assays provided herein, including an Archaeal Polymerase Amplification (APA)-based Neisseria Gonorrhea assay. FIG. 2A depicts the location of primers and a molecular beacon relative to template. FIG. 2B depicts the molecular beacon of FIG. 2A in an un-bound, hairpin formation. The spacer region is indicated by arrows and corresponds to the region of template situated between the binding sites of the forward and reverse primers. In some embodiments, one or more bases of the probes provided herein is a LNA base (indicated by circles in FIG. 2B). FIG. 2C depicts a protected molecular beacon (wherein the “M” indicates a polymerase stopper, e.g., 2′OM-modified base) in an un-bound, hairpin formation. FIG. 3 depicts non-limiting exemplary non-specific product formation that can arise with the APA-based Neisseria Gonorrhea assay depicted in FIGS. 2A-2B (and without the use of the 2′OM protected probes described herein). An unintentional, non-specific interaction between the reverse primer and the molecular beacon that can theoretically occur is depicted. It is a unique challenge for some embodiments of the APA assay designs provided herein due to the intentional overlap of primer/probe footprints. In this example, the assay employs a Molecular Beacon that entirely overlaps with the Reverse Primer. Polymerase extension of the reverse primer bound to the molecular beacon can yield a product that is capable of exponential amplification via the reverse primer. FIG. 4 depicts non-limiting exemplary prevention of non-specific product formation in the APA-based Neisseria Gonorrhea assay depicted in FIGS. 2A-2B with the use of a protected probe comprising a polymerase stopper as described herein (e.g., the protected probe shown in FIG. 2C). Unintentional, non-specific interaction that can occur between the reverse primer and Molecular Beacon is depicted therein. By preventing the read-through of the embedded primer footprint within the Molecular Beacon, the methods and compositions provided herein employing a protected probe can prevent the formation of the unintended extension product. The 2′OM can serve as the point of control to enable this read-through prevention. Without being bound by any particular theory, the location of this modification determines the level of prevention of non-specific product formation—it is positioned at the very start of the primer-overlap region, to arrest read-through right at the start (i.e., so that no overlap bases are copied). In some embodiments, the one or more polymerase stoppers are located near or at the 3′ end of the first region (immediately adjacent to the spacer region) and within the loop domain.

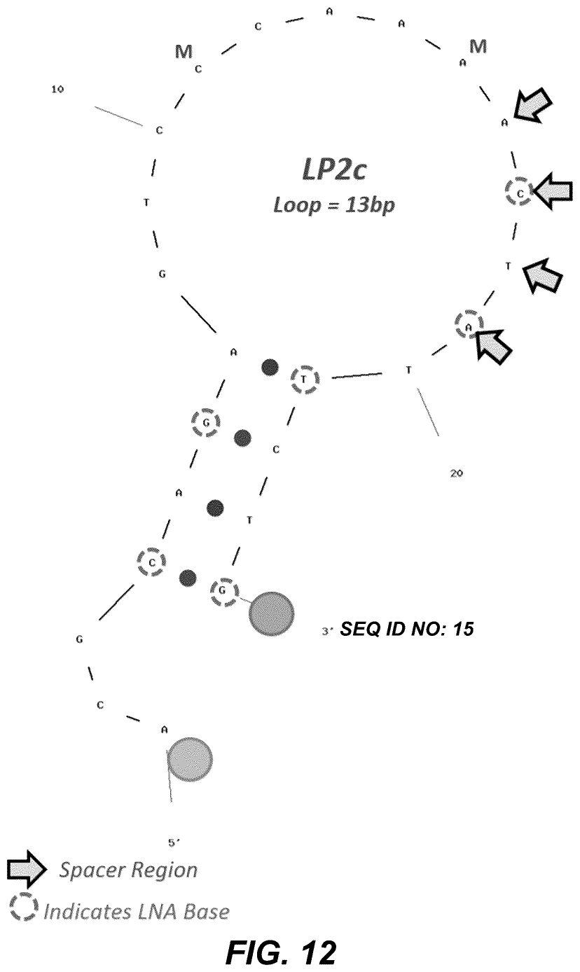

Unintended interaction between the signal-generating oligonucleotide (e.g., molecular beacon) and forward and/or reverse primers leading to non-specific product formation can be a unique and significant challenge for some embodiments of the amplification/detection assays provided herein due to the intentional overlap of primer/probe footprints—the signal-generating oligonucleotide (e.g., molecular beacon) can comprise a first region comprising the sequence of at least a portion of the reverse primer and/or a second region comprising a sequence complementary to at least a portion of the forward primer. Additionally, due to the hairpin nature of signal-generating oligonucleotides provided herein, a repetition of two 3′ terminal nucleotides of the reverse primer in the stem loop can yield unintended reverse primer/probe interaction. Thus, these inherent elements of some of the signal-generating oligonucleotide-based amplification/detection assays provided herein can yield non-specific product formation (and thereby false positive signals). However, the methods and compositions provided herein solve these problems in the art and yield assays with reduced non-specific product formation, reduced false positive signals, and/or increased likelihood of an accurate determination of the presence, absence and/or amount of a target nucleic acid sequence in a sample. The sequences of Neisseria Gonorrhea assay primers and probes described above shown below in Table 1. Additionally, FIG. 12 and Table 2 provide sequences of Chlamydia trachomatis assay primers and protected probes. Table 3 provides non-limiting exemplary FluA and FluB assay primers and probes. In some embodiments, the probes (e.g., molecular beacons) provided herein comprise a 5′ modification (e.g., 5TEX615, FAM). In some embodiments, the probes (e.g., molecular beacons) provided herein comprise a 3′ modification (e.g., 3IAbRQSp, IBFQ).

TABLE 1
Neisseria Gonorrhea (“NG”) Assay Primers and Probes

| | | SEQ ID |
|---|---|---|
| Name | Sequence (5′-3′) | NO |
| Molecular Beacon (DR3b LP1) | ACAGACGAAGCGGATACCTCGTC | 1 |
| Molecular Beacon (DR3b LP1) | acagAC+G+AAGCGGAT+AC+C+TCgT+c | 12 |
| Reverse Primer | ACGAAGCGGA | 3 |
| Reverse Primer | ACGAAGCGG*A | 16 |
| Forward Primer | GAAATTCCAAAGA | 2 |
| Forward Primer | GAAATTCCAAAG*A | 17 |
| Amplicon (sense) | GAAATTCCAAAGAGGTATCCGCTTCGT | 4 |
| Amplicon (anti-sense) | ACGAAGCGGATACCTCTTTGGAATTTC | 5 |
| Non-specific Product | ACGAAGCGGAGGTATCCGCTTCGTCTGT | 6 |
| Protected Molecular Beacon | ACAGACGAAGCGGmATACCTCGTC | 14 |
| Protected Molecular Beacon | ACAGAC+G+AAGCGGmAT+AC+C+TCGT+C | 13 |

m indicates (precedes) 2′ O-Methyl base (e.g. mA, mG, mC, mU)Underline indicates Spacer Region
* indicates phosphorothioated DNA bases (e.g. A*, G*, C*, T*)
* denotes phosphorothioate linkage between adjacent bases
+ indicates locked nucleic acids (e.g. +A, +G, +C, +T)

TABLE 2
Chlamydia trachomatis (“CT”) Assay Primers and Probes

| | | SEQ |
|---|---|---|
| Name | Sequence (5′-3′) | ID NO |
| Molecular Beacon (Ct LP2c) | acg+CA+GAGTCCCAAAA+CT+AT+TCt+G | 18 |
| Molecular Beacon (Ct LP2c (m1)) | acg+CA+GAGTCCCAAmAA+CT+AT+TCt+G | 19 |
| Molecular Beacon (Ct LP2c (m2)) | acg+CA+GAGTCmCCAAmAA+CT+AT+TCt+G | 15 |
| Forward primer (CT F13 PS 2.4) | TTCTTCATC*CG*AA | 20 |
| Reverse primer (CT R7 PS2) | CAGAGTCCCA*AA | 21 |
| Forward Primer (Ct pmpA F2) | CCAACAGGAG*A | 22 |
| Reverse Primer (Ct pmpA R1) | ATTGGATT*CAAG | 23 |
| Molecular Beacon (Ct | cgCCAACAG+GAGAA+TA+T+GT+CCTg+t | 24 |
| pmpA_Hz1) | | |
| Forward Primer (Ct pmpA F2b) | CCGTTCCT*AA | 25 |
| Reverse Primer (Ct pmpA R1b) | TGTGCTCC*ATA | 26 |
| Molecular Beacon (Ct | acagC+CGTTCCTAA+T+T+C+CTacGG+c | 27 |
| pmpA_Hz1b) | | |
| Forward Primer (Ct ORF5 F1a) | TCTGGGAGC*A | 28 |
| Reverse Primer (Ct ORF5 R1a) | GCTGAGACTA*A | 29 |
| Molecular Beacon (Ct | gaacc+gTCTGGGAGCA+T+GTT+CTT+gacg+g | 30 |
| 5ORF_Bcn1a) | | |
| Reverse Primer (CT R13 PS2) | TCAGAGTCCCA*AA | 31 |
| Molecular Beacon (Ct LP3a (m1)) | ACAGC+A+GAGTCCCAAmA+A+CT+ATCTG+C | 32 |

* indicates phosphorothioated DNA bases (e.g. A*, G*, C*, T*)
* denotes phosphorothioate linkage between adjacent bases
m indicates (precedes) 2′ O-Methyl base (e.g. mA, mG, mC, mU)
+ precedes locked nucleic acids (e.g. +A, +G, +C, +T)
Underline indicates Spacer Region

TABLE 3
Flu Assay Primers and Probes

| | | |
|---|---|---|
| Name | Sequence (5′-3′) | SEQ ID NO |

FluA

| | | |
|---|---|---|
| Molecular Beacon (A PB2.2 | agcca+G+CGACCAAAA+G+AATT+gc+t | 33 |
| LP3a) | | |
| Target probe (A PB2.2 LP2) | accgcC+C+ATCCGAAT+T+CT+Tgg+g | 34 |
| Target probe (A PB2.2 LP1b) | acgaG+C+CATCCGAAT+T+CT+Tgg+c | 35 |

FluB

| | | |
|---|---|---|
| Forward primer (B F15 PS2) | GAACGGAACAATA*CA | 36 |
| Reverse primer (B R12 PS2) | CCCATATTCC*AA | 37 |
| RT primer (B rdF17) | rGrGrGrArArCGGAACAATA*CA | 38 |
| Molecular Beacon (B LNA3.13) | agcaC+C+CATATTCCAAT+CTT+C+gg+g | 39 |
| Molecular Beacon (B LPa) | cgcaC+C+CATATTCCAAT+CT+TC+ggg+t | 40 |

* indicates phosphorothioated DNA bases (e.g. A*, G*, C*, T*)
* denotes phosphorothioate linkage between adjacent bases
+ precedes locked nucleic acids (e.g. +A, +G, +C, +T)

There are provided, in some embodiments, methods for detecting a target nucleic acid sequence in a sample. In some embodiments, the method comprises: amplifying a target nucleic acid sequence in an amplification reaction mixture under an isothermal amplification condition, thereby generating a nucleic acid amplification product; and detecting the nucleic acid amplification product with a signal-generating oligonucleotide, wherein the signal-generating oligonucleotide is capable of hybridizing to the nucleic acid amplification product and comprises one or more polymerase stoppers. The method can comprise: contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence. The method can comprise: contacting a reagent composition with the treated sample to generate the amplification reaction mixture, wherein the reagent composition comprises one or more amplification reagents.

There are provided, in some embodiments, signal-generating oligonucleotides. In some embodiments, the signal-generating oligonucleotide is capable of hybridizing to a nucleic acid amplification product. In some embodiments, the signal-generating oligonucleotide comprises a 5′ subdomain and a 3′ subdomain. In some embodiments, the signal-generating oligonucleotide comprises a loop domain situated between the 5′ subdomain and the 3′ subdomain. In some embodiments, intramolecular nucleotide base pairing between the 5′ subdomain and the 3′ subdomain are capable of forming a paired stem domain. In some embodiments, the loop domain comprises one or more polymerase stoppers. In some embodiments, the 5′ subdomain, the paired stem domain, and/or the 3′ subdomain does not comprise the one or more polymerase stoppers. In some embodiments, the nucleic acid amplification product is generated by amplifying a target nucleic acid sequence comprising a first strand and a second strand complementary to each other. In some embodiments, the nucleic acid amplification product is generated by amplifying the target nucleic acid sequence with the forward primer and the reverse primer.

The signal-generating oligonucleotide can comprise a 5′ subdomain and a 3′ subdomain. The signal-generating oligonucleotide can comprise a loop domain situated between the 5′ subdomain and the 3′ subdomain. Intramolecular nucleotide base pairing between the 5′ subdomain and the 3′ subdomain can be capable of forming a paired stem domain. The one or more polymerase stoppers can be situated in the loop domain. In some embodiments, the 5′ subdomain, the paired stem domain, and/or the 3′ subdomain does not comprise the one or more polymerase stoppers.

The nucleic acid amplification product can comprise: (1) the sequence of a forward primer, and the reverse complement thereof, (2) the sequence of a reverse primer, and the reverse complement thereof, and (3) a spacer sequence flanked by (1) the sequence of the forward primer and the reverse complement thereof and (2) the sequence of the reverse primer and the reverse complement thereof. The spacer sequence can be 1 to 10 bases long.

The signal-generating oligonucleotide can comprise a first region comprising the sequence of at least a portion of the reverse primer. The signal-generating oligonucleotide can comprise a second region comprising a sequence complementary to at least a portion of the forward primer. The signal-generating oligonucleotide can comprise a spacer region comprising the sequence of at least a portion of the spacer sequence. The first region, the second region, and/or the spacer region can comprise one or more polymerase stoppers. The first region can comprise a sequence complementary to at least two 3′ terminal nucleotides of the forward primer and/or the reverse primer. The second region can comprise a sequence complementary to at least two 3′ terminal nucleotides of the forward primer and/or the reverse primer. The spacer region can comprise a sequence complementary to at least two 3′ terminal nucleotides of the forward primer and/or the reverse primer. The first region can comprise a sequence complementary to at least two 3′ terminal nucleotides of the forward primer and/or the reverse primer. The second region can comprise a sequence complementary to at least two 3′ terminal nucleotides of the forward primer and/or the reverse primer. The spacer region can comprise a sequence complementary to at least two 3′ terminal nucleotides of the forward primer and/or the reverse primer.

The signal-generating oligonucleotide can be about 10 nucleotides to about 100 nucleotides in length. The forward primer and/or the reverse primer can be about 5 nucleotides to about 25 nucleotides in length. The second region, the spacer region, and/or the first region can be about 1 nucleotide to about 25 nucleotides in length. The 5′ subdomain, the 3′ subdomain, the loop domain, the 5′ terminal domain, and/or the 3′ terminal domain can be about 1 nucleotide to about 25 nucleotides in length. The signal-generating oligonucleotide can comprise a 5′ terminal domain situated 5′ of the 5′ subdomain. The signal-generating oligonucleotide can comprise a 3′ terminal domain situated 3′ of the 5′ subdomain. In some embodiments, the 5′ terminal domain and/or the 3′ terminal domain does not comprise the one or more polymerase stoppers.

The first region can comprise at least a portion of the 5′ subdomain and/or loop domain. The spacer region can comprise at least a portion of the loop domain. The second region can comprise at least a portion of the loop domain and/or 3′ subdomain. The 5′ subdomain can comprise at least a portion of the first region and/or spacer region. The loop domain can comprise at least a portion of the spacer region, the first region, and/or the second region. The 3′ subdomain can comprise at least a portion of the second region and/or spacer region. In some embodiments, the one or more polymerase stoppers are located near or at the 3′ end of the first region (immediately adjacent to the spacer region) and within the loop domain.

As used herein, a “polymerase stopper” is a molecule (e.g., a modified nucleotide) capable of terminating or inhibiting polymerization. In some embodiments, at least one of the one or more polymerase stoppers is a 2′-O-methylated nucleotide. Non-limiting examples of 2′-o methylated nucleotides include 2′-O-methyluridine, 2′-O-methyladenosine, 2′-O-methylcytidine, and 2′-O-methylguanosine. The one or more polymerase stoppers can comprise one or more 2′-O-methyl (2′OM) RNA nucleotides. The one or more polymerase stoppers can comprise one or more of an abasic site, a stable abasic site, a chemically trapped abasic site, or any combination thereof. The stable abasic site can comprise 1′,2′-dideoxy. The chemically trapped abasic site can comprise an abasic site reacted with alkoxy amine or sodium borohydride. The abasic site can comprise an apurinic site, an apyrimidinic site, or both. The abasic site can be generated by an alkylating agent or an oxidizing agent. In some embodiments, the one or polymerase stoppers comprise: one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methyl cytidines, one or more 5-hydroxymethylcytidines, one or more 2′-O-Methyl RNA bases, one or more unmethylated RNA bases, one or more Iso-deoxycytidines (Iso-dCs), one or more Iso-deoxyguanosines (Iso-dGs), one or more C3(OC₃H₆OPO₃) groups, one or more photo-cleavable (PC) [OC₃H₆—C(o)NHCH₂—C₆H₃NO₂—CH(CH₃)OPO₃] groups, one or more hexandiol groups, one or more spacer 9 (iSp9) [(OCH₂CH₂)₃OPO₃] groups, one or more spacer 18 (iSp18) [(OCH₂CH₂₆OPO₃] groups, or any combination thereof. The protected probes provided herein can comprise one or more 2′-O-Methylribonucleosides, such as, for example, 2′-O-methyladenosine, 2′-O-methylcytidine, 2′-O-methylpseudouridine, 2′-O-methylguanosine, 2′-O-methyl-5-methyluridine, and/or 2′-O-methyluridine.

The signal-generating oligonucleotide can comprise one or more phosphorothioate linkages and/or one or more locked nucleic acids. The signal-generating oligonucleotide can be a TaqMan detection probe oligonucleotide, a molecular beacon detection probe oligonucleotide, or a molecular torch detection probe oligonucleotide. The signal-generating oligonucleotide can comprise a label. The label can comprise a quenchable label (e.g., a fluorophore). The signal-generating oligonucleotide can comprise a quencher. The label can be situated in the 3′ terminal domain and the quencher can be situated in the 5′ terminal domain. The label can be situated in the 5′ terminal domain and the quencher can be situated in the 3′ terminal domain.

Upon the forward primer binding the signal-generating oligonucleotide to form a first undesirable duplex, the one or more polymerase stoppers can be capable of stopping polymerase extension of the forward primer of the first undesirable duplex to the 5′ end of the signal-generating oligonucleotide. The one or more polymerase stoppers can be capable of stopping polymerase extension of the forward primer of the first undesirable duplex beyond the one or more polymerase stoppers of the signal-generating oligonucleotide. Upon the reverse primer binding the signal-generating oligonucleotide to form a second undesirable duplex, the one or more polymerase stoppers can be capable of stopping polymerase extension of the reverse primer of the second undesirable duplex to the 5′ end of the signal-generating oligonucleotide. The one or more polymerase stoppers can be capable of stopping polymerase extension of the reverse primer of the second undesirable duplex beyond the one or more polymerase stoppers of the signal-generating oligonucleotide. Upon the extraneous nucleic acid binding the signal-generating oligonucleotide to form a third undesirable duplex, the one or more polymerase stoppers can be capable of stopping polymerase extension of the extraneous nucleic acid of the third undesirable duplex to the 5′ end of the signal-generating oligonucleotide. The one or more polymerase stoppers can be capable of stopping polymerase extension of the extraneous nucleic acid of the third undesirable duplex beyond the one or more polymerase stoppers of the signal-generating oligonucleotide.

The sample nucleic acids can comprise a nucleic acid comprising the target nucleic acid sequence. In some embodiments, amplifying the target nucleic acid sequence comprises: amplifying a target nucleic acid sequence comprising a first strand and a second strand complementary to each other in an isothermal amplification condition, wherein the amplifying comprises contacting a nucleic acid comprising the target nucleic acid sequence with: i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and ii) an enzyme having a hyperthermophile polymerase activity, thereby generating the nucleic acid amplification product. The nucleic acid can be a double-stranded DNA. The nucleic acid can be a product of reverse transcription reaction. The nucleic acid can be a product of reverse transcription reaction generated from sample ribonucleic acids. The amplifying can comprise generating the nucleic acid by a reverse transcription reaction. The sample nucleic acids can comprise sample ribonucleic acids, and the method can comprise contacting sample ribonucleic acids with a reverse transcriptase and/or a reverse transcription primer to generate a cDNA. In some embodiments, amplifying the target nucleic acid sequence comprises: (c1) contacting sample ribonucleic acids with a reverse transcriptase and/or a reverse transcription primer to generate a cDNA; (c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other; (c3) amplifying the target nucleic acid sequence under an isothermal amplification condition, wherein the amplifying comprises contacting the dsDNA with: (i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and (ii) the enzyme having a hyperthermophile polymerase activity, thereby generating the nucleic acid amplification product.

In some embodiments, if the forward primer binds the signal-generating oligonucleotide to form a first undesirable duplex, extension of the forward primer of the first undesirable duplex to the 5′ end of the signal-generating oligonucleotide by an enzyme having a hyperthermophile polymerase activity generates a first undesirable extension product. The first undesirable extension product can be capable of being amplified by the enzyme having a hyperthermophile polymerase activity in the presence of the forward primer and the reverse primer to form a first undesirable amplification product. The one or more polymerase stoppers can be capable of stopping polymerase extension of the forward primer of the first undesirable duplex to generate a first stalled extension product. In some embodiments, the first stalled extension product is not capable of being amplified by the enzyme having a hyperthermophile polymerase activity in the presence of the forward primer and reverse primer to generate the first undesirable amplification product. The one or more polymerase stoppers can be capable of stopping polymerase extension of the forward primer of the first undesirable duplex beyond the one or more polymerase stoppers of the signal-generating oligonucleotide. In some embodiments, if the reverse primer binds the signal-generating oligonucleotide to form a second undesirable duplex, extension of the reverse primer of the second undesirable duplex to the 5′ end of the signal-generating oligonucleotide by an enzyme having a hyperthermophile polymerase activity generates a second undesirable extension product. The second undesirable extension product can be capable of being amplified by the enzyme having a hyperthermophile polymerase activity in the presence of the reverse primer to form a second undesirable amplification product. The one or more polymerase stoppers can be capable of stopping polymerase extension of the reverse primer of the second undesirable duplex to generate a second stalled extension product. In some embodiments, the second stalled extension product is not capable of being amplified by the enzyme having a hyperthermophile polymerase activity in the presence of the reverse primer to generate the second undesirable amplification product. The one or more polymerase stoppers can be capable of stopping polymerase extension of the reverse primer of the second undesirable duplex beyond the one or more polymerase stoppers of the signal-generating oligonucleotide. In some embodiments, if an extraneous nucleic acid binds the signal-generating oligonucleotide to form a third undesirable duplex, extension of the extraneous nucleic acid of the third undesirable duplex to the 5′ end of the signal-generating oligonucleotide by an enzyme having a hyperthermophile polymerase activity generates a third undesirable extension product. The third undesirable extension product can be capable of being amplified by the enzyme having a hyperthermophile polymerase activity in the presence of the reverse primer to form a third undesirable amplification product. The one or more polymerase stoppers can be capable of stopping polymerase extension of the extraneous nucleic acid of the third undesirable duplex to generate a third stalled extension product. In some embodiments, the third stalled extension product is not capable of being amplified by the enzyme having a hyperthermophile polymerase activity in the presence of the reverse primer to generate the third undesirable amplification product. The one or more polymerase stoppers can be capable of stopping polymerase extension of the extraneous nucleic acid of the third undesirable duplex beyond the one or more polymerase stoppers of the signal-generating oligonucleotide.

The detecting step can comprise contacting the nucleic acid amplification product with the signal-generating oligonucleotide for hybridization. Detecting the nucleic acid amplification product can comprise use of a real-time detection method. The label can be capable of generating a signal upon the signal-generating oligonucleotide hybridizing the nucleic acid amplification product. In some embodiments, upon the signal-generating oligonucleotide hybridizing the nucleic acid amplification product, the label generates a signal (e.g., fluorescence). The detecting step can comprise detecting the signal of the label before the amplification reaction, after the amplification reaction, or both. Detecting the nucleic acid amplification product can comprise detecting a signal generated by the label of the signal-generating oligonucleotide. The label can be a fluorophore and the signal can be fluorescence. Detecting a signal can comprise detecting fluorescence emitted by the label. The method can comprise determining the presence, absence and/or amount of the target nucleic acid sequence in the sample. Determining the presence, absence and/or amount of the target nucleic acid sequence in the sample can comprise determining the presence, absence and/or amount of the dsDNA and/or nucleic acid that comprises the target nucleic acid sequence in the sample. In some embodiments, the presence, absence and/or amount of the signal indicates the presence, absence and/or amount of the target nucleic acid sequence in the sample. In some embodiments, the presence, absence and/or amount of the signal indicates the presence, absence and/or amount of the dsDNA and/or nucleic acid that comprises the target nucleic acid sequence in the sample.

The label can be capable of generating a false positive signal upon the signal-generating oligonucleotide hybridizing the first undesirable amplification product, the second undesirable amplification product, and/or the third undesirable amplification product. In some embodiments, upon the signal-generating oligonucleotide hybridizing the first undesirable amplification product, the second undesirable amplification product, and/or the third undesirable amplification product, the label generates a false positive signal. The signal and the false positive signal can be indistinguishable. In some embodiments, the generation of the first undesirable amplification product, the second undesirable amplification product, and/or the third undesirable amplification product reduces the likelihood of an accurate determination of the presence, absence and/or amount of the target nucleic acid sequence in the sample. In some embodiments, the detection of the false positive signal reduces the likelihood of an accurate determination of the presence, absence and/or amount of the target nucleic acid sequence in the sample.

In some embodiments, the presence of the one or more polymerase stoppers in the signal-generating oligonucleotide increases the likelihood of an accurate determination of the presence, absence and/or amount of the target nucleic acid sequence in the sample by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a signal-generating oligonucleotide which does not comprise the one or more polymerase stoppers. In some embodiments, the generation of the first stalled extension product, the second stalled extension product, and/or third stalled extension product does not yield a false positive signal. In some embodiments, the signal-generating oligonucleotide hybridizing the first stalled extension product, the second stalled extension product, and/or the third stalled extension product does not generate a false positive signal. In some embodiments, the nucleic acid amplification product reaches detectable levels at least about 1 minute, about 2 minutes, about 5 minutes, about 10 minutes, about 15 minutes, or about 20 minutes, or a number or a range between any of these values, before the first undesirable amplification product, the second undesirable amplification product, and/or the third undesirable amplification product reaches detectable levels. In some embodiments, the signal reaches detectable levels at least about 1 minute, about 2 minutes, about 5 minutes, about 10 minutes, about 15 minutes, or about 20 minutes, or a number or a range between any of these values, before the false positive signal reaches detectable levels. The appearance of detectable levels of the false positive signal, the first undesirable amplification product, the second undesirable amplification product, and/or the third undesirable amplification product can be delayed by at least about 1 minute, about 2 minutes, about 5 minutes, about 10 minutes, about 15 minutes, or about 20 minutes, or a number or a range between any of these values, as compared to a comparable method wherein the signal-generating oligonucleotide which does not comprise the one or more polymerase stoppers. In some embodiments, the false positive signal, the first undesirable amplification product, the second undesirable amplification product, and/or the third undesirable amplification product does not reach detectable levels for at least about 5 minutes, about 10 minutes, about 15 minutes, or about 20 minutes, after the amplifying step begins. The generation of the false positive signal, the first undesirable amplification product, the second undesirable amplification product, and/or the third undesirable amplification product can be reduced by at least about 1.1-fold (e.g., 1.1-fold, 1.3-fold, 1.5-fold, 1.7-fold, 1.9-fold, 2-fold, 3-fold, 4-fold, 5-fold, 6-fold, 7-fold, 8-fold, 9-fold, 10-fold, or a number or a range between any of these values) as compared to a comparable method wherein the signal-generating oligonucleotide which does not comprise the one or more polymerase stoppers.

Amplifying the target nucleic acid sequence can comprise generating the nucleic acid amplification product at detectable levels within about 20 minutes, about 15 minutes, or about 10 minutes. The detecting can be performed in less than about 20 minutes, less than about 15 minutes, less than about 10 minutes, or less than about 5 minutes, from the time the reagent composition is contacted with the treated sample.

The lysis buffer can comprise one or more of magnesium sulfate, ammonium sulfate, EDTA, and EGTA. The pH of the lysis buffer can be about 1.0 to about 10.0 (e.g., about 2.2). The sample nucleic acids can comprise sample ribonucleic acids and/or sample deoxyribonucleic acids. The sample nucleic acids can comprise cellular RNA, mRNA, microRNA, bacterial RNA, viral RNA, or a combination thereof. In some embodiments, the one or more amplification reagents comprise: a reverse transcriptase; an enzyme having a hyperthermophile polymerase activity; and/or dNTPS. In some embodiments, the enzyme having a hyperthermophile polymerase activity has a reverse transcriptase activity a forward primer; a reverse primer; a reverse transcription primer. The reagent composition can be lyophilized, heat-dried, and/or comprises one or more additives. In some embodiments, the one or more additives comprise: Tween 20, Triton X-100, and/or tween 80; an amino acid; a sugar or sugar alcohol; and/or a polymer. The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof Contacting the reagent composition with the treated sample can comprise dissolving the reagent composition in the treated sample. In some embodiments, the one or more lytic reagents comprise: about 0.001% (w/v) to about 1.0 (w/v) of the treated sample (e.g., about 0.2% (w/v) of the treated sample); and/or a detergent (e.g., one or more of a cationic surfactant, an anionic surfactant, a non-ionic surfactant, and an amphoteric surfactant).

In some embodiments, the method: is performed in a single reaction vessel; does not comprise using any enzymes other than the reverse transcriptase and the enzyme having a hyperthermophile polymerase activity; does not comprise using any enzyme other than the enzyme having a hyperthermophile polymerase activity; does not comprise heat denaturing and/or enzymatic denaturing the nucleic acid during the amplification step; and/or does not comprise contacting the nucleic acid with a single-stranded DNA binding protein.

The target nucleic acid sequence can comprise a length of no longer than about 20 nucleotides to no longer than about 90 nucleotides (e.g., about 30 nucleotides). The forward primer, the reverse primer, and/or the reverse transcription primer can be about 8 to 16 bases long. The nucleic acid amplification product can be about 20 to 40 bases long. The spacer sequence can comprise a portion of the target nucleic acid sequence. The spacer sequence can be 1 to 10 bases long. The isothermal amplification condition can comprise a constant temperature of about 30 °C to about 72 °C, optionally about 55 °C to about 75 °C, optionally about 56 °C to about 67 °C. The amplifying can be performed: for a period of about 5 minutes to about 60 minutes (e.g., a period of about 15 minutes). The amplifying can be performed: in helicase-free, single-stranded binding protein-free, cleavage agent-free, and recombinase-free, isothermal amplification conditions. The amplifying can be carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA). The PCR can be real-time PCR and/or quantitative real-time PCR (QRT-PCR).

The enzyme having a hyperthermophile polymerase activity has an amino acid sequence that can be at least about 90% identical to the amino acid sequence of SEQ ID NO: 7 or a functional fragment thereof. The enzyme having a hyperthermophile polymerase activity has an amino acid sequence that can be at least about 95% identical to the amino acid sequence of SEQ ID NO: 7. The enzyme having a hyperthermophile polymerase activity can be a polymerase comprising the amino acid sequence of SEQ ID NO: 7. In some embodiments, the enzyme having a hyperthermophile polymerase activity has low or no exonuclease activity. The sample ribonucleic acids can be contacted with the reverse transcriptase and the enzyme having a hyperthermophile polymerase activity simultaneously. The sample ribonucleic acids can be contacted with the reverse transcriptase, the enzyme having a hyperthermophile polymerase activity, and the forward and reverse primers simultaneously. The sample ribonucleic acids can be contacted with the reverse transcriptase, the enzyme having a hyperthermophile polymerase activity, the forward primer, the reverse primer, and the reverse transcription primer simultaneously.

In some embodiments, the amplifying comprises and/or does not comprise one or more of the following amplification methods: APA, LAMP, HDA, RPA, SDA, NASBA, TMA, NEAR, RCA, MDA, RAM, cHDA, SPIA, SMART, 3SR, GEAR and IMDA. In some embodiments, the amplifying does not comprise LAMP.

In some embodiments, the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

The term “isothermal amplification reaction” shall be given its ordinary meaning and shall also include reactions wherein the temperature does not significantly change during the reaction. In some embodiments, the temperature of the isothermal amplification reaction does not deviate by more than 10 °C, for example by not more than 5 °C or by not more than 2 °C during the main enzymatic reaction step where amplification takes place. Depending on the method of isothermal amplification of nucleic acids, different enzymes can be used for amplification. Isothermal amplification compositions and methods are described in PCT Application published as WO2017176404, the content of which is incorporated herein by reference in its entirety.

In some embodiments, the methods and components described herein comprise a storage-stable lysis buffer. In some embodiments, the lysis buffer is resistant to the formation of a precipitate for a period of time under a storage condition (e.g., storage-stable lysis buffer). Compositions, kits, and methods wherein lysis buffers resist precipitation are described in the International Application No. PCT/US23/61980 entitled “NON-OPAQUE LYTIC BUFFER COMPOSITION FORMULATIONS” and filed on Feb. 3, 2023, the content of which is incorporated herein by reference in its entirety.

Some embodiments of the methods and compositions provided herein do not comprise agents and/or conditions that denature nucleic acids (e.g., promote strand separation and/or promote unwinding) other than acids and/or low pH conditions. Compositions, kits, and methods for nucleic acid detection wherein nucleic acid strands are dissociated under low pH conditions (e.g., via contact with an acidic lysis buffer) to facilitate subsequent rapid amplification and detection are described in the International Application No. PCT/US23/61978 entitled “METHOD FOR SEPARATING GENOMIC DNA FOR AMPLIFICATION OF SHORT NUCLEIC ACID TARGETS” and filed on Feb. 3, 2023, the content of which is incorporated herein by reference in its entirety.

In some embodiments, the methods and compositions described herein can comprise a lysis buffer and/or a reagent composition. Lysis buffers comprising a lytic agent and a reducing agent, and reagent compositions comprising amplification agents and one or more protectants (e.g., cyclodextrin compounds) capable of sequestering lytic agents, are described in the International Application No. PCT/US22/21015 entitled “ISOTHERMAL AMPLIFICATION OF PATHOGENS” and filed on Mar. 18, 2022, the content of which is incorporated herein by reference in its entirety.

Some embodiments of the methods and compositions described herein can, in some embodiments, be employed in concert with the systems, methods, compositions, and kits for monitoring an amplification reaction described in the U.S. Provisional Patent Application No. 63/374,835 entitled “HAIRPIN INTERNAL CONTROL FOR ISOTHERMAL NUCLEIC ACID AMPLIFICATION” and filed on Sep. 7, 2022, the content of which is incorporated herein by reference in its entirety.

Some embodiments of the methods and compositions described herein can comprise probe(s) melting at temperatures different than the optimal APA reaction temperature to enable multiplexing targets and/or an internal control(s). Compositions, kits, and methods for multiplexed nucleic acid detection are described in the U.S. Provisional Patent Application No. 63/374,831 entitled “ARCHEAL POLYMERASE AMPLIFICATION” and filed on Sep. 7, 2022, the content of which is incorporated herein by reference in its entirety.

### Nucleic Acid, Subjects, Samples and Nucleic Acid Processing

Provided herein are methods and compositions for amplifying nucleic acid. The terms “nucleic acid” and “nucleic acid molecule” may be used interchangeably herein. The terms refer to nucleic acids of any composition, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid can be, or can be from, a plasmid, phage, autonomously replicating sequence (ARS), centromere, artificial chromosome, chromosome, or other nucleic acid able to replicate or be replicated in vitro or in a host cell, a cell, a cell nucleus, a mitochondria, or cytoplasm of a cell. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid may be used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame, “forward” strand or “reverse” strand) and double-stranded polynucleotides. The term “gene” means the segment of DNA involved in producing a polypeptide chain; and generally includes regions preceding and following the coding region (leader and trailer) involved in the transcription/translation of the gene product and the regulation of the transcription/translation, as well as intervening sequences (introns) between individual coding segments (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

In some embodiments of the methods provided herein, one or more nucleic acid targets are amplified. Target nucleic acids may be referred to as target sequences, target polynucleotides, and/or target polynucleotide sequences, and may include double-stranded and single-stranded nucleic acid molecules. Target nucleic acid may be, for example, DNA or RNA. Where the target nucleic acid is an RNA molecule, the molecule may be, for example, double-stranded, single-stranded, or the RNA molecule may comprise a target sequence that is single-stranded. Where the target nucleic acid is double stranded, the target nucleic acid generally includes a first strand and a second strand. A first strand and a second strand may be referred to as a forward strand and a reverse strand and generally are complementary to each other. Where the target nucleic acid is single stranded, a complementary strand may be generated, for example by polymerization and/or reverse transcription, rendering the target nucleic acid double stranded and having a first/forward strand and a second/reverse strand.

A target sequence may refer to either the sense or antisense strand of a nucleic acid sequence, and also may refer to sequences as they exist on target nucleic acids, amplified copies, or amplification products, of the original target sequence. A target sequence can be a subsequence within a larger polynucleotide. For example, a target sequence can be a short sequence (e.g., 20 to 50 bases) within a nucleic acid fragment, a chromosome, a plasmid, that is targeted for amplification. In some embodiments, a target sequence may refer to a sequence in a target nucleic acid that is complementary to an oligonucleotide (e.g., primer) used for amplifying a nucleic acid. Thus, a target sequence may refer to the entire sequence targeted for amplification or may refer to a subsequence in the target nucleic acid where an oligonucleotide binds. An amplification product may be a larger molecule that comprises the target sequence, as well as at least one other sequence, or other nucleotides. The amplification product can be about the same length as the target sequence, for example exactly the same length as the target sequence. The amplification product can comprise, or consist of, the target sequence.

The length of the target sequence, and/or the guanosine cytosine (GC) concentration (percent), may depend, in part, on the temperature at which an amplification reaction is run, and this temperature may depend, in part, on the stability of the polymerase(s) used in the reaction. Sample assays may be performed to determine an appropriate target sequence length and GC concentration for a set of reaction conditions. For example, where a polymerase is stable up to 60 °C to 65 °C, then the target sequence may be, for example, from 19 to 50 nucleotides in length, or for example, from about 40 to 50, 20 to 45, 20 to 40, or 20 to 30 nucleotides in length. GC concentration under these conditions may be, for example, less than 60%, less than 55%, less than 50%, or less than 45%.

Target nucleic acid can include, for example, genomic nucleic acid, plasmid nucleic acid, mitochondrial nucleic acid, cellular nucleic acid, extracellular nucleic acid, bacterial nucleic acid and viral nucleic acid. In some embodiments, target nucleic acid may include genomic DNA, chromosomal DNA, plasmid DNA, mitochondrial DNA, a gene, any type of cellular RNA, messenger RNA, bacterial RNA, viral RNA or a synthetic oligonucleotide. Genomic nucleic acid can include any nucleic acid from any genome, for example, animal, plant, insect, viral and bacterial genomes (e.g., genomes present in spores). In some embodiments, genomic target nucleic acid is within a particular genomic locus or a plurality of genomic loci. A genomic locus can include any or a combination of open reading frame DNA, non-transcribed DNA, intronic sequences, extronic sequences, promoter sequences, enhancer sequences, flanking sequences, or any sequences considered associated with a given genomic locus.

The target sequence can comprise one or more repetitive elements (e.g., multiple repeat sequences, inverted repeat sequences, palindromic sequences, tandem repeats, microsatellites, minisatellites, and the like). In some embodiments, a target sequence is present within a sample nucleic acid (e.g., within a nucleic acid fragment, a chromosome, a genome, a plasmid) as a repetitive element (e.g., a multiple repeat sequence, an inverted repeat sequence, a palindromic sequence, a tandem repeat, a microsatellite repeat, a minisatellite repeat and the like). For example, a target sequence may occur multiple times as a repetitive element and one, some, or all occurrences of the target sequence within a repetitive element may be amplified (e.g., using a single pair of primers) using methods described herein. In some embodiments, a target sequence is present within a sample nucleic acid (e.g., within a nucleic acid fragment, a chromosome, a genome, a plasmid) as a duplication and/or a paralog.

Target nucleic acid can include microRNAs. MicroRNAs, miRNAs, or small temporal RNAs (stRNAs) are short (e.g., about 21 to 23 nucleotides long) and single-stranded RNA sequences involved in gene regulation. MicroRNAs may interfere with translation of messenger RNAs and are partially complementary to messenger RNAs. Target nucleic acid can include microRNA precursors such as primary transcript (pri-miRNA) and pre-miRNA stem-loop-structured RNA that is further processed into miRNA. Target nucleic acid can include short interfering RNAs (siRNAs), which are short (e.g., about 20 to 25 nucleotides long) and at least partially double-stranded RNA molecules involved in RNA interference (e.g., down-regulation of viral replication or gene expression).

Nucleic acid utilized in methods described herein can be obtained from any suitable biological specimen or sample, e.g., isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female, and a subject may be any age (e.g., an embryo, a fetus, infant, child, adult).

A sample or test sample can be any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), serum, plasma, urine, aspirate, biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation.

A sample can include samples containing spores, viruses, cells, nucleic acids from prokaryotes or eukaryotes, and/or any free nucleic acid. For example, a method described herein can be used for detecting nucleic acid on the outside of spores (e.g., without the need for lysis). A sample can be isolated from any material suspected of containing a target sequence, such as from a subject described above. In some embodiments, a target sequence is present in air, plant, soil, or other materials suspected of containing biological organisms.

Nucleic acid can be derived (e.g., isolated, extracted, purified) from one or more sources by methods known in the art. Any suitable method can be used for isolating, extracting and/or purifying nucleic acid from a biological sample, including methods of DNA preparation in the art, and various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), GFX™ Genomic Blood DNA Purification Kit (Amersham, Piscataway, N.J.), and the like or combinations thereof. U.S. Pat. No. 7,888,006 provides DNA purification methods and does not disclose the compositions (e.g., lysis buffers, protectants) and methods provided herein

In some embodiments, a cell lysis procedure is performed. Cell lysis can be performed prior to initiation of an amplification reaction described herein (e.g., to release DNA and/or RNA from cells for amplification). Cell lysis procedures and reagents are known in the art and may be performed by chemical (e.g., detergent, hypotonic solutions, enzymatic procedures, and the like, or combination thereof), physical (e.g., French press, sonication, and the like), or electrolytic lysis methods. For example, chemical methods generally employ lysing agents to disrupt cells and extract nucleic acids from the cells, followed by treatment with chaotropic salts. In some embodiments, cell lysis comprises use of detergents (e.g., ionic, nonionic, anionic, zwitterionic). In some embodiments, cell lysis comprises use of ionic detergents (e.g., sodium dodecyl sulfate (SDS), sodium lauryl sulfate (SLS), deoxycholate, cholate, sarkosyl). Physical methods such as freeze/thaw followed by grinding, the use of cell presses and the like also may be useful. High salt lysis procedures also may be used. For example, an alkaline lysis procedure may be utilized. The latter procedure traditionally incorporates the use of phenol-chloroform solutions, and an alternative phenol-chloroform-free procedure involving three solutions may be utilized. In the latter procedures, one solution can contain 15 mM Tris, pH 8.0; 10 mM EDTA and 100 μg/ml Rnase A; a second solution can contain 0.2N NaOH and 1% SDS; and a third solution can contain 3M KOAc, pH 5.5, for example. In some embodiments, a cell lysis buffer is used in conjunction with the methods and components described herein.

Nucleic acid can be provided for conducting methods described herein without processing of the sample(s) containing the nucleic acid. For example, nucleic acid can be provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components.

Nucleic acid may be provided for conducting methods described herein without modifying the nucleic acid. Modifications can include, for example, denaturation, digestion, nicking, unwinding, incorporation and/or ligation of heterogeneous sequences, addition of epigenetic modifications, addition of labels (e.g., radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, fluorochromes), and the like. Accordingly, in some embodiments, an unmodified nucleic acid is amplified.

Methods disclosed herein for detecting a target nucleic acid sequence (single-stranded or ds DNA and/or RNA) in a sample can detect a target nucleic acid sequence (e.g., DNA or RNA) with a high degree of sensitivity. In some embodiments, the method can be used to detect a target DNA/RNA present in a sample comprising a plurality of RNAs/DNAs (including the target RNA/DNA and a plurality of non-target RNAs/DNAs), wherein the target RNA/DNA is present at one or more copies per 10, 20, 25, 50, 100, 500, 10³, 5×10³, 10⁴, 5×10⁴, 10⁵, 5×10⁵, 10⁶, or 10⁷, non-target DNAs/RNAs. As used herein, the terms “RNA/DNA” and “RNAs/DNAs” shall be given their ordinary meaning, and shall also refer to DNA, or RNA, or a combination of DNA and RNA.

The threshold of detection, for a method of detecting a target RNA/DNA in a sample, can be, for example 10 nM or less. The term “threshold of detection” shall be given its ordinary meaning, and shall also describe the minimal amount of target RNA/DNA that must be present in a sample in order for detection to occur. As an illustrative example, when a threshold of detection is 10 nM, then a signal can be detected when a target RNA/DNA is present in the sample at a concentration of 10 nM or more. In some embodiments, a disclosed method has a threshold of detection of 5 nM or less, 1 nM or less, 0.5 nM or less, 0.1 nM or less, 0.05 nM or less, 0.01 nM or less, 0.005 nM or less, 0.001 nM or less, 0.0005 nM or less, 0.0001 nM or less, 0.00005 nM or less, 0.00001 nM or less, 10 pM or less, 1 pM or less, 500 fM or less, 250 fM or less, 100 fM or less, 50 fM or less, 500 aM (attomolar) or less, 250 aM or less, 100 aM or less, 50 aM or less, 10 aM or less, or 1 aM or less. In some embodiments, a disclosed composition or method exhibits an attamolar (aM), femtomolar (fM), picomolar (pM), and/or nanomolar (nM), sensitivity of detection.

A sample can comprise sample nucleic acids (e.g., a plurality of sample nucleic acids). The term “plurality” is used herein to mean two or more. Thus, in some embodiments, a sample includes two or more (e.g., 3 or more, 5 or more, 10 or more, 20 or more, 50 or more, 100 or more, 500 or more, 1,000 or more, or 5,000 or more) sample nucleic acids (e.g., DNAs/RNAs). A disclosed method can be used as a very sensitive way to detect a target nucleic acid present in a sample (e.g., in a complex mixture of nucleic acids such as DNAs/RNAs). In some embodiments the sample includes 5, 10, 20, 25, 50, 100, 500, 10³, 5×10³, 10⁴, 5×10⁴, 10⁵, 5×10⁵, 10⁶, or 10⁷, 50, or more, DNAs/RNAs that differ from one another in sequence. In some embodiments, the sample includes DNAs/RNAs from a cell (e.g., a eukaryotic cell, a mammalian cell, or a human cell) or a cell lysate (e.g., a eukaryotic cell lysate, a mammalian cell lysate, a human cell lysate, a prokaryotic cell lysate, a plant cell lysate, or the like).

The term “sample” is used here shall be given its ordinary meaning and shall include any sample that includes RNA and/or DNA (e.g., in order to determine whether a target DNA and/or target RNA is present among a population of RNAs and/or DNAs). The sample can be a biological sample or an environmental sample. The sample can be derived from any source, e.g., the sample can be a synthetic combination of purified DNAs and/or RNAs; the sample can be a cell lysate, an DNA/RNA-enriched cell lysate, or DNAs/RNAs isolated and/or purified from a cell lysate. The sample can be from a patient (e.g., for the purpose of diagnosis). The sample can be from permeabilized cells, crosslinked cells, tissue sections, or combination thereof. The sample can be from tissues prepared by crosslinking followed by delipidation and adjustment to make a uniform refractive index. A sample can include a target nucleic acid (e.g., target DNA/RNA) and a plurality of non-target DNAs/RNAs. In some embodiments, the target DNA/RNA is present in the sample at one copy per 10, 20, 25, 50, 100, 500, 10³, 5×10³, 10⁴, 5×10⁴, 10⁵, 5×10⁵, 10⁶, or 10⁷, non-target DNAs/RNAs.

A sample with respect to a patient encompasses blood and other liquid samples of biological origin, solid tissue samples such as a biopsy specimen or tissue cultures or cells derived therefrom and the progeny thereof, as well as samples that have been manipulated in any way after their procurement (such as by treatment with reagents); washed; or enriched for certain cell populations (e.g., cancer cells) or particular types of molecules (e.g., RNAs). A sample can comprise, or be, a biological sample including but not limited to a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A biological sample can comprise biological fluids derived therefrom (e.g., cancerous cell, infected cell, etc.), e.g., a sample comprising RNAs that is obtained from such cells (e.g., a cell lysate or other cell extract comprising RNAs). In some embodiments, the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof.

The source of the sample can be a (or is suspected of being a) diseased cell, fluid, tissue, or organ; or a normal (non-diseased) cell, fluid, tissue, or organ. In some embodiments, the source of the sample is a (or is suspected of being a) pathogen-infected cell, tissue, or organ. For example, the source of a sample can be an individual who may or may not be infected—and the sample can be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual, as well as cultures thereof. The sample can be a cell-free liquid sample or a liquid sample that comprise cells. Pathogens can be viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis C virus; Hepatitis A virus; Hepatitis B virus; papillomavirus; and the like. Pathogenic viruses can include DNA viruses such as: a papovavirus (e.g., HPV, polyomavirus); a hepadnavirus; a herpesvirus (e.g., HSV (e.g., HSV I, HSV II), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Non-limiting examples of pathogens include Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, human serum parvo-like virus, respiratory syncytial virus, measles virus, adenovirus, human T-cell leukemia viruses, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria sp. (e.g., tenella), Onchocerca volvulus, Leishmania sp., (e.g., tropica), Streptococcus pneumonia, Pneumocystis carinii, Trichophyton rubrum, Entamoeba histolytica, Babesia microti, Giardia lamblia, Cyclospora sp., SARS-CoV-2, Human Immunodeficiency Virus Type 1 (HIV-1), Human T-Cell Lymphotrophic Virus Type 1 (HTLV-1), Herpes Simplex, Herpesvirus 6, Herpesvirus 7, JC Virus, Influenza A, Influenza B, Influenza C, Rotavirus, Human Adenovirus, Human Enteroviruses, Hantavirus, Legionella dumoffii, Mycoplasma fermentans, Haemophilus influenzae, Rickettsia rickettsii, Ehrlichia sp. (e.g., chaffeensis), Borrelia burgdorferi, Yersinia pestis, Chlamydia pneumoniae, Trichinella spiralis, Theileria parva, Taenia hydatigena, Taenia ovis, Taenia saginata, Echinococcus granulosus, Mesocestoides corti, Mycoplasma sp. (e.g, arthritidis), M. hyorhinis, M. orale, M. arginini, Acholeplasma laidlawii, M. salivarium and M. pneumoniae.

### Amplification

Provided herein are methods for amplifying nucleic acid. In some embodiments, nucleic acids are amplified using a suitable amplification process. Nucleic acid amplification typically involves enzymatic synthesis of nucleic acid amplicons (copies), which contain a sequence complementary to a nucleotide sequence being amplified. In some embodiments, an amplification method is performed in a single vessel, a single chamber, and/or a single volume (i.e., contiguous volume). In some embodiments, an amplification method and a detection method (e.g., a detection method described herein) are performed in a single vessel, a single chamber, and/or a single volume (i.e., contiguous volume).

The terms “amplify”, “amplification”, “amplification reaction”, or “amplifying” refer to any in vitro process for multiplying the copies of a target nucleic acid. Amplification sometimes refers to an “exponential” increase in target nucleic acid. “Amplifying” can also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

A generalized description of an amplification process is presented herein. Primers (e.g., oligonucleotides described herein) and target nucleic acid are contacted, and complementary sequences anneal or hybridize to one another, for example. Primers can anneal to a target nucleic acid, at or near (e.g., adjacent to, abutting, and the like) a sequence of interest. A primer annealed to a target may be referred to as a primer-target hybrid, hybridized primer-target, or a primer-target duplex. The terms near or adjacent to when referring to a nucleotide sequence of interest refer to a distance (e.g., number of bases) or region between the end of the primer and the nucleotide or nucleotides (e.g., nucleotide sequence) of a target. Generally, adjacent is in the range of about 1 nucleotide to about 50 nucleotides (e.g., about 1, 2, 3, 4, 5, 6, 7, 8, 9, 10, 20, 30, 40, or 50 nucleotide(s)) away from a nucleotide or nucleotide sequence of interest. In some embodiments, primers in a set (e.g., a pair of primers, a forward and a reverse primer, a first oligonucleotide and a second oligonucleotide) anneal within about 1 to 20 nucleotides from a nucleotide or nucleotide sequence of interest and produce amplified products. In some embodiments, primers anneal within a nucleotide or a nucleotide sequence of interest. After annealing, each primer is extended along the target (i.e., template strand) by a polymerase to generate a complementary strand. Several cycles of primer annealing and extension can be carried out, for example, until a detectable amount of amplification product is generated. In some embodiments, where a target nucleic acid is RNA, a DNA copy (cDNA) of the target RNA is synthesized prior to or during the amplification step by reverse transcription.

Components of an amplification reaction (e.g., the one or more amplification reagents) can include, for example, one or more primers (e.g., individual primers, primer pairs, primer sets, oligonucleotides, multiple primer sets for multiplex amplification, and the like), nucleic acid target(s) (e.g., target nucleic acid from a sample), one or more polymerases, nucleotides (e.g., dNTPs and the like), and a suitable buffer (e.g., a buffer comprising a detergent, a reducing agent, monovalent ions, and divalent ions). An amplification reaction can further include one or more of: a reverse transcriptase, a reverse transcription primer, and one or more detection agents.

Nucleic acid amplification can be conducted in the presence of native nucleotides, for example, dideoxyribonucleoside triphosphates (dNTPs), and/or derivatized nucleotides. A native nucleotide generally refers to adenylic acid, guanylic acid, cytidylic acid, thymidylic acid, or uridylic acid. A derivatized nucleotide generally is a nucleotide other than a native nucleotide. A ribonucleoside triphosphate is referred to as NTP or rNTP, where N can be A, G, C, U. A deoxynucleoside triphosphate substrates is referred to as dNTP, where N can be A, G, C, T, or U. Monomeric nucleotide subunits may be denoted as A, G, C, T, or U herein with no particular reference to DNA or RNA. In some embodiments, non-naturally occurring nucleotides or nucleotide analogs, such as analogs containing a detectable label (e.g., fluorescent or colorimetric label), may be used. For example, nucleic acid amplification can be carried out in the presence of labeled dNTPs, for example, radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, or fluorochromes. In some embodiments, nucleic acid amplification may be carried out in the presence of modified dNTPs, for example, heat activated dNTPs (e.g., CleanAmp™ dNTPs from TriLink).

The one or more amplification reagents can include non-enzymatic components and enzymatic components. Non-enzymatic components can include, for example, primers, nucleotides, buffers, salts, reducing agents, detergents, and ions. In some embodiments, the Non-enzymatic components do not include proteins (e.g., nucleic acid binding proteins), enzymes, or proteins having enzymatic activity, for example, polymerases, reverse transcriptases, helicases, topoisomerases, ligases, exonucleases, endonucleases, restriction enzymes, nicking enzymes, recombinases and the like. In some embodiments, an enzymatic component consists of a polymerase or consists of a polymerase and a reverse transcriptase. Accordingly, such enzymatic components would exclude other proteins (e.g., nucleic acid binding proteins and/or proteins having enzymatic activity), for example, helicases, topoisomerases, ligases, exonucleases, endonucleases, restriction enzymes, nicking enzymes, recombinases, and the like.

In some embodiments, amplification conditions comprise an enzymatic activity (e.g., an enzymatic activity provided by a polymerase or provided by a polymerase and a reverse transcriptase). In some embodiments, the enzymatic activity does not include enzymatic activity provided by enzymes other than the polymerase and/or the reverse transcriptase, for example, helicases, topoisomerases, ligases, exonucleases, endonucleases, restriction enzymes, nicking enzymes, recombinases, and the like. A polymerase activity and a reverse transcriptase activity can be provided by separate enzymes or separate enzyme types (e.g., polymerase(s) and reverse transcriptase(s)), or provided by a single enzyme or enzyme type (e.g., polymerase(s)).

Amplification of nucleic acid can comprise a non-thermocycling type of PCR. In some embodiments, amplification of nucleic acid comprises an isothermal amplification process, for example an isothermal polymerase chain reaction (iPCR). Isothermal amplification generally is an amplification process performed at a constant temperature. Terms such as isothermal conditions, isothermally and constant temperature generally refer to reaction conditions where the temperature of the reaction is kept essentially constant during the course of the amplification reaction. Isothermal amplification conditions generally do not include a thermocycling (i.e., cycling between an upper temperature and a lower temperature) component in the amplification process. When amplifying under isothermal conditions, the reaction can be kept at an essentially constant temperature, which means the temperature may not be maintained at precisely one temperature. For example, small fluctuations in temperature (e.g., ±1 to 5 °C) may occur in an isothermal amplification process due to, for example, environmental or equipment-based variables. Often, the entire reaction volume is kept at an essentially constant temperature, and isothermal reactions herein generally do not include amplification conditions that rely on a temperature gradient generated within a reaction vessel and/or convective-flow based temperature cycling.

Isothermal amplification reactions herein can be conducted at an essentially constant temperature. In some embodiments, isothermal amplification reactions herein are conducted at a temperature of about 55 °C to a temperature of about 75 °C, for example at a temperature of, or a temperature of about, 55, 56, 57, 58, 59, 60, 61, 62, 63, 64, 65, 66, 67, 68, 69, 70, 71, 72, 73, 74, or about 75 °C, or a number or a range between any two of these values. In some embodiments, a temperature element (e.g., heat source) is kept at an essentially constant temperature, for example an essentially constant temperature at or below about 75 °C, at or below about 70 degrees Celsius, at or below about 65 °C, or at or below about 60 °C.

An amplification process herein can be conducted over a certain length of time, for example until a detectable nucleic acid amplification product is generated. A nucleic acid amplification product may be detected by any suitable detection process and/or a detection process described herein. The amplification process can be conducted over a length of time within about 20 minutes or less, or about 10 minutes or less. For example, an amplification process can be conducted within about 1, 2, 3, 4, 5, 6, 7, 8, 9, 10, 11, 12, 13, 14, 15, 16, 17, 18, 19 or 20 minutes, or a number or a range between any two of these values.

Nucleic acid targets can be amplified without exposure to agents or conditions that denature nucleic acid, in some embodiments. Nucleic acid targets can be amplified without exposure to agents or conditions that promote strand separation during the amplification step (and/or other steps) in some embodiments. Nucleic acid targets can be amplified without exposure to agents or conditions that promote unwinding during the amplification step (and/or other steps) in some embodiments. Agents or conditions that denature nucleic acid and/or promote strand separation and/or promote unwinding may include, for example, thermal conditions (e.g., high temperatures), pH conditions (e.g., high or low pH), chemical agents, proteins (e.g., enzymatic agents), and the like.

In some embodiments, the methods disclosed herein does not comprise thermal denaturation (e.g., heating a solution containing a nucleic acid to an elevated temperature, such as, for example a temperature above 75 °C, 80 °C, 90 °C, or 95 °C, or higher) or protein-based (e.g., enzymatic) denaturation of a nucleic acid. Protein-based (e.g., enzymatic) denaturation can comprise contacting a nucleic acid with one or more of a helicase, a topoisomerase, a ligase, an exonuclease, an endonuclease, a restriction enzyme, a nicking enzyme, a recombinase, a RNA replicase, and a nucleic acid binding protein (e.g., single-stranded binding protein). In some embodiments, the compositions provided herein do not comprise a helicase, a topoisomerase, a ligase, an exonuclease, an endonuclease, a restriction enzyme, a nicking enzyme, a recombinase, a RNA replicase, and/or a nucleic acid binding protein (e.g., single-stranded binding protein). In some embodiments, the compositions and methods provided herein do not comprise intercalators, alkylating agents, and/or chemicals such as formamide, glycerol, urea, dimethyl sulfoxide (DMSO), or N,N,N-trimethylglycine (betaine). In some embodiments, the disclosed methods do not comprise contacting a nucleic acid with denaturing agents (e.g., formamide). In some embodiments, the amplifying step does not comprise agents and/or conditions that denature nucleic acids (e.g., promote strand separation and/or promote unwinding). In some embodiments, the amplifying step (e.g., step (c)) does not comprise agents and/or conditions that denature nucleic acids (e.g., promote strand separation and/or promote unwinding) other than a polymerase (e.g., a hyperthermophile polymerase). In some embodiments, the methods and compositions provided herein not comprise agents and/or conditions that denature nucleic acids (e.g., promote strand separation and/or promote unwinding) other than a polymerase (e.g., a hyperthermophile polymerase) and/or low pH conditions (e.g., contact with acid(s)).

Nucleic acid targets can be amplified without exposure to agents or conditions that promote strand separation and/or unwinding, for example a helicase, a topoisomerase, a ligase, an exonuclease, an endonuclease, a restriction enzyme, a nicking enzyme, a recombinase, a RNA replicase, a nucleic acid binding protein (e.g., single-stranded binding protein), or any combination thereof. For example, nucleic acid targets can be amplified without exposure to a helicase, including but not limited to DNA helicases and RNA helicases. Amplification conditions that do not include use of a helicase are helicase-free amplification conditions.

Nucleic acid targets can be amplified without exposure to a recombinase, including but not limited to, Cre recombinase, Hin recombinase, Tre recombinase, FLP recombinase, RecA, RAD51, RadA, T4 uvsX. In some embodiments, nucleic acid targets are amplified without exposure to a recombinase accessory protein, for example, a recombinase loading factor (e.g., T4 uvsY). Nucleic acid targets can be amplified without exposure to a nucleic acid binding protein (e.g., single-stranded binding protein or single-strand DNA-binding protein (SSB)), for example, T4 gp32. In some embodiments, nucleic acid targets are amplified without exposure to a topoisomerase. Nucleic acid targets can be amplified with or without exposure to agents or conditions that destabilize nucleic acid. As used herein, the term “destabilization” shall be given its ordinary meaning, and shall also refer to a disruption in the overall organization and geometric orientation of a nucleic acid molecule (e.g., double helical structure) by one or more of tilt, roll, twist, slip, and flip effects (e.g., as described in Lenglet et al., (2010) Journal of Nucleic Acids Volume 2010, Article ID 290935, 17 pages). Destabilization generally does not refer to melting or separation of nucleic acid strands (e.g., denaturation). Nucleic acid destabilization can be achieved, for example, by exposure to agents such as intercalators or alkylating agents, and/or chemicals such as formamide, urea, dimethyl sulfoxide (DMSO), or N,N,N-trimethylglycine (betaine). In some embodiments, methods provided herein include use of one or more destabilizing agents. In some embodiments, methods provided herein exclude use of destabilizing agents. In some embodiments, nucleic acid targets are amplified without exposure to a ligase and/or an RNA replicase.

Nucleic acid targets can be amplified without cleavage or digestion, in some embodiments. For example, nucleic acid targets can be amplified without prior exposure to one or more cleavage agents, and intact nucleic acid is amplified. In some embodiments, nucleic acid targets are amplified without exposure to one or more cleavage agents during amplification. In some embodiments, nucleic acid targets are amplified without exposure to one or more cleavage agents after amplification. Amplification conditions that do not include use of a cleavage agent may be referred to herein as cleavage agent-free amplification conditions. The term “cleavage agent” generally refers to an agent, sometimes a chemical or an enzyme that can cleave a nucleic acid at one or more specific or non-specific sites. Specific cleavage agents often cleave specifically according to a particular nucleotide sequence at a particular site. Cleavage agents can include endonucleases (e.g., restriction enzymes, nicking enzymes, and the like); exonucleases (DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases); and chemical cleavage agents.

Nucleic acid targets can be amplified without use of restriction enzymes and/or nicking enzymes. In some embodiments, nucleic acid is amplified without prior exposure to restriction enzymes and/or nicking enzymes. In some embodiments, nucleic acid is amplified without exposure to restriction enzymes and/or nicking enzymes during amplification. In some embodiments, nucleic acid is amplified without exposure to restriction enzymes and/or nicking enzymes after amplification. Nucleic acid targets can be amplified without exonuclease treatment. Exonucleases include, for example, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In some embodiments, nucleic acid is amplified without exonuclease treatment prior to, during, and/or after amplification. Amplification conditions that do not include use of an exonuclease are exonuclease-free amplification conditions. In some embodiments, nucleic acid is amplified without DNAse treatment and/or RNAse treatment. In some embodiments, nucleic acid is amplified without RNAseH treatment.

An amplified nucleic acid may be referred to herein as a nucleic acid amplification product or amplicon. In some embodiments, the amplification product includes naturally occurring nucleotides, non-naturally occurring nucleotides, nucleotide analogs and the like and combinations of the foregoing. An amplification product typically has a nucleotide sequence that is identical to or substantially identical to a sequence in a sample nucleic acid (e.g., target sequence) or complement thereof. A “substantially identical” nucleotide sequence in an amplification product will generally have a high degree of sequence identity to the nucleotide sequence being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of polymerase infidelity or other variables.

A nucleic acid amplification product can comprise a polynucleotide that is continuously complementary to or substantially identical to a target sequence in sample nucleic acid. Continuously complementary generally refers to a nucleotide sequence in a first strand, for example, where each base in order (e.g., read 5′ to 3′) pairs with a correspondingly ordered base in a second strand, and there are no gaps, additional sequences or unpaired bases within the sequence considered as continuously complementary. Stated another way, continuously complementary generally refers to all contiguous bases of a nucleotide sequence in a first stand being complementary to corresponding contiguous bases of a nucleotide sequence in a second strand. For example, a first strand having a sequence 5′-ATGCATGCATGC-3′ (SEQ ID NO: 9) would be considered as continuously complementary to a second strand having a sequence 5′-GCATGCATGCAT-3′ (SEQ ID NO: 10), where all contiguous bases in the first strand are complementary to all corresponding contiguous bases in the second strand. However, a first strand having a sequence 5′-ATGCATAAAAAAGCATGC-3′ (SEQ ID NO: 11) would not be considered as continuously complementary to a second strand having a sequence 5′-GCATGCATGCAT-3′ (SEQ ID NO: 10), because the sequence of six adenines (6 As) in the middle of the first strand would not pair with bases in the second strand. A continuously complementary sequence sometimes is about 5 to about 25 contiguous bases in length, e.g., about 5, 6, 7, 8, 9, 10, 11, 12, 13, 14, 15, 16, 17, 18, 19, 20, 21, 22, 23, 24, 25, or a range between any two of these values, contiguous bases in length. In some embodiments, a nucleic acid amplification product consists of a polynucleotide that is continuously complementary to or substantially identical to a target sequence in sample nucleic acid. Accordingly, in some embodiments, a nucleic acid amplification product does not include any additional sequences (e.g., at the 5′ and/or 3′ end, or within the product) that are not continuously complementary to or substantially identical to a target sequence, for example, additional sequences incorporated into an amplification product by way of tailed primers or ligation, and/or additional sequences providing cleavage agent recognition sites (e.g., nicking enzyme recognition sites). Generally, unless a target sequence comprises tandem repeats, an amplification product does not include product in the form of tandem repeats.

Nucleic acid amplification products can comprise sequences complementary to or substantially identical to one or more primers used in an amplification reaction. In some embodiments, a nucleic acid amplification product comprises a first nucleotide sequence that is continuously complementary to or identical to a first primer sequence, and a second nucleotide sequence that is continuously complementary to or identical to a second primer sequence.

Nucleic acid amplification products can comprise a spacer sequence. As described herein, a spacer sequence in an amplification product is a sequence (1 or more bases) continuously complementary to or substantially identical to a portion of a target sequence in the sample nucleic acid, and is flanked by sequences in the amplification product that are complementary to or substantially identical to one or more primers used in an amplification reaction. A spacer sequence flanked by sequences in the amplification product generally lies between a first sequence (complementary to or substantially identical to a first primer) and a second sequence (complementary to or substantially identical to a second primer). Thus, an amplification product typically includes a first sequence followed by a spacer sequences followed by a second sequence. A spacer sequence generally is not complementary to or substantially identical to a sequence in the primer(s). A spacer sequence can be, or can comprise, about 1 to 10 bases, including 1, 2, 3, 4, 5, 6, 7, 8, 9, or 10 bases. In some embodiments, a nucleic acid amplification product consists of, or consists essentially of, a first nucleotide sequence that is continuously complementary to or identical to a first primer sequence, a second nucleotide sequence that is continuously complementary to or identical to a second primer sequence, and a spacer sequence. In some embodiments, a nucleic acid amplification product does not include any additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. In some embodiments, a nucleic acid amplification product generally does not include additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. However, in such embodiments, a nucleic acid amplification product may include, for example, some mismatched (i.e., non-complementary) bases or one more extra bases (e.g., at the 5′ and/or 3′ end; or within the product) introduced into the product by way of error or promiscuity in the amplification process.

Nucleic acid amplification products can be up to 50 bases in length, including 10, 15, 20, 25, 30, 35, 40, 45, 50, or a number or a range between any two of these values, bases long. In some embodiments, nucleic acid amplification products for a given target sequence have the same length or substantially the same length (e.g., within 1 to 10 bases). Accordingly, nucleic acid amplification products for a given target sequence may produce a single signal (e.g., band on an electrophoresis gel) and generally do not produce multiple signals indicative of multiple lengths (e.g., a ladder or smear on an electrophoresis gel). For multiplex reactions, nucleic acid amplification products for different target sequences may have different lengths.

The methods and components described herein can be used for multiplex amplification which generally refers to the amplification of more than one nucleic acid of interest (e.g., amplification or more than one target sequence). For example, multiplex amplification can refer to amplification of multiple sequences from the same sample or amplification of one of several sequences in a sample. For example, the amplifying step can comprise multiplex amplification of two or more target nucleic acid sequences and the detecting step can comprise multiplex detection of two or more nucleic acid amplification products derived from said two or more target nucleic acid sequences. The two or more target nucleic acid sequences can specific to two or more different organisms (e.g., one or more of SARS-CoV-2, Influenza A, Influenza B, and/or Influenza C). Multiplex amplification also can refer to amplification of one or more sequences present in multiple samples either simultaneously or in step-wise fashion. For example, a multiplex amplification can be used for amplifying least two target sequences that are capable of being amplified (e.g., the amplification reaction comprises the appropriate primers and enzymes to amplify at least two target sequences). In some embodiments, an amplification reaction is prepared to detect at least two target sequences, but only one of the target sequences is present in the sample being tested, such that both sequences are capable of being amplified, but only one sequence is amplified. In some embodiments, where two target sequences are present, an amplification reaction results in the amplification of both target sequences. A multiplex amplification reaction can result in the amplification of one, some, or all of the target sequences for which it comprises the appropriate primers and enzymes. In some embodiments, an amplification reaction is prepared to detect two sequences with one pair of primers, where one sequence is a target sequence and one sequence is a control sequence (e.g., a synthetic sequence capable of being amplified by the same primers as the target sequence and having a different spacer base or sequence than the target). In some embodiments, an amplification reaction is prepared to detect multiple sets of sequences with corresponding primer pairs, where each set includes a target sequence and a control sequence.

### Primers

Nucleic acid amplification generally is conducted in the presence of one or more primers. A primer is generally characterized as an oligonucleotide that includes a nucleotide sequence capable of hybridizing or annealing to a target nucleic acid, at or near (e.g., adjacent to) a specific region of interest (i.e., target sequence). Primers can allow for specific determination of a target nucleic acid nucleotide sequence or detection of the target nucleic acid (e.g., presence or absence of a sequence), or feature thereof, for example. A primer can be naturally occurring or synthetic. The term specific, or specificity, generally refers to the binding or hybridization of one molecule to another molecule, such as a primer for a target polynucleotide. That is, specific or specificity refers to the recognition, contact, and formation of a stable complex between two molecules, as compared to substantially less recognition, contact, or complex formation of either of those two molecules with other molecules. The term anneal or hybridize generally refers to the formation of a stable complex between two molecules. The terms primer, oligo, or oligonucleotide may be used interchangeably herein, when referring to primers.

A primer can be designed and synthesized using suitable processes, and can be of any length suitable for hybridizing to a target sequence and performing an amplification process described herein. Primers often are designed according to a sequence in a target nucleic acid. A primer in some embodiments may be about 5 to about 30 bases in length, for example 5, 6, 7, 8, 9, 10, 11, 12, 13, 14, 15, 16, 17, 18, 19, 20, 21, 22, 23, 24, 25, 26, 27, 28, 29 or 30 bases in length. A primer may be composed of naturally occurring and/or non-naturally occurring nucleotides (e.g., modified nucleotides, labeled nucleotides), or a mixture thereof. Modifications and modified bases may include, for example, phosphorylation, (e.g., 3′ phosphorylation, 5′ phosphorylation); attachment chemistry or linkers modifications (e.g., Acrydite™, adenylation, azide (NHS ester), digoxigenin (NHS ester), cholesteryl-TEG, I-Linker™, amino modifiers (e.g., amino modifier C6, amino modifier C12, amino modifier C6 dT, Uni-Link™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S-S, dithiol, thiol modifier C6 S-S)); fluorophores (e.g., Freedom™ Dyes, Alexa Fluor® Dyes, LI-COR IRDyes®, ATTO™ Dyes, Rhodamine Dyes, WellRED Dyes, 6-FAM (azide), Texas Red®-X (NHS ester), Lightcycler® 640 (NHS ester), Dy 750 (NHS ester)); Iowa Black® dark quenchers modifications (e.g., Iowa Black® FQ, Iowa Black® RQ); dark quenchers modifications (e.g., Black Hole Quencher®-1, Black Hole Quencher®-2, Dabcyl); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxyInosine, Super T®, Super G®, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate (PS) bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, PS linkages, 3′ phosphate groups, spacer bases (such as C3 spacer or other spacer bases). For example, a primer may comprise one or more O-methyl RNA bases (e.g., 2′-O-methyl RNA bases). 2′-O-methyl RNA generally is a post-transcriptional modification of RNA found in tRNA and other small RNAs. Primers can be directly synthesized that include 2′-O-methyl RNA bases. This modification can, for example, increase Tm of RNA:RNA duplexes and provide stability in the presence of single-stranded ribonucleases and DNases. 2′-O-methyl RNA bases may be included in primers, for example, to increase stability and binding affinity to a target sequence. In some embodiments, a primer may comprise one or more phosphorothioate (PS) linkages (e.g., PS bond modifications). A PS bond substitutes a sulfur atom for a non-bridging oxygen in the phosphate backbone of a primer. This modification typically renders the internucleotide linkage resistant to nuclease degradation. PS bonds can be introduced between about the last 3 to 5 nucleotides at the 5′-end or the 3′-end of a primer to inhibit exonuclease degradation, for example. PS bonds included throughout an entire primer can help reduce attack by endonucleases, in some embodiments. A primer can, for example, comprise a 3′ phosphate group. 3′ phosphorylation can inhibit degradation by certain 3′-exonucleases and can be used to block extension by DNA polymerases, in certain instances. In some embodiments, a primer comprises one or more spacer bases (e.g., one or more C3 spacers). A C3 spacer phosphoramidite can be incorporated internally or at the 5′-end of a primer. Multiple C3 spacers can be added at either end of a primer to introduce a long hydrophilic spacer arm for the attachment of fluorophores or other pendent groups, for example.

A primer can comprises DNA bases, RNA bases, or both, where one or more of the DNA bases and RNA bases is modified or unmodified. For example, a primer can be a mixture of DNA bases and RNA bases. The primer can consist of DNA bases (e.g., modified DNA bases and/or unmodified DNA bases). In some embodiments, the primer consists of unmodified DNA bases. In some embodiments, the primer consists of modified DNA bases. The primer can consist of RNA bases (e.g., modified RNA bases and/or unmodified RNA bases). In some embodiments, the primer consists of unmodified RNA bases. In some embodiments, the primer consists of modified RNA bases. In some embodiments, a primer comprises no RNA bases. In some embodiments, a primer comprises no DNA bases. In some embodiments, the primer comprises no cleavage agent recognition sites (e.g., no nicking enzyme recognition sites). In some embodiments, a primer comprises no tail (e.g., no tail comprising a nicking enzyme recognition site).

All or a portion of a primer sequence can be complementary or substantially complementary to a target nucleic acid, in some embodiments. Substantially complementary with respect to sequences generally refers to nucleotide sequences that will hybridize with each other. The stringency of the hybridization conditions can be altered to tolerate varying amounts of sequence mismatch. The target and primer sequences can be, for example, at least 75% complementary to each other, including 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to each other. Primers that are substantially complimentary to a target nucleic acid sequence typically are also substantially identical to the complement of the target nucleic acid sequence (i.e., the sequence of the anti-sense strand of the target nucleic acid). The primer and the anti-sense strand of the target nucleic acid can be at least 75% identical in sequence, for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each other.

In some embodiments, primers comprise a pair of primers. A pair of primers may include a forward primer and a reverse primer (e.g., primers that bind to the sense and antisense strands of a target nucleic acid). In some embodiments, primers consist of a pair of primers (i.e. a forward primer and a reverse primer). Accordingly, in some embodiments, amplification of a target sequence is performed using a pair of primers and no additional primers or oligonucleotides are included in the amplification of the target sequence (e.g., the amplification reaction components comprise no additional primer pairs for a given target sequence, no nested primers, no bumper primers, no oligonucleotides other than the primers, no probes, and the like). In some embodiments, primers consist of a pair of primers. In some embodiments, an amplification reaction can include additional primer pairs for amplifying different target sequences, such as in a multiplex amplification. In some embodiments, primers consist of a pair of primers, however, in some embodiments, an amplification reaction can include additional primers, oligonucleotides or probes for a detection process that are not considered part of amplification. In some embodiments, primers are used in sets. An amplification primer set can include a pair of forward and reverse primers for a given target sequence. For multiplex amplification, primers that amplify a first target sequence are considered a primer set, and primers that amplify a second target sequence are considered a different primer set.

Nucleic acids described herein (e.g., amplification products, sample nucleic acids, target nucleic acid sequences) can comprise a first strand and a second strand complementary to each other. Amplification reaction components can comprise, or consist of, a first primer (first oligonucleotide) complementary to a target sequence in a first strand (e.g., sense strand, forward strand) of a sample nucleic acid, and a second primer (second oligonucleotide) complementary to a target sequence in a second strand (e.g., antisense strand, reverse strand) of a sample nucleic acid. In some embodiments, a first primer (first oligonucleotide) comprises a first polynucleotide continuously complementary to a target sequence in a first strand of sample nucleic acid, and a second primer (second oligonucleotide) comprises a second polynucleotide continuously complementary to a target sequence in a second strand of sample nucleic acid. Continuously complementary for a primer-target generally refers to a nucleotide sequence in a primer, where each base in order pairs with a correspondingly ordered base in a target sequence, and there are no gaps, additional sequences or unpaired bases within the sequence considered as continuously complementary. In some embodiments, a primer does not include any additional sequences (e.g., at the 5′ and/or 3′ end, or within the primer) that are not continuously complementary to a target sequence, for example, additional sequences present in tailed primers or looped primers, and/or additional sequences providing cleavage agent recognition sites (e.g., nicking enzyme recognition sites). In some embodiments, amplification reaction components do not comprise primers comprising additional sequences (i.e., sequences other than the sequence that is continuously complementary to a target sequence), for example, tailed primers, looped primers, primers capable of forming step-loop structures, hairpin structures, and/or additional sequences providing cleavage agent recognition sites (e.g., nicking enzyme recognition sites), and the like.

The primer, in some embodiments, can contain a modification such as one or more inosines, abasic sites, locked nucleic acids, minor groove binders, duplex stabilizers (e.g., acridine, spermidine), Tm modifiers or any modifier that changes the binding properties of the primer. The primer, in some embodiments, can contain a detectable molecule or entity (e.g., a fluorophore, radioisotope, colorimetric agent, particle, enzyme and the like).

### Polymerase

Amplification reaction components (e.g., one or more amplification reagents) can comprise one or more polymerases. Polymerases are proteins capable of catalyzing the specific incorporation of nucleotides to extend a 3′ hydroxyl terminus of a primer molecule, for example, an amplification primer described herein, against a nucleic acid target sequence (e.g., to which a primer is annealed). Non-limiting examples of polymerases include thermophilic or hyperthermophilic polymerases that can have activity at an elevated reaction temperature (e.g., above 55, 60, 65, 70, 75, 80, 85, 90, 95, or 100 °C). A hyperthermophilic polymerase may be referred to as a hyperthermophile polymerase. A polymerase may or may not have strand displacement capabilities. In some embodiments, a polymerase can incorporate about 1 to about 50 nucleotides in a single synthesis, for example about 1, 5, 10, 15, 20, 25, 30, 35, 40, 45 or 50 nucleotides, or a number or a range between any two of these values, in a single synthesis.

The amplification reaction components can comprise one or more DNA polymerases selected from: 9° N DNA polymerase; 9° Nm™ DNA polymerase; Therminator™ DNA Polymerase; Therminator™ II DNA Polymerase; Therminator™ III DNA Polymerase; Therminator™ γ DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; Deep VentR™ (exo-) DNA Polymerase; Deep VentR™ DNA Polymerase; DyNAzyme™ EXT DNA; DyNAzyme™ II Hot Start DNA Polymerase; Phusion™ High-Fidelity DNA Polymerase; VentR® DNA Polymerase; VentR® (exo-) DNA Polymerase; RepliPHI™ Phi29 DNA Polymerase; rBst DNA Polymerase, large fragment (IsoTherm™ DNA Polymerase); MasterAmp™ AmpliTherm™ DNA Polymerase; Tag DNA polymerase; Tth DNA polymerase; Tfl DNA polymerase; Tgo DNA polymerase; SP6 DNA polymerase; Tbr DNA polymerase; DNA polymerase Beta; and ThermoPhi DNA polymerase.

In some embodiments, the amplification reaction components comprise one or more hyperthermophile DNA polymerases (e.g., hyperthermophile DNA polymerases that are thermostable at high temperatures). The hyperthermophile DNA polymerase can have a half-life of about 5 to 10 hours at 95 °C and a half-life of about 1 to 3 hours at 100 °C. For example, the amplification reaction components can comprise one or more hyperthermophile DNA polymerases from Archaea (e.g., hyperthermophile DNA polymerases from Thermococcus, or hyperthermophile DNA polymerases from Thermococcaceaen archaean). In some embodiments, amplification reaction components comprise one or more hyperthermophile DNA polymerases from Pyrococcus, Methanococcaceae, Methanococcus, or Thermus. In some embodiments, amplification reaction components comprise one or more hyperthermophile DNA polymerases from Thermus thermophiles.

In some embodiments, amplification reaction components comprise a hyperthermophile DNA polymerase or functional fragment thereof. A functional fragment generally retains one or more functions of a full-length polymerase, for example, the capability to polymerize DNA (e.g., in an amplification reaction). In some instances, a functional fragment performs a function (e.g., polymerization of DNA in an amplification reaction) at a level that is at least about 50%, at least about 75%, at least about 90%, at least about 95% the level of function for a full length polymerase. Levels of polymerase activity can be assessed, for example, using a detectable nucleic acid amplification method, such as a method described herein. In some embodiments, amplification reaction components comprise a hyperthermophile DNA polymerase comprising an amino acid sequence of SEQ ID NO: 7 or SEQ ID NO: 8, or a functional fragment of SEQ ID NO: 7 or SEQ ID NO: 8.

In some embodiments, amplification reaction components (e.g., one or more amplification reagents) comprise a polymerase comprising an amino acid sequence that is at least about 90% identical to a hyperthermophile polymerase or a functional fragment thereof. In some embodiments, amplification reaction components comprise a polymerase comprising an amino acid sequence that is at least about 90%, 95%, or 99% identical to the amino acid sequence of SEQ ID NO: 7 or SEQ ID NO: 8, or a functional fragment thereof.

The polymerase can possess reverse transcription capabilities. In such embodiments, the amplification reaction can amplify RNA targets, for example, in a single step without the use of a separate reverse transcriptase. Non-limiting examples of polymerases that possess reverse transcriptase capabilities include Bst (large fragment), 9° N DNA polymerase, 9° Nm™ DNA polymerase, Therminator™, Therminator™ II, and the like). Amplification reaction components can comprise one or more separate reverse transcriptases. In some embodiments, more than one polymerase is included in in an amplification reaction. For example, an amplification reaction may comprise a polymerase having reverse transcriptase activity and a second polymerase having no reverse transcriptase activity.

In some embodiments, one or more polymerases having exonuclease activity are used during amplification. In some embodiments, one or more polymerases having no or low exonuclease activity are used during amplification. In some embodiments, a polymerase having no or low exonuclease activity comprises one or more modifications (e.g., amino acid substitutions) that reduce or eliminate the exonuclease activity of the polymerase. For example, a modified polymerase having low exonuclease activity can have 10% or less exonuclease activity compared to an unmodified polymerase, for example less than about 10%, 9%, 8%, 7%, 6%, 5%, 4%, 3%, 2%, or 1% exonuclease activity compared to an unmodified polymerase. In some embodiments, a polymerase has no or low 5′ to 3′ exonuclease activity, and/or no or low 3′ to 5′ exonuclease activity. In some embodiments, a polymerase has no or low single strand dependent exonuclease activity, and/or no or low double strand dependent exonuclease activity. Nonlimiting examples of the modifications that can reduce or eliminate exonuclease activity for a polymerase include one or more amino acid substitutions at position 141 and/or 143 and/or 458 of SEQ ID NO: 7 (e.g., D141A, E143A, E143D and A485L), or at a position corresponding to position 141 and/or 143 and/or 458 of SEQ ID NO: 7.

### Detection and Quantification

The methods described herein can comprise detecting and/or quantifying nucleic acid amplification product(s). Amplification product(s) can be detected and/or quantified, for example, by any suitable detection and/or quantification method described herein. Non-limiting examples of detection and/or quantification methods include molecular beacon (e.g., real-time, endpoint), lateral flow, fluorescence resonance energy transfer (FRET), fluorescence polarization (FP), surface capture, 5′ to 3′ exonuclease hydrolysis probes (e.g., TAQMAN), intercalating/binding dyes, absorbance methods (e.g., colorimetric, turbidity), electrophoresis (e.g., gel electrophoresis, capillary electrophoresis), mass spectrometry, nucleic acid sequencing, digital amplification, a primer extension method (e.g., iPLEX™), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, AlphaScreen, SNPstream, genetic bit analysis (GBA), Multiplex minisequencing, SNaPshot, GOOD assay, Microarray miniseq, arrayed primer extension (APEX), Microarray primer extension, Tag arrays, Coded microspheres, Template-directed incorporation (TDI), colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), nanopore sequencing, chips and combinations thereof. In some embodiments, detecting a nucleic acid amplification product comprises use of a real-time detection method (i.e., product is detected and/or continuously monitored during an amplification process). In some embodiments, detecting a nucleic acid amplification product comprises use of an endpoint detection method (i.e., product is detected after completing or stopping an amplification process). Nucleic acid detection methods may also employ the use of labeled nucleotides incorporated directly into a target sequence or into probes containing complementary sequences to a target. Such labels may be radioactive and/or fluorescent in nature and can be resolved in any of the manners discussed herein. In some embodiments, quantification of a nucleic acid amplification product may be achieved using one or more detection methods described below. In some embodiments, the detection method can be used in conjunction with a measurement of signal intensity, and/or generation of (or reference to) a standard curve and/or look-up table for quantification of a nucleic acid amplification product.

Detecting a nucleic acid amplification product can comprise use of molecular beacon technology. The term molecular beacon generally refers to a detectable molecule, where the detectable property of the molecule is detectable under certain conditions, thereby enabling the molecule to function as a specific and informative signal. Non-limiting examples of detectable properties include optical properties (e.g., fluorescence), electrical properties, magnetic properties, chemical properties and time or speed through an opening of known size. Molecular beacons for detecting nucleic acid molecules can be, for example, hair-pin shaped oligonucleotides containing a fluorophore on one end and a quenching dye on the opposite end. The loop of the hair-pin can contain a probe sequence that is complementary to a target sequence and the stem is formed by annealing of complementary arm sequences located on either side of the probe sequence. A fluorophore and a quenching molecule can be covalently linked at opposite ends of each arm. Under conditions that prevent the oligonucleotides from hybridizing to its complementary target or when the molecular beacon is free in solution, the fluorescent and quenching molecules are proximal to one another preventing FRET. When the molecular beacon encounters a target molecule (e.g., a nucleic acid amplification product), hybridization can occur, and the loop structure is converted to a stable more rigid conformation causing separation of the fluorophore and quencher molecules leading to fluorescence. Due to the specificity of the probe, the generation of fluorescence generally is exclusively due to the synthesis of the intended amplified product. In some instances, a molecular beacon probe sequence hybridizes to a sequence in an amplification product that is identical to or complementary to a sequence in a target nucleic acid. In some instances, a molecular beacon probe sequence hybridizes to a sequence in an amplification product that is not identical to or complementary to a sequence in a target nucleic acid (e.g., hybridizes to a sequence added to an amplification product by way of a tailed amplification primer or ligation). Molecular beacons are highly specific and can discern a single nucleotide polymorphism. Molecular beacons also can be synthesized with different colored fluorophores and different target sequences, enabling simultaneous detection of several products in the same reaction (e.g., in a multiplex reaction). For quantitative amplification processes, molecular beacons can specifically bind to the amplified target following each cycle of amplification, and because non-hybridized molecular beacons are dark, it is not necessary to isolate the probe-target hybrids to quantitatively determine the amount of amplified product. The resulting signal is proportional to the amount of amplified product. Detection using molecular beacons can be done in real time or as an end-point detection method.

Detecting a nucleic acid amplification product can comprise use of lateral flow. Use of lateral flow typically includes use of a lateral flow device including but not limited to dipstick assays and thin layer chromatographic plates with various appropriate coatings. Immobilized on the flow path are various binding reagents for the sample, binding partners or conjugates involving binding partners for the sample and signal producing systems.

Detecting a nucleic acid amplification product can comprise use of FRET which is an energy transfer mechanism between two chromophores: a donor and an acceptor molecule. Briefly, a donor fluorophore molecule is excited at a specific excitation wavelength. The subsequent emission from the donor molecule as it returns to its ground state may transfer excitation energy to the acceptor molecule through a long range dipole-dipole interaction. The emission intensity of the acceptor molecule can be monitored and is a function of the distance between the donor and the acceptor, the overlap of the donor emission spectrum and the acceptor absorption spectrum and the orientation of the donor emission dipole moment and the acceptor absorption dipole moment. FRET can be useful for quantifying molecular dynamics, for example, in DNA-DNA interactions as described for molecular beacons. For monitoring the production of a specific product, a probe can be labeled with a donor molecule on one end and an acceptor molecule on the other. Probe-target hybridization brings a change in the distance or orientation of the donor and acceptor and FRET change is observed.

Detecting a nucleic acid amplification product can comprise use of fluorescence polarization (FP). FP techniques are based on the principle that a fluorescently labeled compound when excited by linearly polarized light will emit fluorescence having a degree of polarization inversely related to its rate of rotation. Therefore, when a molecule such as a tracer-nucleic acid conjugate, for example, having a fluorescent label is excited with linearly polarized light, the emitted light remains highly polarized because the fluorophore is constrained from rotating between the time light is absorbed and emitted. When a free tracer compound (i.e., unbound to a nucleic acid) is excited by linearly polarized light, its rotation is much faster than the corresponding tracer-nucleic acid conjugate and the molecules are more randomly oriented, therefore, the emitted light is depolarized. Thus, fluorescence polarization provides a quantitative means for measuring the amount of tracer-nucleic acid conjugate produced in an amplification reaction.

Detecting a nucleic acid amplification product can comprise use of surface capture, accomplished for example by the immobilization of specific oligonucleotides to a surface producing a biosensor that is both highly sensitive and selective. Example surfaces that can be used for attaching the probe include gold and carbon. Detecting a nucleic acid amplification product can comprise use of 5′ to 3′ exonuclease hydrolysis probes (e.g., TAQMAN). TAQMAN probes, for example, are hydrolysis probes that can increase the specificity of a quantitative amplification method (e.g., quantitative PCR). The TAQMAN probe principle relies on 1) the 5′ to 3′ exonuclease activity of Taq polymerase to cleave a dual-labeled probe during hybridization to a complementary target sequence and 2) fluorophore-based detection. A resulting fluorescence signal permits quantitative measurements of the accumulation of amplification product during the exponential stages of amplification, and the TAQMAN probe can significantly increase the specificity of the detection.

Detecting a nucleic acid amplification product can comprise use of intercalating and/or binding dyes, including dyes that specifically stain nucleic acid (e.g., intercalating dyes exhibit enhanced fluorescence upon binding to DNA or RNA). Dyes can include DNA or RNA intercalating fluorophores, including but not limited to, SYTO® 82, acridine orange, ethidium bromide, Hoechst dyes, PicoGreen®, propidium iodide, SYBR® I (an asymmetrical cyanine dye), SYBR® II, TOTO (a thiaxole orange dimer) and YOYO (an oxazole yellow dimer). Detecting a nucleic acid amplification product can comprise use of absorbance methods (e.g., colorimetric, turbidity). In some embodiments, detection and/or quantitation of nucleic acid can be achieved by directly converting absorbance (e.g., UV absorbance measurements at 260 nm) to concentration. Direct measurement of nucleic acid can be converted to concentration using the Beer Lambert law which relates absorbance to concentration using the path length of the measurement and an extinction coefficient. Detecting a nucleic acid amplification product can comprise use of electrophoresis (e.g., gel electrophoresis, capillary electrophoresis) and/or use of mass spectrometry. Mass Spectrometry is an analytical technique that can be used to determine the structure and quantity of a nucleic acid and can be used to provide rapid analysis of complex mixtures. Following amplification, samples can be ionized, the resulting ions separated in electric and/or magnetic fields according to their mass-to-charge ratio, and a detector measures the mass-to-charge ratio of ions. Mass spectrometry methods include, for example, MALDI, MALDI-TOF, and electrospray. These methods may be combined with gas chromatography (GC/MS) and liquid chromatography (LC/MS). Mass spectrometry (e.g., matrix-assisted laser desorption/ionization mass spectrometry (MALDI MS)) can be high throughput due to high-speed signal acquisition and automated analysis off solid surfaces.

Detecting a nucleic acid amplification product can comprise use of nucleic acid sequencing. The entire sequence or a partial sequence of an amplification product can be determined, and the determined nucleotide sequence may be referred to as a read. For example, linear amplification products may be analyzed directly without further amplification (e.g., by using single-molecule sequencing methodology). In some embodiments, linear amplification products is subject to further amplification and then analyzed (e.g., using sequencing by ligation or pyrosequencing methodology). Non-limiting examples of sequencing methods include single-end sequencing, paired-end sequencing, reversible terminator-based sequencing, sequencing by ligation, pyrosequencing, sequencing by synthesis, single-molecule sequencing, multiplex sequencing, solid phase single nucleotide sequencing, and nanopore sequencing. Detecting a nucleic acid amplification product can comprise use of digital amplification (e.g., digital PCR). Systems for digital amplification and analysis of nucleic acids are available (e.g., Fluidigm® Corporation).

### Lysis Buffers

### Lytic Agents

As disclosed herein, the lytic agents can comprise a detergent. The detergent can comprise one or more of a cationic surfactant, an anionic surfactant, a non-ionic surfactant, and an amphoteric surfactant. The anionic surfactant can comprise NH₄⁺, K⁺, Na⁺, or Li⁺ as a counter ion. The cationic surfactant can comprise I⁻, Br⁻, or Cl⁻ as a counter ion.

The lytic agents provided herein can be capable of acting as a denaturing agent. “Denaturing agent” or “denaturant,” as used herein, shall be given its ordinary meaning and include any compound or material which will cause a reversible unfolding of a protein. The strength of a denaturing agent or denaturant will be determined both by the properties and the concentration of the particular denaturing agent or denaturant. Suitable denaturing agents or denaturants include chaotropes, detergents, organic solvents, water miscible solvents, phospholipids, or a combination of two or more such agents. Suitable chaotropes include, but are not limited to, urea, guanidine, and sodium thiocyanate. Useful detergents may include, but are not limited to, strong detergents such as sodium dodecyl sulfate, or polyoxyethylene ethers (e.g. Tween or Triton detergents), Sarkosyl, mild non-ionic detergents (e.g., digitonin), mild cationic detergents (e.g., N->2,3-(Dioleyoxy)-propyl-N,N,N-trimethylammonium), mild ionic detergents (e.g. sodium cholate or sodium deoxycholate) or zwitterionic detergents including, but not limited to, sulfobetaines (Zwittergent), 3-(3-chlolamidopropyl)dimethylammonio-1-propane sulfate (CHAPS), and 3-(3-chlolamidopropyl)dimethylammonio-2-hydroxy-1-propane sulfonate (CHAPSO). Organic, water miscible solvents such as acetonitrile, lower alkanols (especially C2-C₄ alkanols such as ethanol or isopropanol), or lower alkandiols (especially C₂-C₄ alkandiols such as ethylene-glycol) may be used as denaturants. Phospholipids can be naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

Suitable surfactant levels can be from about 0.1% to about 25%, from about 0.25% to about 10%, or from about 0.5% to about 5% by weight of the total composition. In some embodiments, the surfactants are anionic surfactants, amphoteric surfactants, nonionic surfactants, zwitterionic surfactants, cationic surfactants, and mixtures thereof. In some embodiments, it can be advantageous to use anionic, amphoteric, nonionic and zwitterionic surfactants (and mixtures thereof).

Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type.

Suitable cationic surfactants can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; cetyl trimethylammonium bromide; di-isobutylphenoxyethyl-dimethylbenzylammonium chloride; coconut alkyltrimethyl-ammonium nitrite; cetyl pyridinium fluoride; etc. Certain cationic surfactants can also act as germicides in the compositions disclosed herein.

Suitable nonionic surfactants that can be used in the compositions, methods and kits of the present disclosure can be broadly defined as compounds produced by the condensation of alkylene oxide groups (hydrophilic in nature) with an organic hydrophobic compound which may be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials. These materials are useful for stabilizing foams without contributing to excess viscosity build for the consumer product composition.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilizing group, e.g., carboxy, sulphonate, sulphate, phosphate or phosphonate.

Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

### Reducing Agents

The lysis buffer and/or reagent composition (e.g., dried composition) can comprise one or more reducing agents. A “reducing agent” can be a compound or a group of compounds. As used herein, “reducing agent”, also known as “reductant,” “reducer,” or “reducing equivalent,” can refer to an element or compound that donates an electron to another species. In particular, a reducing agent is generally a compound that breaks disulfide bonds by reduction, thereby overcoming those tertiary protein folding and quaternary protein structures (oligomeric subunits) which are stabilized by disulfide bonds. Examples of a suitable reducing agent include, but are not limited to, 2-mercaptoethanol, DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid and HED. In some embodiments of the methods, compositions and kits provided herein, the lysis buffer and/or reagent composition (e.g., dried composition) does not comprise one or more reducing agents.

### Reagent Composition

The reagent compositions described herein (e.g., dried composition) can be provided in a “dry form,” or in a form not suspended in liquid medium. The “dry form” of the compositions can include dry powders, lyophilized compositions, spray-dried, or precipitated compositions. The “dry form” compositions can include one or more lyoprotectants, such as sugars and their corresponding sugar alcohols, such as sucrose, lactose, trehalose, dextran, erythritol, arabitol, xylitol, sorbitol, and mannitol; amino acids, such as arginine or histidine; lyotropic salts, such as magnesium sulfate; polyols, such as propylene glycol, glycerol, poly(ethylene glycol), or polypropylene glycol); and combinations thereof. Additional exemplary lyoprotectants include gelatin, dextrins, modified starch, and carboxymethyl cellulose. As used herein, the terms “lyophilization,” “lyophilized,” and “freeze-dried” refer to a process by which the material to be dried is first frozen and then the ice or frozen solvent is removed by sublimation in a vacuum environment. “Lyophilisate” refers to a lyphophilized substance.

The reagent composition (e.g., dried composition) can be frozen or lyophilized or spray dried. The reagent composition can be heat dried. The reagent composition can comprise one or more additives (e.g., an amino acid, a polymer, a sugar or sugar alcohol). The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof. Lyophilized reagents can include poly rA, EGTA, EDTA, Tween 80, and/or Tween 20.

The frozen or lyophilized or spray dried or heat dried composition or the aqueous composition for preparing the frozen or lyophilized or spray dried composition may comprise one or more of the following: (i) Non-aqueous solvents such as ethylene glycol, glycerol, dimethylsulphoxide, and dimethylformamide. (ii) Surfactants such as Tween 80, Brij 35, Brij 30, Lubrol-px, Triton X-10; Pluronic F127 (polyoxyethylene-polyoxypropylene copolymer) also known as poloxamer, poloxamine, and sodium dodecyl sulfate. (iii) Dissacharides such as trehalose, sucrose, lactose, and maltose. (iv) Polymers (which may have different MWs) such as polyethylene glycol, dextran, polyvinyl alcohol), hydroxypropyl methylcellulose, gelatin, polyvinylpyrrolidone, hydroxyethyl cellulose, Ficoll, and albumin. (v) Amino acids such as glycine, proline, 4-hydroxyproline, L-serine, glutamate, alanine, lysine, sarcosine, and gamma-aminobutyric acid.

The reagent composition (e.g., dried composition) can comprise one or more protectants and one or more amplification reagents. The one or more protectants can comprise a cyclodextrin compound. Cyclodextrins (CD) can be employed for complexation with lytic agents (e.g., SDS). Cyclodextrins (CDs) can be cyclic oligosaccharides which resemble truncated cones with hydrophobic inner cavity and hydrophilic outer surface The most commonly used natural cyclodextrins include 6, 7, and 8 glucose units, named as α, β and γ-CD. Natural CDs have can have solubility. Chemical modified CDs such as hydroxypropyl derivatives improve solubility up to 50% in aqueous media. CAVASOL® is the trade name of WACKER's cyclodextrin derivatives, which covers a variety of α, β and γ-CD derivatives. β-CD can form a strong inclusion complex (more so than α-CD and β-CD) with sodium dodecyl sulfate (SDS) in a predominately 1:1 stoichiometry. The binding constant of β-CD to SDS can range from 2100 M⁻¹ to 2500 M⁻¹.

### Kits

There are provided, in some embodiments, kits for detecting a target nucleic acid sequence in a sample. In some embodiments, the kit comprises: a signal-generating oligonucleotide disclosed herein. The kit can comprise: a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent, and wherein the detergent comprises one or more of a cationic surfactant, an anionic surfactant, a non-ionic surfactant, and an amphoteric surfactant. The kit can comprise: a reagent composition comprising one or more amplification reagents comprising one or more components for amplifying the target nucleic acid sequence under isothermal amplification conditions, wherein said one or more components for amplifying comprise: (i) a forward primer and a reverse primer, wherein the forward primer is capable of hybridizing to a sequence of a first strand of the target nucleic acid sequence, and the reverse primer is capable of hybridizing to a sequence of a second strand of the target nucleic acid sequence; and/or (ii) an enzyme having a hyperthermophile polymerase activity capable of generating a nucleic acid amplification product. In some embodiments, the reagent composition comprises a reverse transcriptase and/or a reverse transcription primer.

The kit can comprise: at least one component providing real-time detection activity for a nucleic acid amplification product. The real-time detection activity can be provided by a molecular beacon. The reagent composition (e.g., dried composition) can comprise a reverse transcriptase and/or a reverse transcription primer.

The molar ratio of the one or more protectants to the one or more amplification reagents can be between about 10:1 to about 1:10 (e.g., about 2:1). In some embodiments, the one or more additives comprise Tween 20, Triton X-100, Tween 80, a non-ionic detergent (e.g., a non-ionic surfactant), or any combination thereof. In some embodiments, the one or more protectants comprises a cyclodextrin compound. In some embodiments, the one or more lytic reagents comprise about 0.001% (w/v) to about 1.0% (w/v) (e.g., about 0.2% (w/v)) of the treated sample. In some embodiments, the one or more lytic agents comprise a detergent. The detergent can comprise one or more of a cationic surfactant, an anionic surfactant, a non-ionic surfactant, and an amphoteric surfactant. In some embodiments, it can be advantageous that the one or more protectants are capable of sequestering the one or more lytic agents, thereby preventing the denaturing of the one or more amplification reagents by the one or more lytic agents.

Kits can comprise, for example, one or more polymerases and one or more primers, and optionally one or more reverse transcriptases and/or reverse transcription primers, as described herein. Where one target is amplified, a pair of primers (forward and reverse) can be included in the kit. Where multiple target sequences are amplified, a plurality of primer pairs can be included in the kit. A kit can include a control polynucleotide, and where multiple target sequences are amplified, a plurality of control polynucleotides can be included in the kit.

The enzyme having a hyperthermophile polymerase activity can have an amino acid sequence that is at least about 90% or 95% identical to the amino acid sequence of SEQ ID NO: 7 or a functional fragment thereof. For example, the enzyme having a hyperthermophile polymerase activity can comprise the amino acid sequence of SEQ ID NO: 7.

The nucleic acid amplification product can be about 20 to 40 bases long. The nucleic acid amplification product can comprise: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The biological entities can comprise one or more of prokaryotic cells, eukaryotic cells, viral particles, exosomes, protoplasts, and microvesicles. The biological entities can comprise a virus, a bacteria, a fungi, a protozoa, portions thereof, or any combination thereof. The target nucleic acid sequence can be a nucleic acid sequence of a virus, bacteria, fungi, or protozoa. The sample nucleic acids can be derived from a virus, bacteria, fungi, or protozoa.

Kits can also comprise one or more of the components in any number of separate vessels, chambers, containers, packets, tubes, vials, microtiter plates and the like, or the components can be combined in various combinations in such containers. Components of the kit can, for example, be present in one or more containers. In some embodiments, all of the components are provided in one container. In some embodiments, the enzymes (e.g., polymerase(s) and/or reverse transcriptase(s)) can be provided in a separate container from the primers. The components can, for example, be lyophilized, heat dried, freeze dried, or in a stable buffer. In some embodiments, polymerase(s) and/or reverse transcriptase(s) are in lyophilized form or heat dried form in a single container, and the primers are either lyophilized, heat dried, freeze dried, or in buffer, in a different container. In some embodiments, polymerase(s) and/or reverse transcriptase(s), and the primers are, in lyophilized form or heat dried form, in a single container.

Kits can further comprise, for example, dNTPs used in the reaction, or modified nucleotides, vessels, cuvettes or other containers used for the reaction, or a vial of water or buffer for re-hydrating lyophilized or heat-dried components. The buffer used can, for example, be appropriate for both polymerase and primer annealing activity.

Kits can also comprise instructions for performing one or more methods described herein and/or a description of one or more components described herein. Instructions and/or descriptions can be in printed form and can be included in a kit insert. A kit also can include a written description of an internet location that provides such instructions or descriptions.

Kits can further comprise reagents used for detection methods, for example, reagents used for FRET, lateral flow devices, dipsticks, fluorescent dye, colloidal gold particles, latex particles, a molecular beacon, or polystyrene beads.

### EXAMPLES

Some aspects of the embodiments discussed above are disclosed in further detail in the following examples, which are not in any way intended to limit the scope of the present disclosure.

### Example 1

### FluB PB2 Assay: Non-Specific Product Formation (without 2′OM Protection)

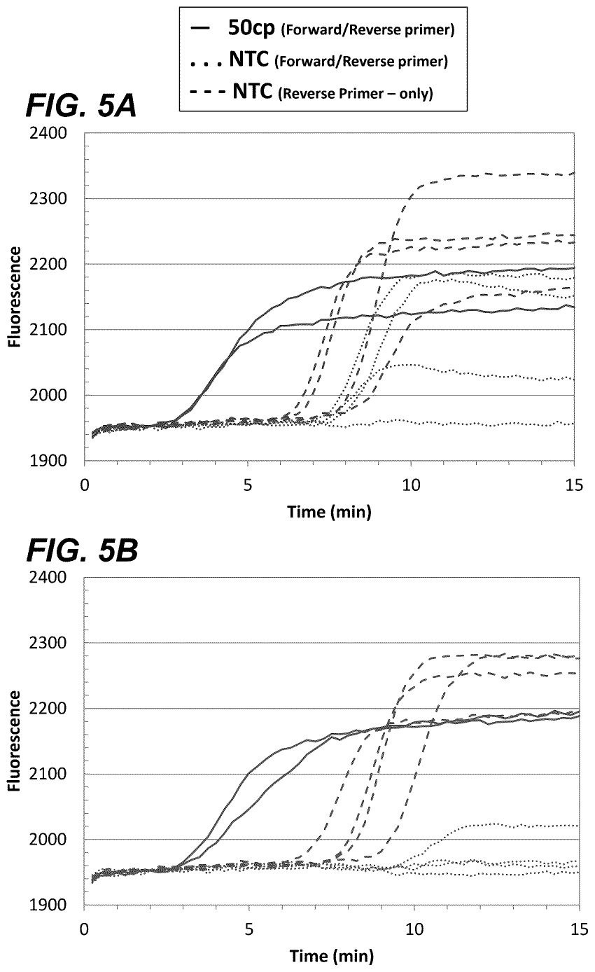

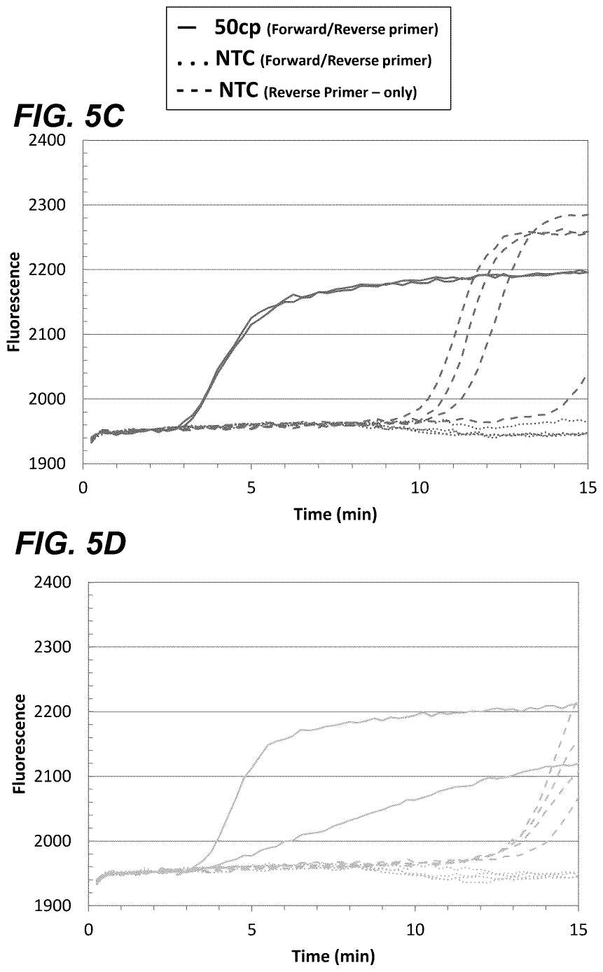

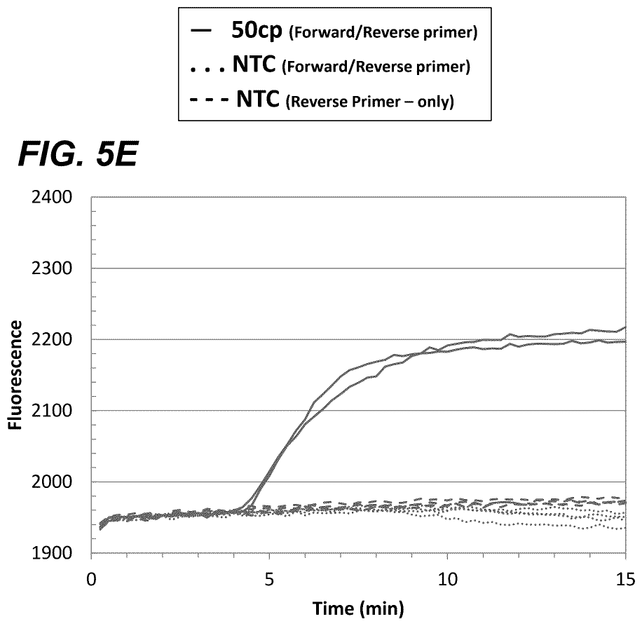

This example demonstrates that non-specific product formation between the reverse primer and molecular beacon can produce a false-positive signal in the absence of the assay target sequence. FIGS. 5A-5E depict data showing a concentration-dependent trend between the FluB PB2 Reverse Primer and the occurrence of false-positive signals in No-Target Conditions (NTC). FIGS. 5A-5E depict data showing FluB PB2 non-specific product formation (without 2′OM protection). The FluB PB2 Assay Primer and Probe are shown in Table 4. The FluB PB2 reverse primer was titrated to 600 nM (FIG. 5A), 500 nM (FIG. 5B), 400 nM (FIG. 5C), 300 nM (FIG. 5D), and 200 nM (FIG. 5E). As the concentration of the Reverse Primer increases, the rate of false-positive NTCs increases and detection time for these NTCs occurs earlier.

TABLE 4
FluB PB2 Assay Primer and Probe

| Name | Sequence (5′-3′) | SEQ ID NO |
|---|---|---|
| Reverse Primer | C C CATATTCC*AA | 41 |
| Molecular Beacon | AGC AC+C +CATATTCC AAT+C TT+C +GG+G | 42 |

* indicates phosphorothioated DNA bases (e.g. A*, G*, C*, T*)
* denotes phosphorothioate linkage between adjacent bases
+ indicates locked nucleic acids (e.g. +A, +G, +C, +T)
Underline indicates Spacer Region

### Example 2

### FluB PB2 Assay: Prevention of Non-Specific Product Formation Via 2′OM Protection

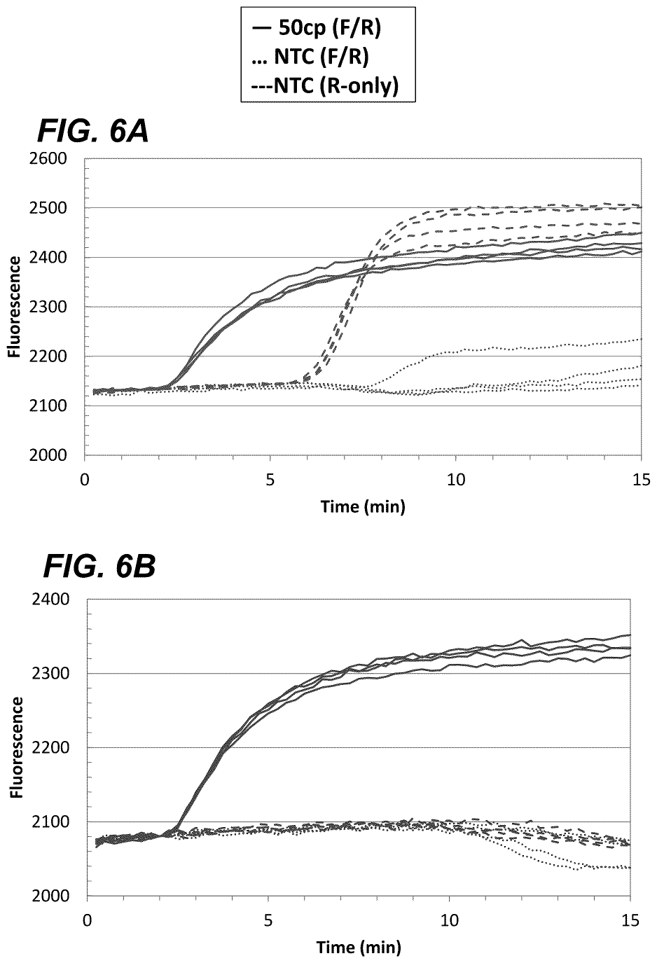

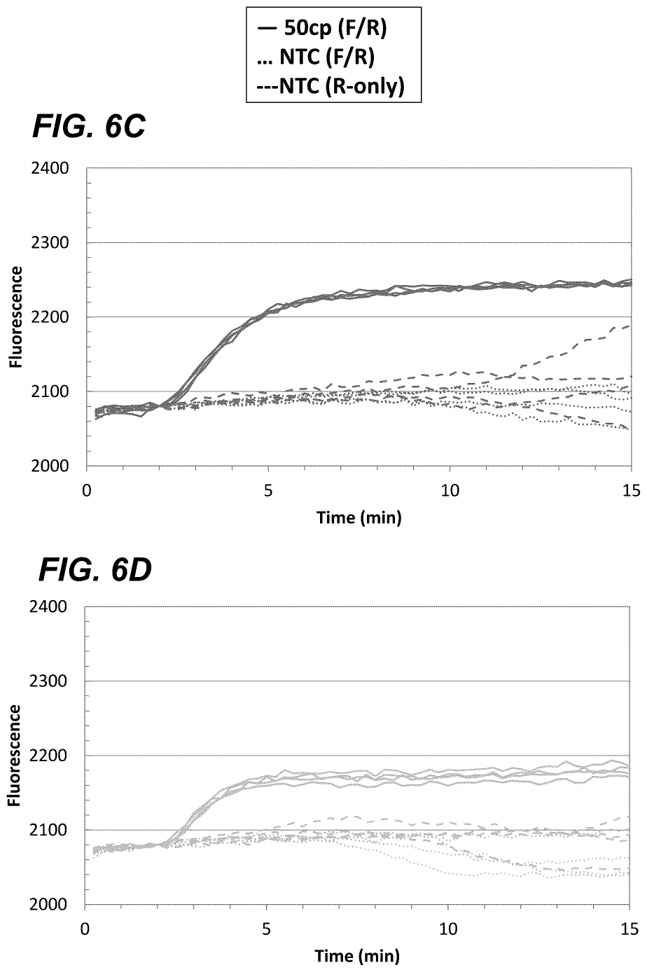

This example demonstrates prevention of unintended extension-product formation mitigates false-positive signals caused by read-through of the primer. FIGS. 6B-6D depict the results of employing three variations of FluB PB2 molecular beacons that include a 2′OM modification. These molecular beacons are observed to have significantly less false-positive signals in No-Target Conditions (NTCs) as compared to the Non-Modified, Control beacon (FIG. 6A). FIGS. 6A-6D depict data related to prevention of FluB PB2 Assay non-specific product formation via 2′OM-modified beacons. Results generated using non-modified control molecular beacon (FIG. 6A; LNA3.13), 2′OM-modified version 1 (FIG. 6B; LNA3.13 ml), 2′OM-modified version 2 (FIG. 6C; LNA3.13m2), and 2′OM-modified version 3 (FIG. 6D; LNA3.13m3) are depicted. In some embodiments, the probes (e.g., molecular beacons) provided herein comprise a 5′ modification (e.g., 5HEX). In some embodiments, the probes (e.g., molecular beacons) provided herein comprise a 3′ modification (e.g., 3IAbRQSp).

TABLE 5
FluB PB2 Assay Probes

| Name | Sequence (5′-3′) | SEQ ID NO |
|---|---|---|
| Control (non-modified) | AGC AC+C +CATATTCC AAT+C TT+C +GG+G | 42 |
| Molecular Beacon | | |
| (LNA3.13) | | |
| 2′OM-modified | agcaC+C+CATATTCCAmAT+CTT+C+gg+g | 43 |
| Molecular Beacon | | |
| (B LNA3.13ml) | | |
| 2′OM-modified | agcaC+C+CATATTCmCAAT+CTT+C+gg+g | 44 |
| Molecular Beacon | | |
| (B LNA3.13m2) | | |
| 2′OM-modified | agcaC+C+CATATTCmCAmAT+CTT+C+gg+g | 45 |
| Molecular Beacon | | |
| B LNA3.13m3) | | |

m indicates 2′ O-Methyl base (e.g. mA, mG, mC, mU)
Underline indicates Spacer Region
+ indicates locked nucleic acids (e.g. +A, +G, +C, +T)

### Example 3

### Neisseria Gonorrhea (“NG”, “GC”) Assay & Chlamydia trachomatis (“CT”) Assay False Positive Assessment

This assay demonstrates the prevention of false positives in Neisseria Gonorrhea and Chlamydia trachomatis assays via the protected probes provided herein.

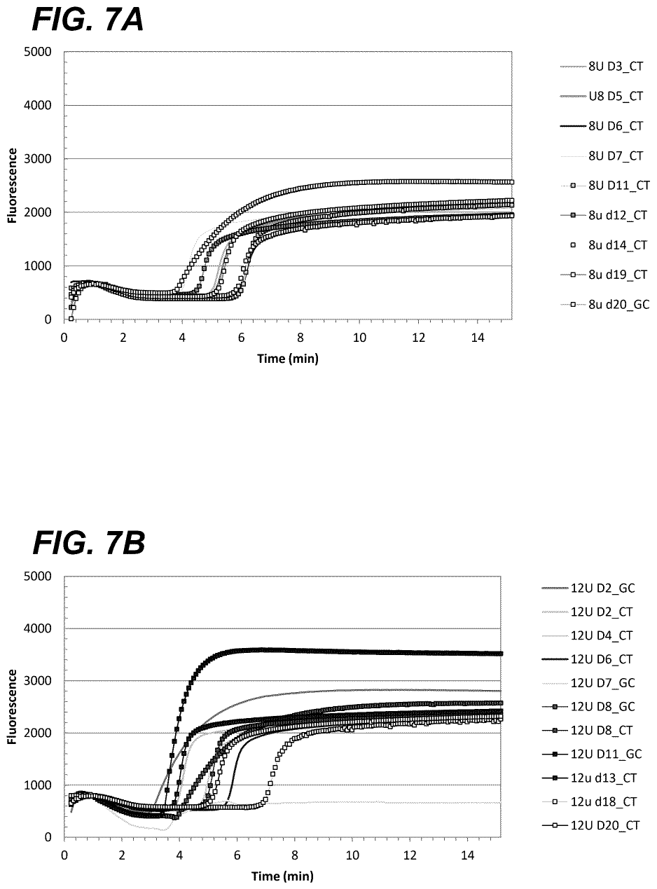

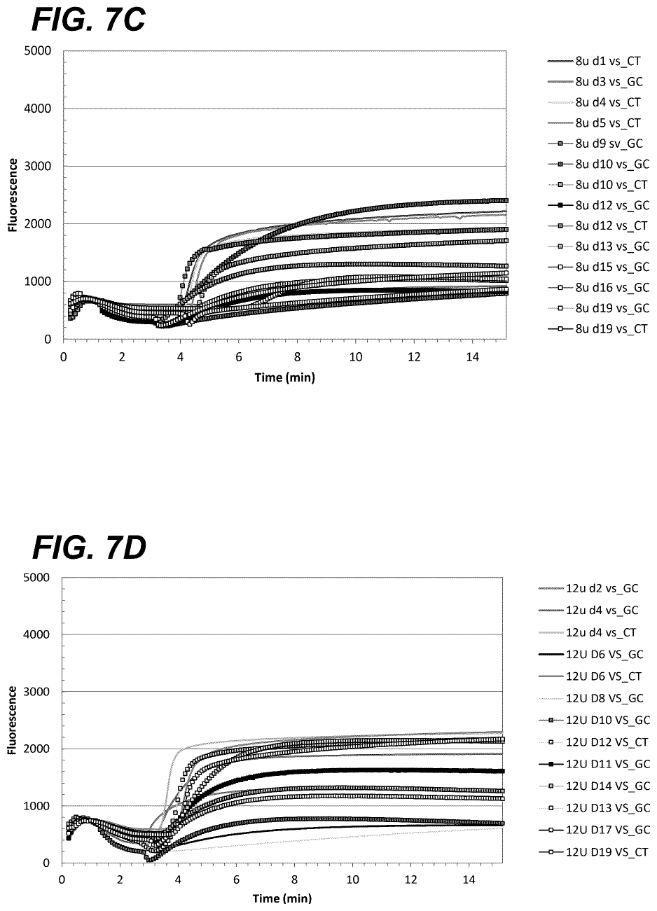

A false positive assessment was first conducted using 20 urine samples (10% in 1.11×GRBS) and 20 ProbeTec™ swabs (1.67% in 1×GRBS) using unmodified CT and GC ROX beacons. FIGS. 7A-7D depict data related to false positive assessment in Neisseria Gonorrhea and Chlamydia trachomatis assays conducted with NTC 10% urine samples (FIGS. 7A-7B) and NTC vaginal swab samples (FIGS. 7C-7D) using 8U 9dN polymerase (FIG. 7A, FIG. 7C) and 12U 9dN polymerase (FIG. 7B, FIG. 7D). Tables 6 and 7 depict the results of testing NTC urine samples and NTC vaginal swab samples, respectively, with different amounts of 9dN polymerase.

TABLE 6
False Positive Assessment - 10% Urine

| 9dN | 8U | 12U |
|---|---|---|
| Neisseria Gonorrhea Assay | 1/20 (5%) | 4/20 (20%) |
| Chlamydia trachomatis Assay | 8/20 (40%) | 7/20 (35%) |

TABLE 7
False Positive Assessment - 1.67% Vaginal Matrix

| 9dN | 8U | 12U |
|---|---|---|
| Neisseria Gonorrhea Assay | 8/20 (40%) | 9/20 (45%) |
| Chlamydia trachomatis Assay | 6/20 (30%) | 4/20 (20%) |

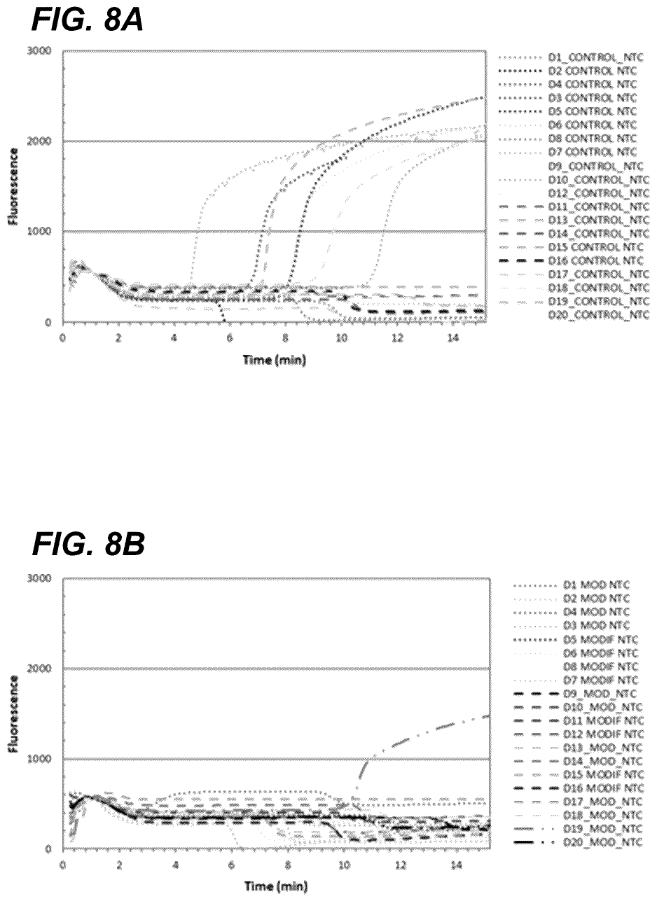

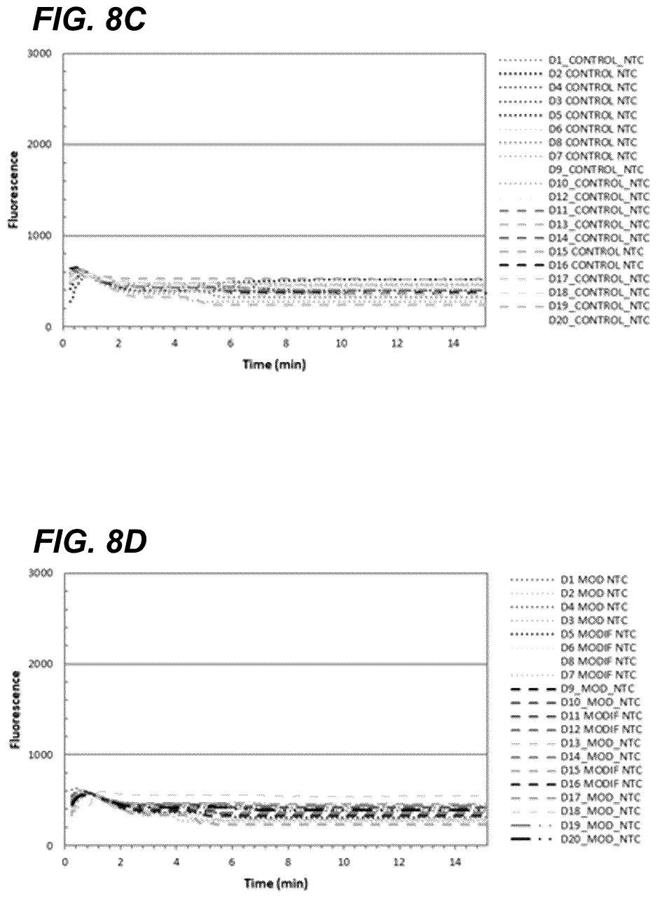

Next, a false positive comparison of nominal versus modified (protected) probes was conducted with NTC urine samples (N=20). FIGS. 8A-8D depict data related to false positive assessment in Chlamydia trachomatis assays (FIGS. 8A-8B) and Neisseria Gonorrhea assays (FIGS. 8C-8D) conducted with NTC 15% urine samples using nominal probes (FIG. 8A, FIG. 8C) and protected probes (FIG. 8B, FIG. 8D). The use of the protected probes led to a reduction in false positives from 7/20 (35%) to 1/20 (5%) in the Chlamydia trachomatis assay, with the single remaining false positive attributed to suspected target template contamination. Thus, this higher false positive frequency was mitigated by O-methyl modification of Beacon. In the Neisseria Gonorrhea assays the same performance was seen between the two probes (as no false positives were observed).

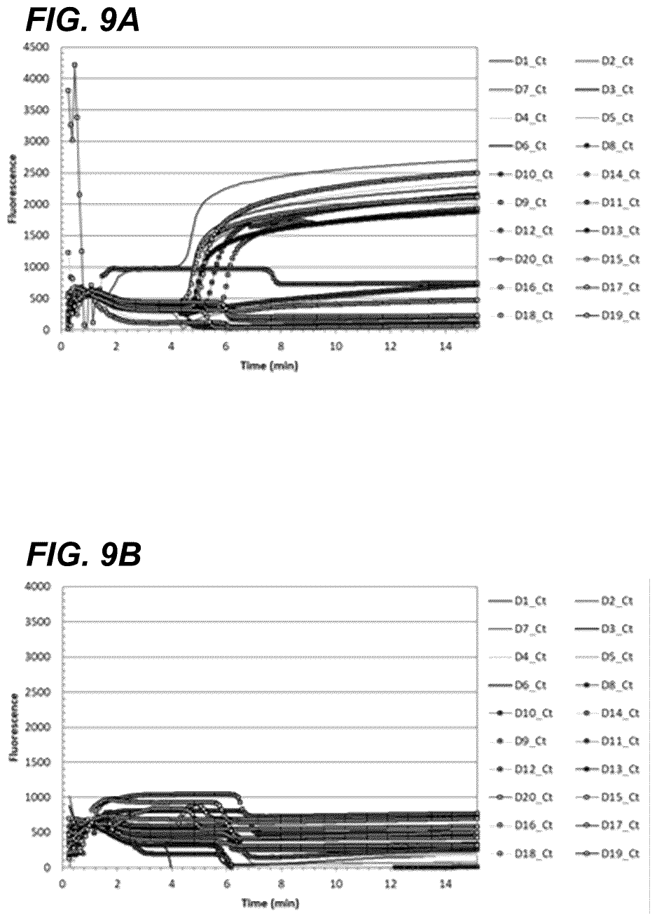

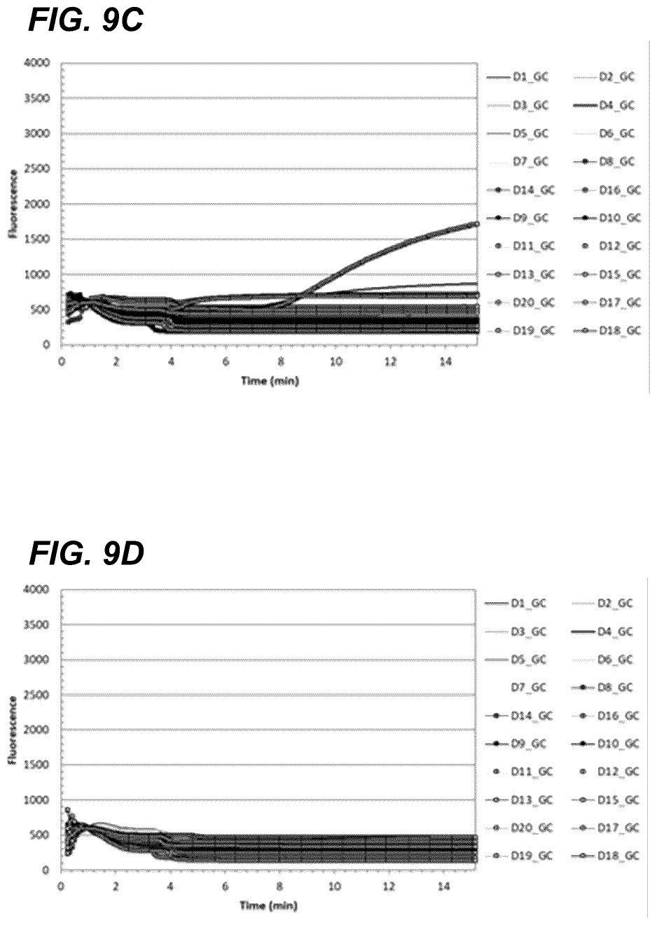

The false positive comparison of nominal versus modified (protected) probes was then conducted with vaginal swabs (N=20). FIGS. 9A-9D depict data related to false positive assessment in Chlamydia trachomatis assays (FIGS. 9A-9B) and Neisseria Gonorrhea assays (FIGS. 9C-9D) conducted with vaginal swab samples using nominal probes (FIG. 9A, FIG. 9C) and protected probes (FIG. 9B, FIG. 9D). A higher number of false positives was observed with vaginal swab samples, mitigated by O-methyl modification of beacons for both assays. The use of the protected probes led to a reduction in false positives from 9/20 (45%) to 0/20 (0%) in the Chlamydia trachomatis assay. The use of the protected probes led to a reduction in false positives from 1/20 (5%) to 0/20 (0%) in the Neisseria Gonorrhea assay.

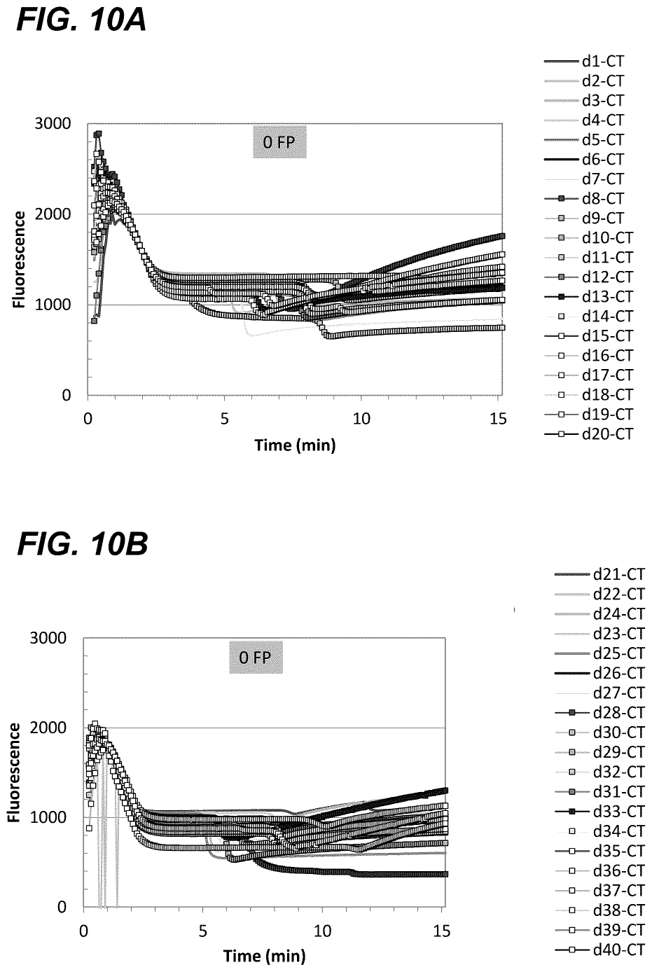

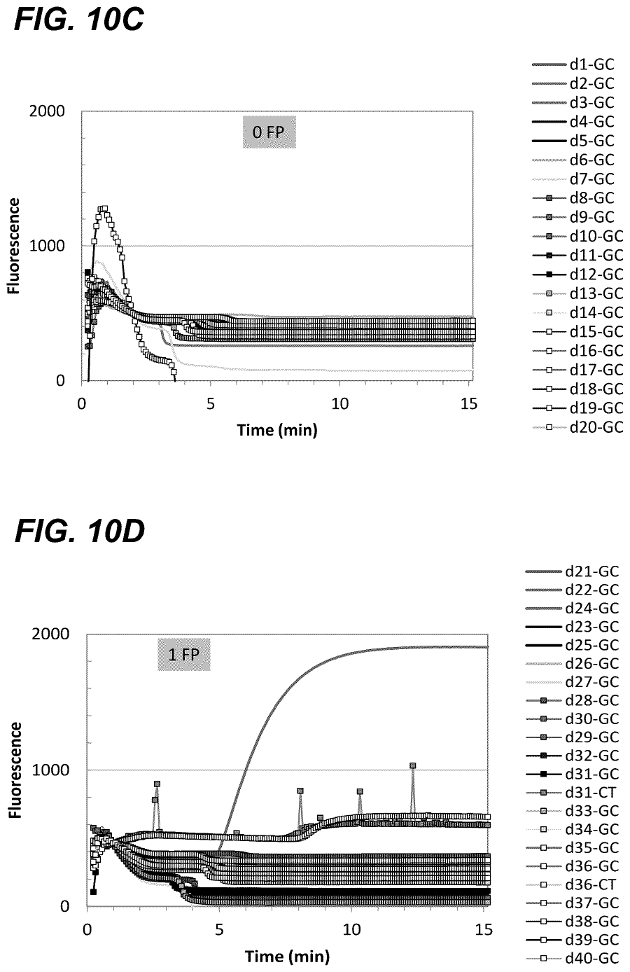

NTC testing was next performed with modified probes using NTC 10% urine samples (N=40). This assay switched to HEX beacon for Chlamydia trachomatis (O-methyl modified), and 0% FP was observed for Chlamydia trachomatis and 2.5% for Neisseria Gonorrhea (ROX). FIGS. 10A-10D depict data related to false positive assessment in Chlamydia trachomatis assays (FIGS. 10A-10B) and Neisseria Gonorrhea assays (FIGS. 10C-10D) conducted with NTC urine samples and using protected probes.

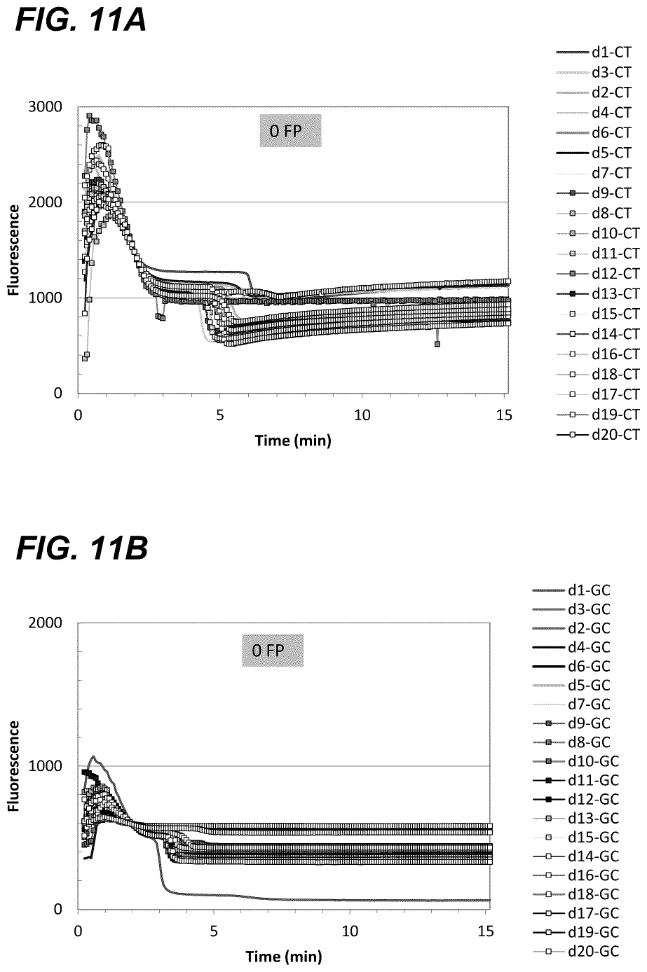

NTC testing was next performed with modified probes using NTC vaginal swab samples (1.67% VM (N=20)). 3-in-1 Swabs were expressed in 1×GRBS (1.67%) and the assays were conducted using CT HEX (2′ O-methyl modified beacon and GC ROX (2′ O-methyl modified). FIGS. 11A-11B depict data related to depict data related to false positive assessment in Chlamydia trachomatis assays (FIG. 11A) and Neisseria Gonorrhea assays (FIG. 11B) conducted with NTC vaginal swab samples and using protected probes. Zero percent FP was seen for both assays.

### Example 4

### FluA Assay: RNA Modified Beacons

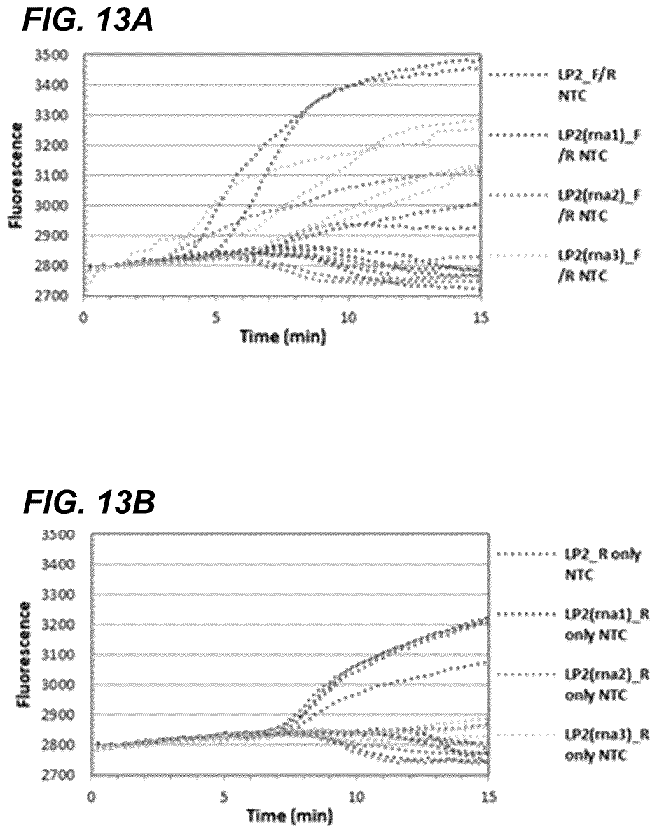

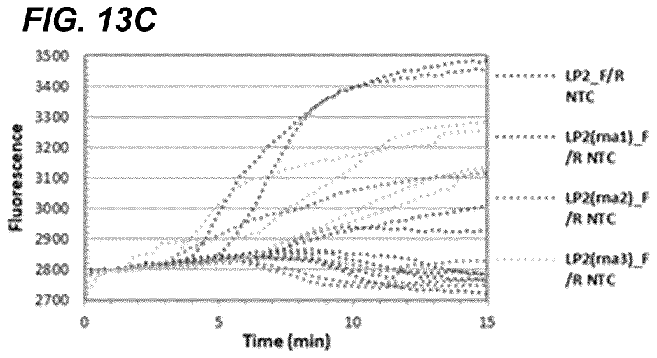

This example demonstrates the use of RNA base incorporation for protection of molecular beacons. FIGS. 13A-13C depict data related to RNA base incorporation for protection of molecular beacons in FluA assays. Assays were performed with NTC only-Primer Drop (FIGS. 13A-13B) or probes were screened with target (FIG. 13C). In forward primer and reverse primer conditions, an RNA modified probe (LP2(rna3)) had same rate of false-positives as a nominal probe (LP2), no false-positives detected by RNA modified probe LP2(rna1), and 1 out of 4 false-positives detected by RNA modified probe LP2(rna2) (FIG. 14A). In reverse primer only conditions RNA modifications to the probe helped to mitigate false-positive detection (FIG. 13B). When probes were screened with target (FIG. 13C) RNA modified probe LP2(rna3) had same FP rate of as nominal probe LP2 (3 out of 4 NTCs detected). RNA modifications were shown to negatively impact binding of target product to probe (absence of false positives seen by LP2(rna1) and LP2(rna2) may be due to this). This example demonstrates RNA base modification of probes can offer protection against non-specific product formation.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for detecting a target nucleic acid sequence in a sample, comprising:
amplifying a target nucleic acid sequence in an amplification reaction mixture under an isothermal amplification condition, thereby generating a nucleic acid amplification product; and
detecting the nucleic acid amplification product with a signal-generating oligonucleotide, wherein the signal-generating oligonucleotide is capable of hybridizing to the nucleic acid amplification product and comprises one or more polymerase stoppers.

2. The method of claim 1, comprising:
contacting a sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence; and
contacting a reagent composition with the treated sample to generate the amplification reaction mixture, wherein the reagent composition comprises one or more amplification reagents.

3. The method of any one of claims 1-2, wherein:
the signal-generating oligonucleotide comprises a 5′ subdomain and a 3′ subdomain,
the signal-generating oligonucleotide comprises a loop domain situated between the 5′ subdomain and the 3′ subdomain,
intramolecular nucleotide base pairing between the 5′ subdomain and the 3′ subdomain are capable of forming a paired stem domain,
the one or more polymerase stoppers are situated in the loop domain, and
the 5′ subdomain, the paired stem domain, and/or the 3′ subdomain does not comprise the one or more polymerase stoppers.

4. The method of any one of claims 1-3, wherein the nucleic acid amplification product comprises:
(1) the sequence of a forward primer, and the reverse complement thereof,
(2) the sequence of a reverse primer, and the reverse complement thereof, and
(3) a spacer sequence flanked by (1) the sequence of the forward primer and the reverse complement thereof and (2) the sequence of the reverse primer and the reverse complement thereof, wherein the spacer sequence is 1 to 10 bases long.

5. The method of any one of claims 1-4, wherein:
the signal-generating oligonucleotide comprises a first region comprising the sequence of at least a portion of the reverse primer;
the signal-generating oligonucleotide comprises a second region comprising a sequence complementary to at least a portion of the forward primer; and/or
the signal-generating oligonucleotide comprises a spacer region comprising the sequence of at least a portion of the spacer sequence,
optionally, the first region, the second region, and/or the spacer region comprises one or more polymerase stoppers.

6. The method of any one of claims 1-5, wherein:
the first region comprises a sequence complementary to at least two 3′ terminal nucleotides of the forward primer and/or the reverse primer;
the second region comprises a sequence complementary to at least two 3′ terminal nucleotides of the forward primer and/or the reverse primer; and/or
the spacer region comprises a sequence complementary to at least two 3′ terminal nucleotides of the forward primer and/or the reverse primer.

7. The method of any one of claims 1-6, wherein:
the signal-generating oligonucleotide is about 10 nucleotides to about 100 nucleotides in length;
the forward primer and/or the reverse primer is about 5 nucleotides to about 25 nucleotides in length;
the second region, the spacer region, and/or the first region is about 1 nucleotide to about 25 nucleotides in length; and/or
the 5′ subdomain, the 3′ subdomain, the loop domain, the 5′ terminal domain, and/or the 3′ terminal domain is about 1 nucleotide to about 25 nucleotides in length.

8. The method of any one of claims 1-7, wherein:
the signal-generating oligonucleotide comprises a 5′ terminal domain situated 5′ of the 5′ subdomain, and/or
the signal-generating oligonucleotide comprises a 3′ terminal domain situated 3′ of the 5′ subdomain,
optionally the 5′ terminal domain and/or the 3′ terminal domain does not comprise the one or more polymerase stoppers.

9. The method of any one of claims 1-8, wherein:
the first region comprises at least a portion of the 5′ subdomain and/or loop domain,
the spacer region comprises at least a portion of the loop domain, and
the second region comprises at least a portion of the loop domain and/or 3′ subdomain.

10. The method of any one of claims 1-9, wherein the one or more polymerase stoppers comprise one or more 2′-O-methyl (2′OM) RNA nucleotides.

11. The method of any one of claims 1-10, wherein the one or more polymerase stoppers comprise one or more of an abasic site, a stable abasic site, a chemically trapped abasic site, or any combination thereof.

12. The method of claim 11, wherein:
the stable abasic site comprises 1′,2′-dideoxy;
the chemically trapped abasic site comprises an abasic site reacted with alkoxy amine or sodium borohydride;
the abasic site comprises an apurinic site, an apyrimidinic site, or both; and/or
the abasic site is generated by an alkylating agent or an oxidizing agent.

13. The method of any one of claims 1-12, wherein the one or polymerase stoppers comprise:
one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-m ethyl cytidines, one or more 5-hydroxymethylcytidines, one or more 2′-O-Methyl RNA bases, one or more unmethylated RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(o)NHCH₂—C₆H₃NO₂—CH(CH₃)OPO₃] groups, one or more hexandiol groups, one or more spacer 9 (iSp9) [(OCH₂CH₂)₃OPO₃] groups, one or more spacer 18 (iSp18) [(OCH₂CH₂₆OPO₃] groups, or any combination thereof.

14. The method of any one of claims 1-13, wherein the signal-generating oligonucleotide comprises one or more phosphorothioate linkages and/or one or more locked nucleic acids.

15. The method of any one of claims 1-14, wherein the signal-generating oligonucleotide is a TaqMan detection probe oligonucleotide, a molecular beacon detection probe oligonucleotide, or a molecular torch detection probe oligonucleotide.

16. The method of any one of claims 1-15, wherein the signal-generating oligonucleotide comprises a label, optionally the label comprises a quenchable label, further optionally the quenchable label is a fluorophore.

17. The method of any one of claims 1-16, wherein the signal-generating oligonucleotide comprises a quencher, optionally:
the label is situated in the 3′ terminal domain and the quencher is situated in the 5′ terminal domain, and/or
the label is situated in the 5′ terminal domain and the quencher is situated in the 3′ terminal domain.

18. The method of any one of claims 1-17,
wherein, upon the forward primer binding the signal-generating oligonucleotide to form a first undesirable duplex, the one or more polymerase stoppers are capable of stopping polymerase extension of the forward primer of the first undesirable duplex to the 5′ end of the signal-generating oligonucleotide,
optionally, the one or more polymerase stoppers are capable of stopping polymerase extension of the forward primer of the first undesirable duplex beyond the one or more polymerase stoppers of the signal-generating oligonucleotide.

19. The method of any one of claims 1-18,
wherein, upon the reverse primer binding the signal-generating oligonucleotide to form a second undesirable duplex, the one or more polymerase stoppers are capable of stopping polymerase extension of the reverse primer of the second undesirable duplex to the 5′ end of the signal-generating oligonucleotide,
optionally, the one or more polymerase stoppers are capable of stopping polymerase extension of the reverse primer of the second undesirable duplex beyond the one or more polymerase stoppers of the signal-generating oligonucleotide.

20. The method of any one of claims 1-19,
wherein, upon an extraneous nucleic acid binding the signal-generating oligonucleotide to form a third undesirable duplex, the one or more polymerase stoppers are capable of stopping polymerase extension of the extraneous nucleic acid of the third undesirable duplex to the 5′ end of the signal-generating oligonucleotide,
optionally, the one or more polymerase stoppers are capable of stopping polymerase extension of the extraneous nucleic acid of the third undesirable duplex beyond the one or more polymerase stoppers of the signal-generating oligonucleotide,
optionally, the extraneous nucleic acid is selected from the group comprising a sample nucleic acid, a primer configured to hybridize a second target nucleic acid sequence, a primer configured to hybridize an internal control, or any combination thereof.

21. The method of any one of claims 1-20, wherein the sample nucleic acids comprise a nucleic acid comprising the target nucleic acid sequence.

22. The method of any one of claims 1-21, wherein amplifying the target nucleic acid sequence comprises:
amplifying a target nucleic acid sequence comprising a first strand and a second strand complementary to each other in an isothermal amplification condition, wherein the amplifying comprises contacting a nucleic acid comprising the target nucleic acid sequence with:
i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and
ii) an enzyme having a hyperthermophile polymerase activity, thereby generating the nucleic acid amplification product.

23. The method of any one of claims 21-22, wherein the nucleic acid is a double-stranded DNA.

24. The method of any one of claims 21-23, wherein the nucleic acid is a product of reverse transcription reaction, optionally the nucleic acid is a product of reverse transcription reaction generated from sample ribonucleic acids, further optionally the amplifying comprises generating the nucleic acid by a reverse transcription reaction.

25. The method of any one of claims 1-24, wherein the sample nucleic acids comprise sample ribonucleic acids, and wherein the method comprises contacting sample ribonucleic acids with a reverse transcriptase and/or a reverse transcription primer to generate a cDNA.

26. The method of any one of claims 1-25, wherein amplifying the target nucleic acid sequence comprises:
(c1) contacting sample ribonucleic acids with a reverse transcriptase and/or a reverse transcription primer to generate a cDNA;
(c2) contacting the cDNA with an enzyme having a hyperthermophile polymerase activity to generate a double-stranded DNA (dsDNA), wherein the dsDNA comprises a target nucleic acid sequence, and wherein the target nucleic acid sequence comprises a first strand and a second strand complementary to each other;
(c3) amplifying the target nucleic acid sequence under an isothermal amplification condition, wherein the amplifying comprises contacting the dsDNA with:
(i) a forward primer and a reverse primer, wherein the forward primer is capable of hybridizing to a sequence of the first strand of the target nucleic acid sequence, and the reverse primer is capable of hybridizing to a sequence of the second strand of the target nucleic acid sequence; and
(ii) the enzyme having a hyperthermophile polymerase activity, thereby generating the nucleic acid amplification product.

27. The method of any one of claims 1-26,
wherein, if the forward primer binds the signal-generating oligonucleotide to form a first undesirable duplex, extension of the forward primer of the first undesirable duplex to the 5′ end of the signal-generating oligonucleotide by an enzyme having a hyperthermophile polymerase activity generates a first undesirable extension product,
wherein the first undesirable extension product is capable of being amplified by the enzyme having a hyperthermophile polymerase activity in the presence of the forward primer and the reverse primer to form a first undesirable amplification product; and
wherein the one or more polymerase stoppers are capable of stopping polymerase extension of the forward primer of the first undesirable duplex to generate a first stalled extension product,
wherein the first stalled extension product is not capable of being amplified by the enzyme having a hyperthermophile polymerase activity in the presence of the forward primer and reverse primer to generate the first undesirable amplification product.

28. The method of any one of claims 1-27, wherein the one or more polymerase stoppers are capable of stopping polymerase extension of the forward primer of the first undesirable duplex beyond the one or more polymerase stoppers of the signal-generating oligonucleotide.

29. The method of any one of claims 1-28,
wherein, if the reverse primer binds the signal-generating oligonucleotide to form a second undesirable duplex, extension of the reverse primer of the second undesirable duplex to the 5′ end of the signal-generating oligonucleotide by an enzyme having a hyperthermophile polymerase activity generates a second undesirable extension product,
wherein the second undesirable extension product is capable of being amplified by the enzyme having a hyperthermophile polymerase activity in the presence of the reverse primer to form a second undesirable amplification product; and
wherein the one or more polymerase stoppers are capable of stopping polymerase extension of the reverse primer of the second undesirable duplex to generate a second stalled extension product,
wherein the second stalled extension product is not capable of being amplified by the enzyme having a hyperthermophile polymerase activity in the presence of the reverse primer to generate the second undesirable amplification product.

30. The method of any one of claims 1-29, wherein the one or more polymerase stoppers are capable of stopping polymerase extension of the reverse primer of the second undesirable duplex beyond the one or more polymerase stoppers of the signal-generating oligonucleotide.

31. The method of any one of claims 1-30,
wherein, if the extraneous nucleic acid binds the signal-generating oligonucleotide to form a third undesirable duplex, extension of the extraneous nucleic acid of the third undesirable duplex to the 5′ end of the signal-generating oligonucleotide by an enzyme having a hyperthermophile polymerase activity generates a third undesirable extension product,
wherein the third undesirable extension product is capable of being amplified by the enzyme having a hyperthermophile polymerase activity in the presence of the reverse primer to form a third undesirable amplification product; and
wherein the one or more polymerase stoppers are capable of stopping polymerase extension of the extraneous nucleic acid of the third undesirable duplex to generate a third stalled extension product,
wherein the third stalled extension product is not capable of being amplified by the enzyme having a hyperthermophile polymerase activity in the presence of the reverse primer to generate the third undesirable amplification product.

32. The method of any one of claims 1-31, wherein the one or more polymerase stoppers are capable of stopping polymerase extension of the extraneous nucleic acid of the third undesirable duplex beyond the one or more polymerase stoppers of the signal-generating oligonucleotide.

33. The method of any one of claims 1-32, wherein the detecting step comprises contacting the nucleic acid amplification product with the signal-generating oligonucleotide for hybridization.

34. The method of any one of claims 1-33, wherein detecting the nucleic acid amplification product comprises use of a real-time detection method.

35. The method of any one of claims 1-34, wherein:
the label is capable of generating a signal upon the signal-generating oligonucleotide hybridizing the nucleic acid amplification product; and/or
upon the signal-generating oligonucleotide hybridizing the nucleic acid amplification product, the label generates a signal, optionally the signal is fluorescence.

36. The method of any one of claims 1-35, wherein:
the detecting step comprises detecting the signal of the label before the amplification reaction, after the amplification reaction, or both;
detecting the nucleic acid amplification product comprises detecting a signal generated by the label of the signal-generating oligonucleotide, optionally the label is a fluorophore and the signal is fluorescence;
detecting a signal comprises detecting fluorescence emitted by the label;
the method comprises determining the presence, absence and/or amount of the target nucleic acid sequence in the sample;
determining the presence, absence and/or amount of the target nucleic acid sequence in the sample comprises determining the presence, absence and/or amount of the dsDNA and/or nucleic acid that comprises the target nucleic acid sequence in the sample;
the presence, absence and/or amount of the signal indicates the presence, absence and/or amount of the target nucleic acid sequence in the sample; and/or
the presence, absence and/or amount of the signal indicates the presence, absence and/or amount of the dsDNA and/or nucleic acid that comprises the target nucleic acid sequence in the sample.

37. The method of any one of claims 1-36, wherein:
the label is capable of generating a false positive signal upon the signal-generating oligonucleotide hybridizing the first undesirable amplification product, the second undesirable amplification product, and/or the third undesirable amplification product, optionally the signal and the false positive signal are indistinguishable; and/or
upon the signal-generating oligonucleotide hybridizing the first undesirable amplification product, the second undesirable amplification product, and/or the third undesirable amplification product, the label generates a false positive signal, optionally the signal and the false positive signal are indistinguishable.

38. The method of any one of claims 1-37, wherein:
the generation of the first undesirable amplification product, the second undesirable amplification product, and/or the third undesirable amplification product reduces the likelihood of an accurate determination of the presence, absence and/or amount of the target nucleic acid sequence in the sample; and/or
the detection of the false positive signal reduces the likelihood of an accurate determination of the presence, absence and/or amount of the target nucleic acid sequence in the sample.

39. The method of any one of claims 1-38, wherein the presence of the one or more polymerase stoppers in the signal-generating oligonucleotide increases the likelihood of an accurate determination of the presence, absence and/or amount of the target nucleic acid sequence in the sample by at least about 1.1-fold as compared to an signal-generating oligonucleotide which does not comprise the one or more polymerase stoppers.

40. The method of any one of claims 1-39, wherein:
the generation of the first stalled extension product, the second stalled extension product, and/or third stalled extension product does not yield a false positive signal; and/or
the signal-generating oligonucleotide hybridizing the first stalled extension product, the second stalled extension product, and/or the third stalled extension product does not generate a false positive signal.

41. The method of any one of claims 1-40, wherein:
the nucleic acid amplification product reaches detectable levels at least about 1 minute, about 2 minutes, about 5 minutes, about 10 minutes, about 15 minutes, or about 20 minutes, before the first undesirable amplification product, the second undesirable amplification product, and/or the third undesirable amplification product reaches detectable levels; and/or
the signal reaches detectable levels at least about 1 minute, about 2 minutes, about 5 minutes, about 10 minutes, about 15 minutes, or about 20 minutes, before the false positive signal reaches detectable levels.

42. The method of any one of claims 1-41, wherein the appearance of detectable levels of the false positive signal, the first undesirable amplification product, the second undesirable amplification product, and/or the third undesirable amplification product is delayed by at least about 1 minute, about 2 minutes, about 5 minutes, about 10 minutes, about 15 minutes, or about 20 minutes, as compared to a comparable method wherein the signal-generating oligonucleotide which does not comprise the one or more polymerase stoppers.

43. The method of any one of claims 1-42, wherein the false positive signal, the first undesirable amplification product, the second undesirable amplification product, and/or the third undesirable amplification product does not reach detectable levels for at least about 5 minutes, about 10 minutes, about 15 minutes, or about 20 minutes, after the amplifying step begins.

44. The method of any one of claims 1-43, wherein the generation of the false positive signal, the first undesirable amplification product, the second undesirable amplification product, and/or the third undesirable amplification product is reduced by at least about 1.1-fold as compared to a comparable method wherein the signal-generating oligonucleotide which does not comprise the one or more polymerase stoppers.

45. The method of any one of claims 1-44, wherein amplifying the target nucleic acid sequence comprises generating the nucleic acid amplification product at detectable levels within about 20 minutes, about 15 minutes, or about 10 minutes; and/or
the detecting is performed in less than about 20 minutes, less than about 15 minutes, less than about 10 minutes, or less than about 5 minutes, from the time the reagent composition is contacted with the treated sample.

46. The method of any one of claims 1-45, wherein:
the lysis buffer comprises one or more of magnesium sulfate, ammonium sulfate, EDTA, and EGTA; and/or
the pH of the lysis buffer is about 1.0 to about 10.0, optionally the pH of the lysis buffer is about 2.2.

47. The method of any one of claims 1-46, wherein the sample nucleic acids comprise sample ribonucleic acids and/or sample deoxyribonucleic acids, optionally the sample nucleic acids comprise cellular RNA, mRNA, microRNA, bacterial RNA, viral RNA, or a combination thereof.

48. The method of any one of claims 1-47, wherein the one or more amplification reagents comprise:
a reverse transcriptase;
an enzyme having a hyperthermophile polymerase activity, optionally the enzyme having a hyperthermophile polymerase activity has a reverse transcriptase activity
a forward primer;
a reverse primer;
a reverse transcription primer; and/or
dNTPS.

49. The method of any one of claims 1-48, wherein the reagent composition is lyophilized, heat-dried, and/or comprises one or more additives, wherein the one or more additives comprise:
Tween 20, Triton X-100, and/or tween 80;
an amino acid;
a sugar or sugar alcohol, optionally the sugar or sugar alcohol comprises sucrose, lactose, trehalose, dextran, erythritol, arabitol, xylitol, sorbitol, mannitol, or any combination thereof; and/or
a polymer, optionally the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof,
optionally contacting the reagent composition with the treated sample comprises dissolving the reagent composition in the treated sample.

50. The method of any one of claims 1-49, wherein the one or more lytic reagents comprise:
about 0.001% (w/v) to about 1.0 (w/v) of the treated sample, optionally about 0.2% (w/v) of the treated sample; and/or
a detergent, optionally the detergent comprises one or more of a cationic surfactant, an anionic surfactant, a non-ionic surfactant, and an amphoteric surfactant.

51. The method of any one of claims 1-50, wherein the method:
is performed in a single reaction vessel;
does not comprise using any enzymes other than the reverse transcriptase and the enzyme having a hyperthermophile polymerase activity;
does not comprise using any enzyme other than the enzyme having a hyperthermophile polymerase activity;
does not comprise heat denaturing and/or enzymatic denaturing the nucleic acid during the amplification step; and/or
does not comprise contacting the nucleic acid with a single-stranded DNA binding protein.

52. The method of any one of claims 1-51, wherein:
the target nucleic acid sequence comprises a length of no longer than about 20 nucleotides to no longer than about 90 nucleotides, optionally the target nucleic acid sequence comprises a length of about 30 nucleotides;
the forward primer, the reverse primer, and/or the reverse transcription primer is about 8 to 16 bases long;
the nucleic acid amplification product is about 20 to 40 bases long; and/or
the spacer sequence comprises a portion of the target nucleic acid sequence, optionally the spacer sequence is 1 to 10 bases long.

53. The method of any one of claims 1-52, wherein:
the isothermal amplification condition comprises a constant temperature of about 30 °C to about 72 °C, further optionally about 55 °C to about 75 °C, optionally about 56 °C to about 67 °C;
the amplifying is performed (a) for a period of about 5 minutes to about 60 minutes, optionally the amplifying is performed for a period of about 15 minutes; and/or (b) in helicase-free, single-stranded binding protein-free, cleavage agent-free, and recombinase-free, isothermal amplification conditions;
the amplifying is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA), optionally the PCR is real-time PCR and/or quantitative real-time PCR (QRT-PCR);
the enzyme having a hyperthermophile polymerase activity has an amino acid sequence that is at least about 90% identical to the amino acid sequence of SEQ ID NO: 7 or a functional fragment thereof, optionally the enzyme having a hyperthermophile polymerase activity has an amino acid sequence that is at least about 95% identical to the amino acid sequence of SEQ ID NO: 7, further optionally the enzyme having a hyperthermophile polymerase activity is a polymerase comprising the amino acid sequence of SEQ ID NO: 7, optionally the enzyme having a hyperthermophile polymerase activity has low or no exonuclease activity; and/or
the sample ribonucleic acids are contacted with the reverse transcriptase and the enzyme having a hyperthermophile polymerase activity simultaneously, optionally the sample ribonucleic acids are contacted with the reverse transcriptase, the enzyme having a hyperthermophile polymerase activity, and the forward and reverse primers simultaneously, further optionally the sample ribonucleic acids are contacted with the reverse transcriptase, the enzyme having a hyperthermophile polymerase activity, the forward primer, the reverse primer, and the reverse transcription primer simultaneously.

54. The method of any one of claims 1-53, wherein:
the biological entities comprise one or more of prokaryotic cells, eukaryotic cells, viral particles, exosomes, protoplasts, and microvesicles;
the biological entities comprise a virus, a bacteria, a fungi, a protozoa, portions thereof, or any combination thereof; and/or
the target nucleic acid sequence is a nucleic acid sequence of a virus, bacteria, fungi, or protozoa, optionally the sample nucleic acids are derived from a virus, bacteria, fungi, or protozoa.

55. The method of any one of claims 1-54, wherein:
the virus is SARS-CoV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), or Hantavirus;
the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes;
the fungi comprises one or more of Cryptococcus neoformans, Pneumocystis carinii, Histoplasma capsulatum, Blastomyces dermatitidis, Coccidioides immitis, and Trichophyton rubrum; and/or
the protozoa comprises one or more of Trypanosoma cruzi, Leishmania sp., Plasmodium, Entamoeba histolytica, Babesia microti, Giardia lamblia, Cyclospora sp., and Eimeria sp.

56. The method of any one of claims 1-55, wherein the sample is a biological sample or an environmental sample,
wherein the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof; and/or
wherein the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

57. The method of any one of claims 1-56, wherein:
the amplifying step comprises multiplex amplification of two or more target nucleic acid sequences, and wherein the detecting step comprises multiplex detection of two or more nucleic acid amplification products derived from said two or more target nucleic acid sequences, optionally the two or more target nucleic acid sequences are specific to two or more different organisms, further optionally the two or more different organisms comprise one or more of SARS-CoV-2, Influenza A, Influenza B, and/or Influenza C;
the amplifying does not comprise one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA), optionally the amplifying does not comprise LAMP;
the amplifying comprises one or more of the following: APA, LAMP, HDA, RPA, SDA, NASBA, TMA, NEAR, RCA, MDA, RAM, cHDA, SPIA, SMART, 3SR, GEAR and IMDA, optionally the amplifying does not comprise LAMP; and/or
the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

58. A signal-generating oligonucleotide capable of hybridizing to a nucleic acid amplification product, comprising
a 5′ subdomain, a 3′ subdomain, an intramolecular nucleotide base pairing between the 5′ subdomain and the 3′ subdomain, and a loop domain between the 5′ subdomain and the 3′ subdomain,
wherein the intramolecular nucleotide base pairing between the 5′ subdomain and the 3′ subdomain are capable of forming a paired stem domain,
wherein the loop domain comprises one or more polymerase stoppers, and
wherein the 5′ subdomain, the paired stem domain, and/or the 3′ subdomain does not comprise the one or more polymerase stoppers.

59. The signal-generating oligonucleotide of claim 58, wherein the nucleic acid amplification product is generated by amplifying a target nucleic acid sequence comprising a first strand and a second strand complementary to each other.

60. The signal-generating oligonucleotide of any one of claims 58-59, wherein the nucleic acid amplification product comprises:
(1) the sequence of a forward primer, and the reverse complement thereof,
(2) the sequence of a reverse primer, and the reverse complement thereof, and
(3) a spacer sequence flanked by (1) the sequence of the forward primer and the reverse complement thereof and (2) the sequence of the reverse primer and the reverse complement thereof, wherein the spacer sequence is 1 to 10 bases long.

61. The signal-generating oligonucleotide of any one of claims 58-60, wherein:
the signal-generating oligonucleotide comprises a first region comprising the sequence of at least a portion of the reverse primer;
the signal-generating oligonucleotide comprises a second region comprising a sequence complementary to at least a portion of the forward primer; and/or
the signal-generating oligonucleotide comprises a spacer region comprising the sequence of at least a portion of the spacer sequence,
optionally, the first region, the second region, and/or the spacer region comprises one or more polymerase stoppers.

62. The signal-generating oligonucleotide of any one of claims 58-61, wherein:
the first region comprises a sequence complementary to at least two 3′ terminal nucleotides of the forward primer and/or the reverse primer;
the second region comprises a sequence complementary to at least two 3′ terminal nucleotides of the forward primer and/or the reverse primer; and/or
the spacer region comprises a sequence complementary to at least two 3′ terminal nucleotides of the forward primer and/or the reverse primer

63. The signal-generating oligonucleotide of any one of claims 58-62, wherein:
the forward primer is capable of hybridizing to a sequence of the first strand of the target nucleic acid sequence, and
the reverse primer is capable of hybridizing to a sequence of the second strand of the target nucleic acid sequence,
optionally the nucleic acid amplification product is generated by amplifying the target nucleic acid sequence with the forward primer and the reverse primer.

64. The signal-generating oligonucleotide of any one of claims 58-63, wherein:
the signal-generating oligonucleotide is about 10 nucleotides to about 100 nucleotides in length;
the forward primer and/or reverse primer is about 5 nucleotides to about 25 nucleotides in length;
the second region, the spacer region, and/or the first region is about 1 nucleotide to about 25 nucleotides in length; and/or
the 5′ subdomain, the 3′ subdomain, the loop domain, the 5′ terminal domain, and/or the 3′ terminal domain is about 1 nucleotide to about 25 nucleotides in length.

65. The signal-generating oligonucleotide of any one of claims 58-64, wherein:
the signal-generating oligonucleotide comprises a 5′ terminal domain situated 5′ of the 5′ subdomain, and/or
the signal-generating oligonucleotide comprises a 3′ terminal domain situated 3′ of the 5′ subdomain,
optionally the 5′ terminal domain and/or the 3′ terminal domain does not comprise the one or more polymerase stoppers.

66. The signal-generating oligonucleotide of any one of claims 58-65, wherein:
the first region comprises at least a portion of the 5′ subdomain and/or loop domain,
the spacer region comprises at least a portion of the loop domain, and
the second region comprises at least a portion of the loop domain and/or 3′ subdomain.

67. The signal-generating oligonucleotide of any one of claims 58-66, wherein the one or more polymerase stoppers comprise one or more 2′-O-methyl (2′OM) RNA nucleotides.

68. The signal-generating oligonucleotide of any one of claims 58-67, wherein the one or more polymerase stoppers comprise one or more of an abasic site, a stable abasic site, a chemically trapped abasic site, or any combination thereof.

69. The signal-generating oligonucleotide of claim 68, wherein:
the stable abasic site comprises 1′,2′-dideoxy;
the chemically trapped abasic site comprises an abasic site reacted with alkoxy amine or sodium borohydride;
the abasic site comprises an apurinic site, an apyrimidinic site, or both; and/or
the abasic site is generated by an alkylating agent or an oxidizing agent.

70. The signal-generating oligonucleotide of any one of claims 58-69, wherein the one or polymerase stoppers comprise:
one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-m ethyl cytidines, one or more 5-hydroxymethylcytidines, one or more 2′-O-Methyl RNA bases, one or more unmethylated RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(o)NHCH₂—C₆H₃NO₂—CH(CH₃)OPO₃] groups, one or more hexandiol groups, one or more spacer 9 (iSp9) [(OCH₂CH₂)₃OPO₃] groups, one or more spacer 18 (iSp18) [(OCH₂CH₂₆OPO₃] groups, or any combination thereof.

71. The signal-generating oligonucleotide of any one of claims 58-70, wherein:
the signal-generating oligonucleotide comprises one or more phosphorothioate linkages and/or one or more locked nucleic acids;
the signal-generating oligonucleotide comprises a TaqMan detection probe oligonucleotide, a molecular beacon detection probe oligonucleotide, or a molecular torch detection probe oligonucleotide;
the signal-generating oligonucleotide comprises a label, optionally the label comprises a quenchable label, further optionally the quenchable label is a fluorophore;
the signal-generating oligonucleotide comprises a quencher, optionally (a) the label is situated in the 3′ terminal domain and the quencher is situated in the 5′ terminal domain, and/or (b) the label is situated in the 5′ terminal domain and the quencher is situated in the 3′ terminal domain; and/or
the label is capable of generating a signal upon the signal-generating oligonucleotide hybridizing the nucleic acid amplification product, optionally the signal is fluorescence.

72. The signal-generating oligonucleotide of any one of claims 58-71,
wherein, upon the forward primer binding the signal-generating oligonucleotide to form a first undesirable duplex, the one or more polymerase stoppers are capable of stopping polymerase extension of the forward primer of the first undesirable duplex to the 5′ end of the signal-generating oligonucleotide,
optionally, the one or more polymerase stoppers are capable of stopping polymerase extension of the forward primer of the first undesirable duplex beyond the one or more polymerase stoppers of the signal-generating oligonucleotide.

73. The signal-generating oligonucleotide of any one of claims 58-72,
wherein, upon the reverse primer binding the signal-generating oligonucleotide to form a second undesirable duplex, the one or more polymerase stoppers are capable of stopping polymerase extension of the reverse primer of the second undesirable duplex to the 5′ end of the signal-generating oligonucleotide,
optionally, the one or more polymerase stoppers are capable of stopping polymerase extension of the reverse primer of the second undesirable duplex beyond the one or more polymerase stoppers of the signal-generating oligonucleotide.

74. The signal-generating oligonucleotide of any one of claims 58-73,
wherein, upon an extraneous nucleic acid binding the signal-generating oligonucleotide to form a third undesirable duplex, the one or more polymerase stoppers are capable of stopping polymerase extension of the extraneous nucleic acid of the third undesirable duplex to the 5′ end of the signal-generating oligonucleotide,
optionally, the one or more polymerase stoppers are capable of stopping polymerase extension of the extraneous nucleic acid of the third undesirable duplex beyond the one or more polymerase stoppers of the signal-generating oligonucleotide.

75. A kit for detecting a target nucleic acid sequence in a sample, the kit comprising:
(a) the signal-generating oligonucleotide of any one of claims 1-74;
(b) a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent, and wherein the detergent comprises one or more of a cationic surfactant, an anionic surfactant, a non-ionic surfactant, and an amphoteric surfactant; and/or
(c) a reagent composition comprising one or more amplification reagents comprising one or more components for amplifying the target nucleic acid sequence under isothermal amplification conditions, wherein said one or more components for amplifying comprise:
(i) a forward primer and a reverse primer, wherein the forward primer is capable of hybridizing to a sequence of a first strand of the target nucleic acid sequence, and the reverse primer is capable of hybridizing to a sequence of a second strand of the target nucleic acid sequence; and/or
(ii) an enzyme having a hyperthermophile polymerase activity capable of generating a nucleic acid amplification product, optionally the enzyme having a hyperthermophile polymerase activity has an amino acid sequence that is at least about 90% identical to the amino acid sequence of SEQ ID NO: 7 or a functional fragment thereof, optionally the enzyme having a hyperthermophile polymerase activity has an amino acid sequence that is at least about 95% identical to the amino acid sequence of SEQ ID NO: 7, further optionally the enzyme having a hyperthermophile polymerase activity is a polymerase comprising the amino acid sequence of SEQ ID NO: 7.

76. The kit of claim 75, wherein the reagent composition comprises a reverse transcriptase and/or a reverse transcription primer.

77. The kit of any one of claims 75-76, wherein the nucleic acid amplification product is about 20 to 40 bases long, and wherein the nucleic acid amplification product comprises:
(1) the sequence of the forward primer, and the reverse complement thereof,
(2) the sequence of the reverse primer, and the reverse complement thereof, and
(3) a spacer sequence flanked by (1) the sequence of the forward primer and the reverse complement thereof and (2) the sequence of the reverse primer and the reverse complement thereof, wherein the spacer sequence is 1 to 10 bases long.

78. The kit of any one of claims 75-77, wherein:
the biological entities comprise one or more of prokaryotic cells, eukaryotic cells, viral particles, exosomes, protoplasts, and microvesicles;
the biological entities comprise a virus, a bacteria, a fungi, a protozoa, portions thereof, or any combination thereof; and/or
the target nucleic acid sequence is a nucleic acid sequence of a virus, bacteria, fungi, or protozoa, optionally the sample nucleic acids are derived from a virus, bacteria, fungi, or protozoa.

79. The kit of any one of claims 75-78,
wherein the virus is SARS-CoV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), and Hantavirus;
wherein the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes;
wherein the fungi comprises one or more of Cryptococcus neoformans, Pneumocystis carinii, Histoplasma capsulatum, Blastomyces dermatitidis, Coccidioides immitis, and Trichophyton rubrum; and/or
wherein the protozoa comprises one or more of Trypanosoma cruzi, Leishmania sp., Plasmodium, Entamoeba histolytica, Babesia microti, Giardia lamblia, Cyclospora sp., and Eimeria sp.

80. The kit of any one of claims 75-79, wherein the reagent composition is lyophilized and/or heat-dried and comprises one or more additives, wherein the one or more additives comprise:
an amino acid;
a sugar or sugar alcohol, optionally the sugar or sugar alcohol comprises sucrose, lactose, trehalose, dextran, erythritol, arabitol, xylitol, sorbitol, mannitol, or any combination thereof; and/or
a polymer, optionally the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

81. A method of detecting N. Gonorrhea in a sample, comprising:
contacting said sample with at least one pair of primers,
wherein said at least one pair of primers is capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity to any one of the sequences of SEQ ID NOs: 2-3 and 16-17;
generating amplicons of the target nucleic acid sequence, if said sample comprises N. Gonorrhea; and
determining the presence or amount of said amplicons as an indication of the presence of N. Gonorrhea in said sample.

82. The method of claim 81, wherein the at least one pair of primers comprises a first primer comprising the sequence of SEQ ID NOs: 2 or 17, and a second primer comprising the sequence of SEQ ID NOs: 3 or 16.

83. The method of any one of claims 81-82, wherein:
the at least one pair of primers capable of hybridizing to the target nucleic acid sequence of N. Gonorrhea is SEQ ID NOs: 2 and 3, SEQ ID NOs: 2 and 16, SEQ ID NOs: 17 and 3, or SEQ ID NOs: 17 and 16.

84. The method of any one of claims 81-83, wherein determining the presence or amount of amplicons of the target nucleic acid sequence comprises contacting the amplicons with one or more the signal-generating oligonucleotides, wherein each of the one or more the signal-generating oligonucleotides comprises a sequence selected from the group consisting of SEQ ID NOs: 1 and 12-14, or a sequence that exhibits at least about 85% identity to a sequence selected from the group consisting of SEQ ID NOs: 1 and 12-14, optionally wherein:
each of the one or more the signal-generating oligonucleotides comprises a sequence selected from the group consisting of SEQ ID NOs: 1 and 12-14; and/or
each of the one or more the signal-generating oligonucleotides consists of a sequence selected from the group consisting of SEQ ID NOs: 1 and 12-14.

85. A method of detecting C. trachomatis in a sample, comprising:
contacting said sample with at least one pair of primers,
wherein said at least one pair of primers is capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity to any one of the sequences of SEQ ID NOs: 20-23, 25-26, 28-29, and 31;
generating amplicons of the target nucleic acid sequence, if said sample comprises C. trachomatis; and
determining the presence or amount of said amplicons as an indication of the presence of C. trachomatis in said sample.

86. The method of claim 85, wherein the at least one pair of primers comprises a first primer comprising the sequence of SEQ ID NOs: 20, 22, 25, or 28, and a second primer comprising the sequence of SEQ ID NOs: 21, 23, 26, 29, or 31.

87. The method of any one of claims 85-86, wherein:
the at least one pair of primers capable of hybridizing to the target nucleic acid sequence of C. trachomatis is SEQ ID NOs: 20 and 21, SEQ ID NOs: 20 and 23, SEQ ID NOs: 20 and 26, SEQ ID NOs: 20 and 29, SEQ ID NOs: 20 and 31, SEQ ID NOs: 22 and 21, SEQ ID NOs: 22 and 23, SEQ ID NOs: 22 and 26, SEQ ID NOs: 22 and 29, SEQ ID NOs: 22 and 31, SEQ ID NOs: 25 and 21, SEQ ID NOs: 25 and 23, SEQ ID NOs: 25 and 26, SEQ ID NOs: 25 and 29, SEQ ID NOs: 25 and 31, SEQ ID NOs: 28 and 21, SEQ ID NOs: 28 and 23, SEQ ID NOs: 28 and 26, SEQ ID NOs: 28 and 29, or SEQ ID NOs: 28 and 31.

88. The method of any one of claims 85-87, wherein determining the presence or amount of amplicons of the target nucleic acid sequence comprises contacting the amplicons with one or more the signal-generating oligonucleotides, wherein each of the one or more the signal-generating oligonucleotides comprises a sequence selected from the group consisting of SEQ ID NOs: 15, 18-19, 24, 27, 30, and 32, or a sequence that exhibits at least about 85% identity to a sequence selected from the group consisting of SEQ ID NOs: 15, 18-19, 24, 27, 30, and 32, optionally wherein:
each of the one or more the signal-generating oligonucleotides comprises a sequence selected from the group consisting of SEQ ID NOs: 15, 18-19, 24, 27, 30, and 32; and/or
each of the one or more the signal-generating oligonucleotides consists of a sequence selected from the group consisting of SEQ ID NOs: 15, 18-19, 24, 27, 30, and 32.

89. A method of detecting an Influenza B virus in a sample, comprising:
contacting said sample with at least one pair of primers,
wherein said at least one pair of primers is capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity to any one of the sequences of SEQ ID NOs: 36-37 and 41;
generating amplicons of the target nucleic acid sequence, if said sample comprises an Influenza B virus; and
determining the presence or amount of said amplicons as an indication of the presence of an Influenza B virus in said sample.

90. The method of claim 89, wherein the at least one pair of primers comprises a first primer comprising the sequence of SEQ ID NO: 36, and a second primer comprising the sequence of SEQ ID NOs: 37 or 41.

91. The method of any one of claims 89-90, wherein:
the at least one pair of primers capable of hybridizing to the target nucleic acid sequence of an Influenza B virus is SEQ ID NOs: 36 and 37, or SEQ ID NOs: 36 and 41.

92. The method of any one of claims 89-91, wherein determining the presence or amount of amplicons of the target nucleic acid sequence comprises contacting the amplicons with one or more the signal-generating oligonucleotides, wherein each of the one or more the signal-generating oligonucleotides comprises a sequence selected from the group consisting of SEQ ID NOs: 39-40 and 42-45, or a sequence that exhibits at least about 85% identity to a sequence selected from the group consisting of SEQ ID NOs: 39-40 and 42-45, optionally wherein:
each of the one or more the signal-generating oligonucleotides comprises a sequence selected from the group consisting of SEQ ID NOs: 39-40 and 42-45; and/or
each of the one or more the signal-generating oligonucleotides consists of a sequence selected from the group consisting of SEQ ID NOs: 39-40 and 42-45.

93. The method of any one of claims 89-92, comprising contacting sample ribonucleic acids of the sample with a reverse transcriptase and a primer exhibiting at least about 85% identity to SEQ ID NO: 38.

94. The method of any one of claims 81-93, wherein the sample is a biological sample or an environmental sample,
wherein the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof; and/or
wherein the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

95. The method of any one of claims 81-94, wherein the sample is contacted with a reagent composition comprising the at least one pair of primers to generate the amplification reaction mixture.

96. The method of any one of claims 81-95, comprising:
contacting the sample comprising biological entities with a lysis buffer to generate a treated sample, wherein the lysis buffer comprises one or more lytic agents capable of lysing biological entities to release sample nucleic acids comprised therein, and wherein the sample nucleic acids are suspected of comprising the target nucleic acid sequence; and
contacting a reagent composition comprising the at least one pair of primers with the treated sample to generate the amplification reaction mixture, wherein the reagent composition comprises one or more amplification reagents.

97. The method of any one of claims 81-96, wherein the one or more amplification reagents comprise:
a reverse transcriptase;
an enzyme having a hyperthermophile polymerase activity, optionally the enzyme having a hyperthermophile polymerase activity has a reverse transcriptase activity;
a reverse transcription primer; and/or
dNTPS.

98. The method of any one of claims 81-97, wherein generating amplicons of the target nucleic acid sequence comprises:
amplifying the target nucleic acid sequence in the amplification reaction mixture under an amplification condition, thereby generating amplicons of the target nucleic acid sequence.

99. The method of claim 98, wherein:
the amplifying is carried out using a method selected from the group consisting of polymerase chain reaction (PCR), ligase chain reaction (LCR), loop-mediated isothermal amplification (LAMP), strand displacement amplification (SDA), replicase-mediated amplification, Immuno-amplification, nucleic acid sequence based amplification (NASBA), self-sustained sequence replication (3SR), rolling circle amplification, and transcription-mediated amplification (TMA), optionally the PCR is real-time PCR and/or quantitative real-time PCR (QRT-PCR);
the amplifying does not comprise one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA), optionally the amplifying does not comprise LAMP;
the amplifying comprises one or more of the following: APA, LAMP, HDA, RPA, SDA, NASBA, TMA, NEAR, RCA, MDA, RAM, cHDA, SPIA, SMART, 3SR, GEAR and IMDA, optionally the amplifying does not comprise LAMP; and/or
the method does not comprise one or more of the following: (i) dilution of the treated sample; (ii) dilution of the amplification reaction mixture; (iii) heat denaturation of the treated sample; (iv) sonication of the treated sample; (v) sonication of the amplification reaction mixture; (vi) the addition of ribonuclease inhibitors to the treated sample; (vii) the addition of ribonuclease inhibitors to the amplification reaction mixture; (viii) purification of the sample; (ix) purification of the sample nucleic acids; (x) purification of the nucleic acid amplification product; (xi) removal of the one or more lytic agents from the treated sample or the amplification reaction mixture; (xii) heat denaturing and/or enzymatic denaturing of the sample nucleic acids prior to and/or during amplification; and (xiii) the addition of ribonuclease H to the treated sample or amplification reaction mixture.

100. The method of any one of claims 81-99, wherein the determining step comprises contacting the amplicons of the target nucleic acid sequence with the signal-generating oligonucleotide for hybridization, optionally the determining comprises use of a real-time detection method.

101. The method of any one of claims 81-100, wherein:
a label of the signal-generating oligonucleotide is capable of generating a signal upon the signal-generating oligonucleotide hybridizing the amplicons of the target nucleic acid sequence; and/or
upon the signal-generating oligonucleotide hybridizing the amplicons of the target nucleic acid sequence, the label generates a signal, optionally the signal is fluorescence.

102. A composition for the detection of N. Gonorrhea in a sample, comprising:
at least one pair of primers capable of hybridizing to a target nucleic acid sequence of N. Gonorrhea, wherein each primer in said at least one pair of primers comprises any one of the sequences of SEQ ID NOs: 2-3 and 16-17, or a sequence that exhibits at least about 85% identity to any one of the sequences of SEQ ID NOs: 2-3 and 16-17.

103. The composition of claim 102, wherein:
the at least one pair of primers capable of hybridizing to the target nucleic acid sequence of N. Gonorrhea comprises a primer comprising the sequence of SEQ ID NOs: 2 or 17 and a primer comprising the sequence of SEQ ID NOs: 3 or 16.

104. The composition of any one of claims 102-103, further comprising one or more the signal-generating oligonucleotides, wherein each of the one or more the signal-generating oligonucleotides comprises a sequence selected from the group consisting of SEQ ID NOs: 1 and 12-14, or a sequence that exhibits at least about 85% identity to a sequence selected from the group consisting of SEQ ID NOs: 1 and 12-14.

105. The composition of any one of claims 102-104, wherein each of the one or more the signal-generating oligonucleotides comprises a sequence selected from the group consisting of SEQ ID NOs: 1 and 12-14.

106. The composition of any one of claims 102-105, wherein each of the one or more the signal-generating oligonucleotides consists of a sequence selected from the group consisting of SEQ ID NOs: 1 and 12-14.

107. A composition for the detection of C. trachomatis in a sample, comprising:
at least one pair of primers capable of hybridizing to a target nucleic acid sequence of C. trachomatis, wherein each primer in said at least one pair of primers comprises any one of the sequences of SEQ ID NOs: 20-23, 25-26, 28-29, and 31, or a sequence that exhibits at least about 85% identity to any one of the sequences of SEQ ID NOs: 20-23, 25-26, 28-29, and 31.

108. The composition of claim 107, wherein:
the at least one pair of primers capable of hybridizing to the target nucleic acid sequence of C. trachomatis comprises a primer comprising the sequence of SEQ ID NOs: 20, 22, 25, or 28 and a primer comprising the sequence of SEQ ID NOs: 21, 23, 26, 29, or 31.

109. The composition of any one of claims 107-108, further comprising one or more the signal-generating oligonucleotides, wherein each of the one or more the signal-generating oligonucleotides comprises a sequence selected from the group consisting of SEQ ID NOs: 15, 18-19, 24, 27, 30, and 32, or a sequence that exhibits at least about 85% identity to a sequence selected from the group consisting of SEQ ID NOs: 15, 18-19, 24, 27, 30, and 32.

110. The composition of any one of claims 107-109, wherein each of the one or more the signal-generating oligonucleotides comprises a sequence selected from the group consisting of SEQ ID NOs: 15, 18-19, 24, 27, 30, and 32.

111. The composition of any one of claims 107-110, wherein each of the one or more the signal-generating oligonucleotides consists of a sequence selected from the group consisting of SEQ ID NOs: 15, 18-19, 24, 27, 30, and 32.

112. A composition for the detection of an Influenza B virus in a sample, comprising:
at least one pair of primers capable of hybridizing to a target nucleic acid sequence of an Influenza B virus, wherein each primer in said at least one pair of primers comprises any one of the sequences of SEQ ID NOs: 36-37 and 41, or a sequence that exhibits at least about 85% identity to any one of the sequences of SEQ ID NOs: 36-37 and 41.

113. The composition of claim 112, wherein:
the at least one pair of primers capable of hybridizing to the target nucleic acid sequence of an Influenza B virus comprises a primer comprising the sequence of SEQ ID NO: 36 and a primer comprising the sequence of SEQ ID NOs: 37 or 41.

114. The composition of any one of claims 112-113, further comprising a primer exhibiting at least about 85% identity to SEQ ID NO: 38.

115. The composition of any one of claims 112-114, further comprising one or more the signal-generating oligonucleotides, wherein each of the one or more the signal-generating oligonucleotides comprises a sequence selected from the group consisting of SEQ ID NOs: 39-40 and 42-45, or a sequence that exhibits at least about 85% identity to a sequence selected from the group consisting of SEQ ID NOs: 39-40 and 42-45.

116. The composition of any one of claims 112-115, wherein each of the one or more the signal-generating oligonucleotides comprises a sequence selected from the group consisting of SEQ ID NOs: 39-40 and 42-45.

117. The composition of any one of claims 112-116, wherein each of the one or more the signal-generating oligonucleotides consists of a sequence selected from the group consisting of SEQ ID NOs: 39-40 and 42-45.

118. The method or composition of any one of claims 81-117,
wherein the signal-generating oligonucleotide comprises a 5′ subdomain and a 3′ subdomain,
wherein the signal-generating oligonucleotide comprises a loop domain situated between the 5′ subdomain and the 3′ subdomain,
wherein intramolecular nucleotide base pairing between the 5′ subdomain and the 3′ subdomain are capable of forming a paired stem domain,
wherein the loop domain comprises one or more polymerase stoppers, and
wherein the 5′ subdomain, the paired stem domain, and/or the 3′ subdomain does not comprise the one or more polymerase stoppers.

119. The method or composition of claim 118, wherein:
the signal-generating oligonucleotide comprises a 5′ terminal domain situated 5′ of the 5′ subdomain, and/or
the signal-generating oligonucleotide comprises a 3′ terminal domain situated 3′ of the 5′ subdomain,
optionally the 5′ terminal domain and/or the 3′ terminal domain does not comprise the one or more polymerase stoppers.

120. The method or composition of any one of claims 118-119, wherein the one or more polymerase stoppers comprise one or more 2′-O-methyl (2′OM) RNA nucleotides.

121. The method or composition of any one of claims 118-120, wherein the one or more polymerase stoppers comprise one or more of an abasic site, a stable abasic site, a chemically trapped abasic site, or any combination thereof.

122. The method or composition of any one of claims 118-121, wherein:
the stable abasic site comprises 1′,2′-dideoxy;
the chemically trapped abasic site comprises an abasic site reacted with alkoxy amine or sodium borohydride;
the abasic site comprises an apurinic site, an apyrimidinic site, or both; and/or
the abasic site is generated by an alkylating agent or an oxidizing agent.

123. The method or composition of any one of claims 118-122, wherein:
the one or polymerase stoppers comprise: one or more nitroindoles, one or more inosines, one or more acridines, one or more 2-aminopurines, one or more 2-6-diaminopurines, one or more 5-bromo-deoxyuridines, one or more inverted thymidines (inverted dTs), one or more inverted dideoxy-thymidines (ddTs), one or more dideoxy-cytidines (ddCs), one or more 5-methyl cytidines, one or more 5-hydroxymethylcytidines, one or more 2′-O-Methyl RNA bases, one or more unmethylated RNA bases, one or more Iso-deoxycytidines (Iso-dCs), one or more Iso-deoxyguanosines (Iso-dGs), one or more C3 (OC₃H₆OPO₃) groups, one or more photo-cleavable (PC) [OC₃H₆—C(o)NHCH₂—C₆H₃NO₂—CH(CH₃)OPO₃] groups, one or more hexandiol groups, one or more spacer 9 (iSp9) [(OCH₂CH₂)₃OPO₃] groups, one or more spacer 18 (iSp18) [(OCH₂CH₂₆OPO₃] groups, or any combination thereof, the signal-generating oligonucleotide comprises one or more phosphorothioate linkages and/or one or more locked nucleic acids;
the signal-generating oligonucleotide comprises a TaqMan detection probe oligonucleotide, a molecular beacon detection probe oligonucleotide, or a molecular torch detection probe oligonucleotide; and/or
the signal-generating oligonucleotide comprises a label, optionally the label comprises a quenchable label, further optionally the quenchable label is a fluorophore.

124. A signal-generating oligonucleotide or primer up to about 100 nucleotides in length which is capable of hybridizing to a target nucleic acid sequence of N. Gonorrhea, wherein said signal-generating oligonucleotide or primer comprises a sequence selected from the group consisting of SEQ ID NOs: 1-3, 12-14, and 16-17, or sequence that exhibits at least about 85% identity to a sequence selected from the group consisting of SEQ ID NOs: 1-3, 12-14, and 16-17, optionally wherein:
said signal-generating oligonucleotide or primer consists of a sequence selected from the group consisting of SEQ ID NOs: 1-3, 12-14, and 16-17, or sequence that exhibits at least about 85% identity to a sequence selected from the group consisting of SEQ ID NOs: 1-3, 12-14, and 16-17;
said signal-generating oligonucleotide or primer comprises a sequence selected from the group consisting of SEQ ID NOs: 1-3, 12-14, and 16-17; and/or
said signal-generating oligonucleotide or primer consists of a sequence selected from the group consisting of SEQ ID NOs: 1-3, 12-14, and 16-17.

125. A signal-generating oligonucleotide or primer up to about 100 nucleotides in length which is capable of hybridizing to a target nucleic acid sequence of C. trachomatis, wherein said signal-generating oligonucleotide or primer comprises a sequence selected from the group consisting of SEQ ID NOs: 15 and 18-32, or sequence that exhibits at least about 85% identity to a sequence selected from the group consisting of SEQ ID NOs: 15 and 18-32, optionally wherein:
said signal-generating oligonucleotide or primer consists of a sequence selected from the group consisting of SEQ ID NOs: 15 and 18-32, or sequence that exhibits at least about 85% identity to a sequence selected from the group consisting of SEQ ID NOs: 15 and 18-32;
said signal-generating oligonucleotide or primer comprises a sequence selected from the group consisting of SEQ ID NOs: 15 and 18-32; and/or
said signal-generating oligonucleotide or primer consists of a sequence selected from the group consisting of SEQ ID NOs: 15 and 18-32.

126. A signal-generating oligonucleotide or primer up to about 100 nucleotides in length which is capable of hybridizing to a target nucleic acid sequence of an Influenza B virus, wherein said signal-generating oligonucleotide or primer comprises a sequence selected from the group consisting of SEQ ID NOs: 36-45, or sequence that exhibits at least about 85% identity to a sequence selected from the group consisting of SEQ ID NOs: 36-45, optionally wherein:
said signal-generating oligonucleotide or primer consists of a sequence selected from the group consisting of SEQ ID NOs: 36-45, or sequence that exhibits at least about 85% identity to a sequence selected from the group consisting of SEQ ID NOs: 36-45;
said signal-generating oligonucleotide or primer comprises a sequence selected from the group consisting of SEQ ID NOs: 36-45; and/or
said signal-generating oligonucleotide or primer consists of a sequence selected from the group consisting of SEQ ID NOs: 36-45.

127. A signal-generating oligonucleotide or primer up to about 100 nucleotides in length which is capable of hybridizing to a target nucleic acid sequence of an Influenza A virus, wherein said signal-generating oligonucleotide or primer comprises a sequence selected from the group consisting of SEQ ID NOs: 33-35, or sequence that exhibits at least about 85% identity to a sequence selected from the group consisting of SEQ ID NOs: 33-35, optionally wherein:
said signal-generating oligonucleotide or primer consists of a sequence selected from the group consisting of SEQ ID NOs: 33-35, or sequence that exhibits at least about 85% identity to a sequence selected from the group consisting of SEQ ID NOs: 33-35;
said signal-generating oligonucleotide or primer comprises a sequence selected from the group consisting of SEQ ID NOs: 33-35; and/or
said signal-generating oligonucleotide or primer consists of a sequence selected from the group consisting of SEQ ID NOs: 33-35.

128. A composition, comprising two or more of the signal-generating oligonucleotide or primer of any one of claims 124-127,

129. The composition of claim 128, comprising:
a lysis buffer comprising one or more lytic agents capable of lysing biological entities to release sample nucleic acids comprised therein, wherein the sample nucleic acids are suspected of comprising a target nucleic acid sequence, optionally the one or more lytic agents comprise a detergent, and wherein the detergent comprises one or more of a cationic surfactant, an anionic surfactant, a non-ionic surfactant, and an amphoteric surfactant; and/or
a reagent composition comprising one or more amplification reagents comprising one or more components for amplifying the target nucleic acid sequence under isothermal amplification conditions.

130. The composition of any one of claims 128-129, wherein said one or more components for amplifying comprise:
an enzyme having a hyperthermophile polymerase activity capable of generating a nucleic acid amplification product, optionally the enzyme having a hyperthermophile polymerase activity has an amino acid sequence that is at least about 90% identical to the amino acid sequence of SEQ ID NO: 7 or a functional fragment thereof, optionally the enzyme having a hyperthermophile polymerase activity has an amino acid sequence that is at least about 95% identical to the amino acid sequence of SEQ ID NO: 7, further optionally the enzyme having a hyperthermophile polymerase activity is a polymerase comprising the amino acid sequence of SEQ ID NO: 7.


SEQUENCE LISTING (45 sequences)

SEQ ID NO: 1 - DNA, 23 bp, synthetic construct
  source 1..23: mol_type = other DNA
acagacgaagcggatacctcgtc

SEQ ID NO: 2 - DNA, 13 bp, synthetic construct
  source 1..13: mol_type = other DNA
gaaattccaaaga

SEQ ID NO: 3 - DNA, 10 bp, synthetic construct
  source 1..10: mol_type = other DNA
acgaagcgga

SEQ ID NO: 4 - DNA, 27 bp, synthetic construct
  source 1..27: mol_type = other DNA
gaaattccaaagaggtatccgcttcgt

SEQ ID NO: 5 - DNA, 27 bp, synthetic construct
  source 1..27: mol_type = other DNA
acgaagcggatacctctttggaatttc

SEQ ID NO: 6 - DNA, 28 bp, synthetic construct
  source 1..28: mol_type = other DNA
acgaagcggaggtatccgcttcgtctgt

SEQ ID NO: 7 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 8 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIDTLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 9 - DNA, 12 bp, synthetic construct
  source 1..12: mol_type = other DNA
atgcatgcatgc

SEQ ID NO: 10 - DNA, 12 bp, synthetic construct
  source 1..12: mol_type = other DNA
gcatgcatgcat

SEQ ID NO: 11 - DNA, 18 bp, synthetic construct
  source 1..18: mol_type = other DNA
atgcataaaaaagcatgc

SEQ ID NO: 12 - DNA, 23 bp, synthetic construct
  source 1..23: mol_type = other DNA
  modified_base 7: mod_base = OTHER; note = locked nucleic acid base
  modified_base 8: mod_base = OTHER; note = locked nucleic acid base
  modified_base 16: mod_base = OTHER; note = locked nucleic acid base
  modified_base 18: mod_base = OTHER; note = locked nucleic acid base
  modified_base 19: mod_base = OTHER; note = locked nucleic acid base
  modified_base 23: mod_base = OTHER; note = locked nucleic acid base
acagacgaagcggatacctcgtc

SEQ ID NO: 13 - DNA, 23 bp, synthetic construct
  source 1..23: mol_type = other DNA
  modified_base 7: mod_base = OTHER; note = locked nucleic acid base
  modified_base 8: mod_base = OTHER; note = locked nucleic acid base
  modified_base 16: mod_base = OTHER; note = locked nucleic acid base
  modified_base 18: mod_base = OTHER; note = locked nucleic acid base
  modified_base 19: mod_base = OTHER; note = locked nucleic acid base
  modified_base 23: mod_base = OTHER; note = locked nucleic acid base
  modified_base 14: mod_base = OTHER; note = 2'-O-methyladenosine
acagacgaagcggatacctcgtc

SEQ ID NO: 14 - DNA, 23 bp, synthetic construct
  source 1..23: mol_type = other DNA
  modified_base 14: mod_base = OTHER; note = 2'-O-methyladenosine
acagacgaagcggatacctcgtc

SEQ ID NO: 15 - DNA, 24 bp, synthetic construct
  source 1..24: mol_type = other DNA
  modified_base 4: mod_base = OTHER; note = locked nucleic acid base
  modified_base 6: mod_base = OTHER; note = locked nucleic acid base
  modified_base 17: mod_base = OTHER; note = locked nucleic acid base
  modified_base 19: mod_base = OTHER; note = locked nucleic acid base
  modified_base 21: mod_base = OTHER; note = locked nucleic acid base
  modified_base 24: mod_base = OTHER; note = locked nucleic acid base
  modified_base 11: mod_base = cm
  modified_base 15: mod_base = OTHER; note = 2'-O-methyladenosine
acgcagagtcccaaaactattctg

SEQ ID NO: 16 - DNA, 10 bp, synthetic construct
  source 1..10: mol_type = other DNA
  modified_base 9: mod_base = OTHER; note = Phosphorothioated base
acgaagcgga

SEQ ID NO: 17 - DNA, 13 bp, synthetic construct
  source 1..13: mol_type = other DNA
  modified_base 12: mod_base = OTHER; note = Phosphorothioated base
gaaattccaaaga

SEQ ID NO: 18 - DNA, 24 bp, synthetic construct
  source 1..24: mol_type = other DNA
  modified_base 4: mod_base = OTHER; note = locked nucleic acid base
  modified_base 6: mod_base = OTHER; note = locked nucleic acid base
  modified_base 17: mod_base = OTHER; note = locked nucleic acid base
  modified_base 19: mod_base = OTHER; note = locked nucleic acid base
  modified_base 21: mod_base = OTHER; note = locked nucleic acid base
  modified_base 24: mod_base = OTHER; note = locked nucleic acid base
acgcagagtcccaaaactattctg

SEQ ID NO: 19 - DNA, 24 bp, synthetic construct
  source 1..24: mol_type = other DNA
  modified_base 4: mod_base = OTHER; note = locked nucleic acid base
  modified_base 6: mod_base = OTHER; note = locked nucleic acid base
  modified_base 17: mod_base = OTHER; note = locked nucleic acid base
  modified_base 19: mod_base = OTHER; note = locked nucleic acid base
  modified_base 21: mod_base = OTHER; note = locked nucleic acid base
  modified_base 24: mod_base = OTHER; note = locked nucleic acid base
  modified_base 15: mod_base = OTHER; note = 2'-O-methyladenosine
acgcagagtcccaaaactattctg

SEQ ID NO: 20 - DNA, 13 bp, synthetic construct
  source 1..13: mol_type = other DNA
  modified_base 9: mod_base = OTHER; note = Phosphorothioated base
  modified_base 11: mod_base = OTHER; note = Phosphorothioated base
ttcttcatccgaa

SEQ ID NO: 21 - DNA, 12 bp, synthetic construct
  source 1..12: mol_type = other DNA
  modified_base 10: mod_base = OTHER; note = Phosphorothioated base
cagagtcccaaa

SEQ ID NO: 22 - DNA, 11 bp, synthetic construct
  source 1..11: mol_type = other DNA
  modified_base 10: mod_base = OTHER; note = Phosphorothioated base
ccaacaggaga

SEQ ID NO: 23 - DNA, 12 bp, synthetic construct
  source 1..12: mol_type = other DNA
  modified_base 8: mod_base = OTHER; note = Phosphorothioated base
attggattcaag

SEQ ID NO: 24 - DNA, 24 bp, synthetic construct
  source 1..24: mol_type = other DNA
  modified_base 10: mod_base = OTHER; note = locked nucleic acid base
  modified_base 15: mod_base = OTHER; note = locked nucleic acid base
  modified_base 17: mod_base = OTHER; note = locked nucleic acid base
  modified_base 18: mod_base = OTHER; note = locked nucleic acid base
  modified_base 20: mod_base = OTHER; note = locked nucleic acid base
  modified_base 24: mod_base = OTHER; note = locked nucleic acid base
cgccaacaggagaatatgtcctgt

SEQ ID NO: 25 - DNA, 10 bp, synthetic construct
  source 1..10: mol_type = other DNA
  modified_base 8: mod_base = OTHER; note = Phosphorothioated base
ccgttcctaa

SEQ ID NO: 26 - DNA, 11 bp, synthetic construct
  source 1..11: mol_type = other DNA
  modified_base 8: mod_base = OTHER; note = Phosphorothioated base
tgtgctccata

SEQ ID NO: 27 - DNA, 24 bp, synthetic construct
  source 1..24: mol_type = other DNA
  modified_base 6: mod_base = OTHER; note = locked nucleic acid base
  modified_base 15: mod_base = OTHER; note = locked nucleic acid base
  modified_base 16: mod_base = OTHER; note = locked nucleic acid base
  modified_base 17: mod_base = OTHER; note = locked nucleic acid base
  modified_base 18: mod_base = OTHER; note = locked nucleic acid base
  modified_base 24: mod_base = OTHER; note = locked nucleic acid base
acagccgttcctaattcctacggc

SEQ ID NO: 28 - DNA, 10 bp, synthetic construct
  source 1..10: mol_type = other DNA
  modified_base 9: mod_base = OTHER; note = Phosphorothioated base
tctgggagca

SEQ ID NO: 29 - DNA, 11 bp, synthetic construct
  source 1..11: mol_type = other DNA
  modified_base 10: mod_base = OTHER; note = Phosphorothioated base
gctgagactaa

SEQ ID NO: 30 - DNA, 28 bp, synthetic construct
  source 1..28: mol_type = other DNA
  modified_base 6: mod_base = OTHER; note = locked nucleic acid base
  modified_base 17: mod_base = OTHER; note = locked nucleic acid base
  modified_base 18: mod_base = OTHER; note = locked nucleic acid base
  modified_base 21: mod_base = OTHER; note = locked nucleic acid base
  modified_base 24: mod_base = OTHER; note = locked nucleic acid base
  modified_base 28: mod_base = OTHER; note = locked nucleic acid base
gaaccgtctgggagcatgttcttgacgg

SEQ ID NO: 31 - DNA, 13 bp, synthetic construct
  source 1..13: mol_type = other DNA
  modified_base 11: mod_base = OTHER; note = Phosphorothioated base
tcagagtcccaaa

SEQ ID NO: 32 - DNA, 25 bp, synthetic construct
  source 1..25: mol_type = other DNA
  modified_base 6: mod_base = OTHER; note = locked nucleic acid base
  modified_base 7: mod_base = OTHER; note = locked nucleic acid base
  modified_base 17: mod_base = OTHER; note = locked nucleic acid base
  modified_base 18: mod_base = OTHER; note = locked nucleic acid base
  modified_base 20: mod_base = OTHER; note = locked nucleic acid base
  modified_base 25: mod_base = OTHER; note = locked nucleic acid base
  modified_base 16: mod_base = OTHER; note = 2'-O-methyladenosine
acagcagagtcccaaaactatctgc

SEQ ID NO: 33 - DNA, 23 bp, synthetic construct
  source 1..23: mol_type = other DNA
  modified_base 6: mod_base = OTHER; note = locked nucleic acid base
  modified_base 7: mod_base = OTHER; note = locked nucleic acid base
  modified_base 16: mod_base = OTHER; note = locked nucleic acid base
  modified_base 17: mod_base = OTHER; note = locked nucleic acid base
  modified_base 21: mod_base = OTHER; note = locked nucleic acid base
  modified_base 23: mod_base = OTHER; note = locked nucleic acid base
agccagcgaccaaaagaattgct

SEQ ID NO: 34 - DNA, 22 bp, synthetic construct
  source 1..22: mol_type = other DNA
  modified_base 7: mod_base = OTHER; note = locked nucleic acid base
  modified_base 8: mod_base = OTHER; note = locked nucleic acid base
  modified_base 16: mod_base = OTHER; note = locked nucleic acid base
  modified_base 17: mod_base = OTHER; note = locked nucleic acid base
  modified_base 19: mod_base = OTHER; note = locked nucleic acid base
  modified_base 22: mod_base = OTHER; note = locked nucleic acid base
accgcccatccgaattcttggg

SEQ ID NO: 35 - DNA, 22 bp, synthetic construct
  source 1..22: mol_type = other DNA
  modified_base 6: mod_base = OTHER; note = locked nucleic acid base
  modified_base 7: mod_base = OTHER; note = locked nucleic acid base
  modified_base 16: mod_base = OTHER; note = locked nucleic acid base
  modified_base 17: mod_base = OTHER; note = locked nucleic acid base
  modified_base 19: mod_base = OTHER; note = locked nucleic acid base
  modified_base 22: mod_base = OTHER; note = locked nucleic acid base
acgagccatccgaattcttggc

SEQ ID NO: 36 - DNA, 15 bp, synthetic construct
  source 1..15: mol_type = other DNA
  modified_base 13: mod_base = OTHER; note = Phosphorothioated base
gaacggaacaataca

SEQ ID NO: 37 - DNA, 12 bp, synthetic construct
  source 1..12: mol_type = other DNA
  modified_base 10: mod_base = OTHER; note = Phosphorothioated base
cccatattccaa

SEQ ID NO: 38 - DNA, 17 bp, synthetic construct
  source 1..17: mol_type = other DNA
  modified_base 15: mod_base = OTHER; note = Phosphorothioated base
  misc_feature 1..6: note = RNA
gggaacggaacaataca

SEQ ID NO: 39 - DNA, 24 bp, synthetic construct
  source 1..24: mol_type = other DNA
  modified_base 6: mod_base = OTHER; note = locked nucleic acid base
  modified_base 7: mod_base = OTHER; note = locked nucleic acid base
  modified_base 18: mod_base = OTHER; note = locked nucleic acid base
  modified_base 21: mod_base = OTHER; note = locked nucleic acid base
  modified_base 22: mod_base = OTHER; note = locked nucleic acid base
  modified_base 24: mod_base = OTHER; note = locked nucleic acid base
agcacccatattccaatcttcggg

SEQ ID NO: 40 - DNA, 25 bp, synthetic construct
  source 1..25: mol_type = other DNA
  modified_base 6: mod_base = OTHER; note = locked nucleic acid base
  modified_base 7: mod_base = OTHER; note = locked nucleic acid base
  modified_base 18: mod_base = OTHER; note = locked nucleic acid base
  modified_base 20: mod_base = OTHER; note = locked nucleic acid base
  modified_base 22: mod_base = OTHER; note = locked nucleic acid base
  modified_base 25: mod_base = OTHER; note = locked nucleic acid base
cgcacccatattccaatcttcgggt

SEQ ID NO: 41 - DNA, 12 bp, synthetic construct
  source 1..12: mol_type = other DNA
  modified_base 10: mod_base = OTHER; note = Phosphorothioated base
cccatattccaa

SEQ ID NO: 42 - DNA, 24 bp, synthetic construct
  source 1..24: mol_type = other DNA
  modified_base 6: mod_base = OTHER; note = locked nucleic acid base
  modified_base 7: mod_base = OTHER; note = locked nucleic acid base
  modified_base 18: mod_base = OTHER; note = locked nucleic acid base
  modified_base 21: mod_base = OTHER; note = locked nucleic acid base
  modified_base 22: mod_base = OTHER; note = locked nucleic acid base
  modified_base 24: mod_base = OTHER; note = locked nucleic acid base
agcacccatattccaatcttcggg

SEQ ID NO: 43 - DNA, 24 bp, synthetic construct
  source 1..24: mol_type = other DNA
  modified_base 6: mod_base = OTHER; note = locked nucleic acid base
  modified_base 7: mod_base = OTHER; note = locked nucleic acid base
  modified_base 18: mod_base = OTHER; note = locked nucleic acid base
  modified_base 21: mod_base = OTHER; note = locked nucleic acid base
  modified_base 22: mod_base = OTHER; note = locked nucleic acid base
  modified_base 24: mod_base = OTHER; note = locked nucleic acid base
  modified_base 16: mod_base = OTHER; note = 2'-O-methyladenosine
agcacccatattccaatcttcggg

SEQ ID NO: 44 - DNA, 24 bp, synthetic construct
  source 1..24: mol_type = other DNA
  modified_base 6: mod_base = OTHER; note = locked nucleic acid base
  modified_base 7: mod_base = OTHER; note = locked nucleic acid base
  modified_base 18: mod_base = OTHER; note = locked nucleic acid base
  modified_base 21: mod_base = OTHER; note = locked nucleic acid base
  modified_base 22: mod_base = OTHER; note = locked nucleic acid base
  modified_base 24: mod_base = OTHER; note = locked nucleic acid base
  modified_base 14: mod_base = cm
agcacccatattccaatcttcggg

SEQ ID NO: 45 - DNA, 24 bp, synthetic construct
  source 1..24: mol_type = other DNA
  modified_base 6: mod_base = OTHER; note = locked nucleic acid base
  modified_base 7: mod_base = OTHER; note = locked nucleic acid base
  modified_base 18: mod_base = OTHER; note = locked nucleic acid base
  modified_base 21: mod_base = OTHER; note = locked nucleic acid base
  modified_base 22: mod_base = OTHER; note = locked nucleic acid base
  modified_base 24: mod_base = OTHER; note = locked nucleic acid base
  modified_base 14: mod_base = cm
  modified_base 16: mod_base = OTHER; note = 2'-O-methyladenosine
agcacccatattccaatcttcggg